(19) United States
(12) Patent Application Publication
(10) Pub. No.: US 2025/0121003 A1
(43) Pub. Date: Apr. 17, 2025

(54) DEVELOPING INDUCIBLE CLUSTER CHIMERIC ANTIGEN RECEPTOR (CCAR) CONSTRUCTS

(71) Applicant: Dana-Farber Cancer Institute, Inc., Boston, MA (US)

(72) Inventors: Jens G. Lohr, Brookline, MA (US); Birgit Knoechel, Brookline, MA (US)

(21) Appl. No.: 18/704,039

(22) PCT Filed: Nov. 3, 2022
(86) PCT No.: PCT/US2022/079218

§ 371 Date: Apr. 23, 2024

Related U.S. Application Data
(60) Provisional application No. 63/275,752, filed on Nov. 4, 2021.

(51) Int. Cl. A61K 35/17; A61K 40/11; A61K 40/15; A61K 40/31; A61K 40/42; A61K 45/06; A61P 35/00; C07K 16/28; C12N 5/0783; C12N 9/64; C12N 15/86

(52) U.S. Cl. CPC A61K 35/17; A61K 40/11; A61K 40/15; A61K 40/31; A61K 40/4215; A61K 40/4244; A61K 45/06; A61P 35/00; C07K 16/2878; C12N 5/0636; C12N 5/0646; C12N 9/641; C12N 15/86; A61K 2239/13; A61K 2239/17; A61K 2239/21; A61K 2239/22; A61K 2239/28; C12N 2740/15043; C12Y 304/22044

(57) ABSTRACT

Disclosed are cluster CAR and therapeutic payload nucleic acids, immune cells containing them, and uses thereof for controllable adoptive cell therapy and killing CAR T-cell resistant tumor cells.

### RELATED APPLICATIONS

This application claims the benefit of priority under 35 U.S.C. § 119 (e) to U.S. Provisional Application No. 63/275,752, filed Nov. 4, 2021, which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 3, 2022, is named 52095_751001WO_ST.xml and is 157 KB bytes in size.

### BACKGROUND OF THE DISCLOSURE

Almost all types of cancer can develop drug resistance and become refractory to treatment. There are a staggering and diverse number of drug resistance mechanisms, including clonal evolution with competitive outgrowth of genetically distinct cancer cells, epigenetic adaptation of cancer cells with transcriptional changes of cellular states, and alternative splicing events that lead to loss of drug target expression. Overcoming cancer resistance mechanisms and finding cures for cancer patients, particularly with disseminated cancers, remains a major medical need.

Furthermore, tumor cells are known to eliminate immunotherapy targets on their cells via several cell-intrinsic mechanisms, including down-regulation of the target protein on the cancer cell surface through transcriptional state changes and epigenetic adaptation, genetic deletion of the target protein, mutation of the target protein, removal of the immunotherapeutic binding site through splicing, target masking through conformational changes and production related mechanisms.

Therefore, there is a need to deliver effective therapeutics specifically to tumor sites while sparing normal, non-diseased tissue. Such a method and system would enable the effective administration of therapeutics that would be otherwise highly toxic if administered systematically. They would also be particularly attractive because most neoplasms tend to grow and metastasize in clusters rather than being homogenously distributed across the body.

### SUMMARY OF THE DISCLOSURE

A first aspect of the present disclosure is directed to a nucleic acid construct that contains at least one of three nucleic acids. The first nucleic acid contains a first promoter operably linked to a nucleic acid encoding a first chimeric antigen receptor (also referred to herein as the “protease CAR”) containing a first extracellular domain which has a first antigen binding domain that binds a first tumor associated antigen (TAA), a first transmembrane domain, and a first intracellular domain that includes a first signaling domain, and a protease domain. The second nucleic acid of the three nucleic acids contains a second promotor operably linked to a nucleic acid encoding a second CAR (also referred to herein as the “activation CAR”) containing a second extracellular domain comprising a second antigen binding domain that binds a TAA, a second transmembrane domain, and a second intracellular domain that contains a second signaling domain, a cleavage site recognized by the protease, and a transcriptional activator. The third nucleic acid of the three nucleic acids contains a transcriptional acceptor that binds the transcriptional activator, a third promoter and a nucleic acid encoding a leader peptide and a therapeutic payload that is operatively linked to the third promoter.

Another aspect of the present disclosure is directed to a vector containing the nucleic acid construct encoding the Protease CAR, the Activation CAR, the third nucleic acid, subcombinations of two of the three nucleic acids, or all three nucleic acids.

Yet another aspect of the present disclosure is directed to a method of producing a genetically modified immune cell. The method entails introducing a first nucleic acid construct, a second nucleic acid construct, and a third nucleic acid construct into an immune cell, wherein: the first nucleic acid construct contains a promoter operably linked to a nucleic acid encoding a Protease CAR containing an extracellular domain containing an antigen biding domain that binds a first TAA, a transmembrane domain, and an intracellular domain containing a first signaling domain a protease domain; the second nucleic acid construct contains a promoter operably linked to a nucleic acid encoding an Activation CAR containing an extracellular domain containing an antigen biding domain that binds a second TAA, a transmembrane domain, and an intracellular domain containing a second signaling domain, a cleavage site recognized by the protease, and a transcriptional activator; and the third nucleic acid construct contains a transcriptional acceptor that binds the transcriptional activator, a third promoter, and a nucleic acid encoding a leader peptide and a therapeutic payload that is operatively linked to the third nucleic acid construct's promoter.

Yet another aspect of the present disclosure is directed to a genetically modified immune cell containing the first nucleic acid, the second nucleic acid, and the third nucleic acid.

Yet another aspect of the present disclosure is directed to a pharmaceutical composition containing a therapeutically effective number of the genetically modified immune cells and a pharmaceutically acceptable carrier.

Another aspect of the present disclosure is directed to a method of treating cancer. The method entails administering to a subject in need thereof, the pharmaceutical composition.

Not intending to be bound by theory of operation, Applicant believes that upon administration to a cancer patient, the genetically modified immune cells achieve therapeutic efficacy via a “cluster effect” in that upon binding to cancer cells, the immune cells, referred to herein as cluster CAR (cCAR) cells, produce a therapeutic payload for expression on the membrane of the immune cell, or release into the extracellular space which then binds and exerts a therapeutic effect (e.g., killing) on other cancer cells in the immediate proximity. Working examples disclosed herein demonstrate how a therapeutic cluster CAR system, applicable to immune cells (e.g., T cells, NK cells) allow for the delivery of therapeutics efficiently to tumor sites while substantially sparing healthy tissue.

### BRIEF DESCRIPTION OF THE DRAWINGS

FIG. 1 schematically illustrates an aspect of the cCAR system in which cells deliver a therapeutic payload in the vicinity of a tumor cell. The cCAR system combines CAR-mediated killing with CAR-independent killing through the delivery of a therapeutic payload. cCAR cells express a Protease CAR and an Activation CAR which engage either the same or two different surface TAAs. CAR engagement results in CAR-specific killing (inner box) as well as therapeutic payload-dependent killing (inner circle). The therapeutic payload will only be present at effective concentrations in the vicinity of the cCAR cell and will therefore spare non-diseased cells present in a safe zone (outer circle) outside of the inner circle.

FIG. 2 schematically illustrates domains of an anti-BCMA Protease CAR, an anti-BCMA Activation CAR, and a third nucleic acid encoding a therapeutic payload.

FIGS. 3A-3H are a series of schematics showing how cCAR cells are produced and act to provide tumor-specific delivery of therapeutic payloads. Recognition of two tumor surface proteins by the Protease CAR and the Activation CAR on T cells leads to coalescence of the CARs at the immunological synapse, which triggers a cascade of events that leads to transcription and secretion of the therapeutic payload, in addition to CAR-mediated cytotoxicity.

FIGS. 4A-4D are a series of flow cytometry plots showing an embodiment of cCAR cells expressing two or more of the Protease CAR, the Activation CAR, the third nucleic acid encoding a therapeutic payload, where the Protease CAR additionally encodes a myc-tag reporter; the Activation CAR encodes a truncated EGFR reporter; and the third nucleic acid additionally encodes a mCherry reporter. The Protease CAR is detected with an anti-myc-APC antibody; the Activation CAR is detected with an anti-EGFR-PE antibody and third nucleic acid is detected directly by the mCherry peptide. FIG. 4A is a series of flow cytometry plots of cells expressing the Protease CAR, the Activation CAR, and third nucleic acid. FIG. 4B is a series of flow cytometry plots of cells expressing the Activation CAR and the third nucleic acid. FIG. 4C is a series of flow cytometry plots of cells expressing the Protease CAR and third nucleic acid. FIG. 4D is a series of flow cytometry plots of cells expressing the Protease CAR and the third nucleic acid.

FIG. 4E is a schematic illustration of embodiments of the Protease CAR, the Activation CAR, and the third nucleic acid that were used to generate the flow cytometry plots of FIGS. 4A-4D.

FIGS. 5A-5C are schematics illustrating vectors that contain nucleic acids encoding the Protease CAR and the Activation CAR, and the third nucleic acid.

FIG. 6 is a bar plot that shows cCAR cells kill target cancer cells when expressing both of the Protease CAR and the Activation CAR, as well as the third nucleic acid, determined as a function of the ratio of live OPM2 cells to control beads.

FIG. 7 is a bar plot that shows cCAR cells express GFP as a model therapeutic payload when all of the Protease CAR, the Activation CAR, and the third nucleic acid are expressed, as determined by the percentage of GFP⁺ cCAR cells.

FIGS. 8A-8C are a set of flow cytometry plots and a bar pot that shows cCAR cells target killing. FIG. 8A is a flow cytometry plot showing BCMA surface expression on OPM2 cells. FIG. 8B is a flow cytometry plot showing BCMA surface expression on NALM-6 cells.

FIG. 8C is a bar plot showing killing of target NALM-6 cells after co-culture with control T cells or cCAR T cells, determined as a function of the ratio of live NALM-6 cells to control beads.

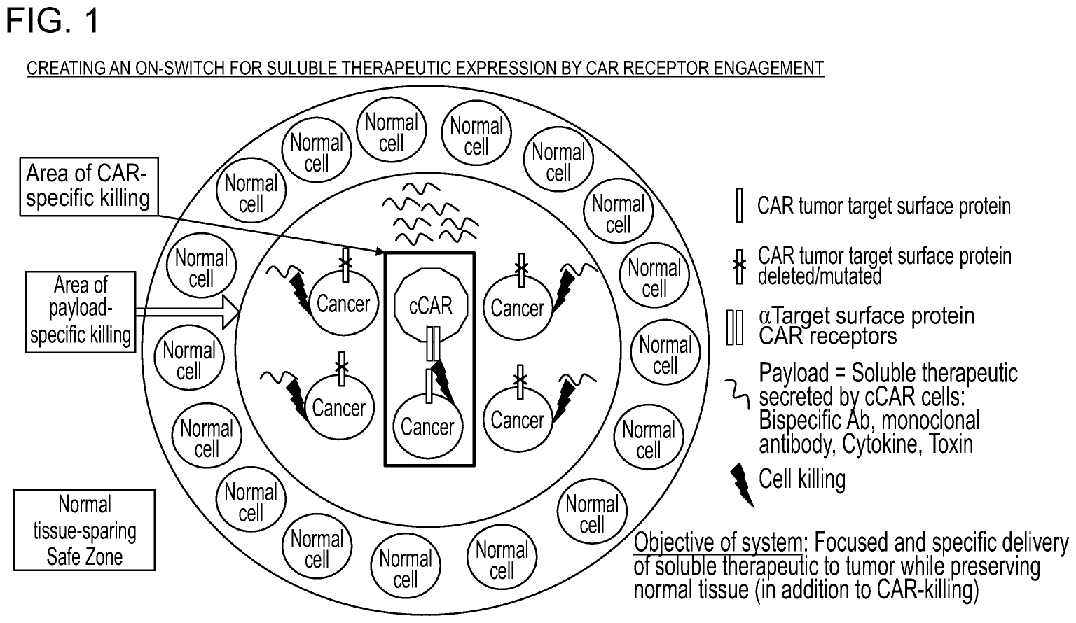

### DETAILED DESCRIPTION OF THE DISCLOSURE

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” mean “one or more” and therefore include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The transitional term “comprising,” which is synonymous with “include(s)”, “including,” “contain(s)”, “containing,” or “characterized by,” is inclusive or open-ended and does not exclude additional, unrecited elements or method steps. By contrast, the transitional phrases “consist(s) of” and “consisting of” excludes any element or method step not specified in the claim (or the specific element or method step with which the phrase “consisting of” is associated). The transitional phrase “consisting essentially of” limits the scope of a claim to the specified elements and method or steps and “unrecited elements and method steps that do not materially affect the basic and novel characteristic(s)” of the claimed disclosure.

### Nucleic Acid Constructs

In one aspect, the disclosure provides a nucleic acid construct that contains at least one of three nucleic acids, wherein the first nucleic acid contains a promoter operably linked to a nucleic acid encoding a first Protease chimeric antigen receptor (CAR) including an extracellular domain which has a first antigen binding domain that binds a first TAA, a transmembrane domain, and an intracellular domain that contains a protease domain. The second nucleic acid of the three nucleic acids contains a promotor operably linked to a nucleic acid encoding an Activation CAR including an extracellular domain comprising an antigen binding domain that binds a second TAA, a transmembrane domain, and an intracellular domain that contains a cleavage site recognized by the protease and a transcriptional activator. The third nucleic acid of the three nucleic acids contains a transcriptional acceptor that binds the transcriptional activator, a promoter and a nucleic acid encoding a leader peptide and a therapeutic payload that is operatively linked to the promoter of the third nucleic acid.

The terms “antigen” and “TAA” as used herein refers to a target molecule expressed by a cancer cell. Antigens may be proteins, peptides, peptide-protein complexes (e.g., a peptide bound to an MHC molecule), protein-carbohydrate complexes (e.g., a glycoprotein), protein-lipid complexes (e.g., a lipoprotein), protein-nucleic acid complexes (e.g., a nucleoprotein), carbohydrates, lipids, or nucleic acids.

As known in the art, the term “nucleic acid” as used herein refers to a polymer of nucleotides, each of which are organic molecules consisting of a nucleoside (a nucleobase and a five-carbon sugar) and a phosphate. The term nucleotide, unless specifically sated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA). Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). Nucleic acids are linear chains of nucleotides (e.g., at least 3 nucleotides) chemically bonded by a series of ester linkages between the phosphoryl group of one nucleotide and the hydroxyl group of the sugar (i.e., ribose or 2′-deoxyribose) in the adjacent nucleotide. In the present context, it is understood that the nucleic acids are exogenous to the immune cells into which they may be introduced.

The term “promoter” as used herein refers to a non-coding nucleic acid that regulates, directly or indirectly, the transcription of a corresponding nucleic acid coding sequence to which it is operably linked, which in the context of the present disclosure is a CAR or a therapeutic payload. A promoter may function alone to regulate transcription, or it may act in concert with one or more other regulatory sequences (e.g., enhancers or silencers, or regulatory elements that may be present in the expression vector). Promoters are located near the transcription start sites of genes, on the same strand and upstream on the DNA (towards the 5′ region of the sense strand). Promoters typically range from about 100-1000 base pairs in length.

The term “operatively linked” as used herein is to be understood that the nucleic acid coding sequence is spatially situated or disposed in the nucleic acid construct relative to a promoter to drive the expression of the protein encoded by the nucleic acid coding sequence.

In some embodiments, the nucleic acid construct includes two of the first, the second, and the third nucleic acids. In some embodiments, the nucleic acid construct includes the first, the second, and the third nucleic acids.

The expression of the nucleic acids encoding the Protease CAR, the Activation CAR, and the therapeutic payload is each controlled by a promoter, which may be a native promoter or a synthetic promoter. In some embodiments, one or more of the promoters are derived from the elongation factor 1 Alpha (EF-1α), cytomegalovirus (CMV), β-actin, a simian virus 40 (SV40) early promoter, human phosphoglycerate kinase (PGK), RPBSA (synthetic, from Sleeping Beauty), or CAG (synthetic, CMV early enhancer element, chicken β-Actin, and splice acceptor of rabbit β-Globin) promoter. The term “derived from” as used herein when referring to protein or nucleic acid sequences refers to a sequence that originates from another, parent sequence. A sequence derived from a parent sequence may be identical, may be a portion of the parent sequence, or may have at least one variant from the parent sequence. Variants may include substitutions, insertions, or deletions. Thus, for example, an amino acid sequence derived from a parent sequence may be identical for a specific range of amino acids of the parent but does not include amino acids outside that specific region.

In some embodiments, the promoter may have a core region located close to the beginning of the nucleic acid coding sequence. In some embodiments, the promoter is modified relative to a native promoter. One modification entails the removal of methylation sensitive sites (e.g., a cytosine nucleotide is followed by a guanine nucleotide, or “CpG”). Another modification entails the addition of a regulatory sequence that binds DNA methylation repressive transcriptional factors. In some embodiments, the expression vector includes A/T-rich, nuclear matrix interacting sequences, known as scaffold matrix attachment regions (S/MAR), which may enhance transformation efficiency and improve the stability of transgene expression.

The first, the second, and the third promoters may be the same or different. In some embodiments, the first, the second, and the third promoters are different. In some embodiments, the first and the second promoters are the same and the third promoter is different from the first and the second promoters. In some embodiments, the first and the third promoters are the same and the second promoter is different. In some embodiments, the second and the third promoters are the same and the first promoter is different.

The EF-1a promoter is provided at NCBI Accession No. J04617.1. Variations of modified CMV promoters are provided at NCBI Accession Nos. AY218848, AF477200, M64754, and AF286076. The PGK promoter is provided at NCBI Accession No. NC_000023.11, range 78104248 to 78129295. The RPBSA promoter is provided in NCBI Accession No. MN811119.1. The CAG promoter is provided in NCBI Accession No. MG763233.1. In some embodiments, one or more of the promoters are derived from EF-1α. In some embodiments, the first and the second promoters are derived from EF-1α. In some embodiments, the first and the second promoters are derived from EF-1a and the third promoter is derived from CMV.

The antigen binding domain of the Protease CAR (also referred to herein as the “first antigen binding domain”) and the antigen binding domain of the Activation CAR (also referred to herein as the “second antigen binding domain”) each bind a TAA. The TAAs may be the same or different. In some embodiments, either or both the first and second antigen binding domains bind BCMA, CD19, CD20, CD38, CD138, FCRH5, GPRC5D, or SLAMF7.

In some embodiments, the first and/or the second antigen binding domain is an antibody fragment. In some embodiments, the first and/or the second antigen binding domain is a single-chain variable antibody fragment (scFv) containing a variable heavy (VH) and a variable light (VL) domain. In some embodiments, the first and/or the second antigen binding domains contain the variable domain of an antibody light chain and the variable domain of an antibody heavy chain interconnected by a linker.

In some embodiments, the first and/or the second antigen binding domain binds BCMA. In some embodiments, the antigen binding domain is derived from a commercially available anti-BCMA antibody, and BCMA-binding fragments thereof, or derivative thereof, e.g., belantamab (Blenrep®), linvoseltamab (REGN5458), pacanalotamab (AMG 420), pavurutamab (AMG 701) and teclistamab (Tecvayli®), the amino acid sequences of the heavy and light chains of which are set forth in Table 1. In some embodiments, the first and/or the second antigen binding domains contain the variable domain of the light chain and the variable domain of the heavy chain of an anti-BCMA antibody, the variable domains connected by a linker.

TABLE 1
Amino Acid Sequences of anti-BCMA antibody fragments

| Polypeptide | Sequence | |
|---|---|---|
| belantamab | 1 | qvqlvqsgae vkkpgssvkv sckasggtfs nywmhwvrqa pgqglewmga tyrghsdtyy |
| heavy chain | 61 | nqkfkgrvti tadkststay melsslrsed tavyycarga iydgydvldn wgqgtlvtvs |
| (SEQ ID NO: | 121 | sastkgpsvf plapssksts ggtaalgclv kdyfpepvtv swnsgaltsg vhtfpavlqs |
| 1) | 181 | sglyslssvv tvpssslgtq tyicnvnhkp sntkvdkkve pkscdkthtc ppcpapellg |
| | 241 | gpsvflfppk pkdtlmisrt pevtcvvvdv shedpevkfn wyvdgvevhn aktkpreeqy |
| | 301 | nstyrvvsvl tvlhqdwlng keykckvsnk alpapiekti skakgqprep qvytlppsrd |
| | 361 | eltknqvslt clvkgfypsd iavewesngq pennykttpp vldsdgsffl yskltvdksr |
| | 421 | wqqgnvfscs vmhealhnhy tqkslslspg k |
| belantamab | 1 | diqmtqspss lsasvgdrvt itcsasqdis nylnwyqqkp gkapklliyy tsnlhsgvps |
| light chain | 61 | rfsgsgsgtd ftltisslqp edfatyycqq yrklpwtfgq gtkleikrtv aapsvfifpp |
| (SEQ ID NO: | 121 | sdeqlksgta svvcllnnfy preakvqwkv dnalqsgnsq esvteqdskd styslsstlt |
| 2) | 181 | 1skadyekhk vyacevthqg lsspvtksfn rgec |
| linvoseltamab | 1 | evqlvesggg lvqpgrslrl scaasgftfd dysmhwvrqa pgkglewvsg iswnsgskgy |
| heavy chain | 61 | adsvkgrfti srdnaknsly lqmnslraed talyycakyg sgygkfyhyg ldvwgqgttv |
| (SEQ ID NO: | 121 | tvssastkgp svfplapcsr stsestaalg clvkdyfpep vtvswnsgal tsgvhtfpav |
| 3) | 181 | lqssglysls svvtvpsssl gtktytcnvd hkpsntkvdk rveskygppc ppcpappvag |
| | 241 | psvflfppkp kdtlmisrtp evtcvvvdvs qedpevqfnw yvdgvevhna ktkpreeqfn |
| | 301 | styrvvsvlt vlhqdwlngk eykckvsnkg lpssiektis kakgqprepq vytlppsqee |
| | 361 | mtknqvsltc lvkgfypsdi avewesngqp ennykttppv ldsdgsffly srltvdksrw |
| | 421 | qegnvfscsv mhealhnrft qkslslspgk |
| linvoseltamab | 1 | diqmtqspss lsasvgdrvt itcrasqsis sylnwyqqkp gkapklliya asslqsgvps |
| light chain | 61 | rfsgsgsgtd ftltisslqp edfatyycqq systppitfg qgtrleikrt vaapsvfifp |
| (SEQ ID NO: | 121 | psdeqlksgt asvvcllnnf ypreakvqwk vdnalqsgns qesvteqdsk dstyslsstl |
| 4) | 181 | tlskadyekh kvyacevthq glsspvtksf nrgec |
| pacanalotamab | 1 | qvqlvqsgae vkkpgasvkv sckasgytft nhiihwvrqa pgqglewmgy inpypgyhay |
| (SEQ ID NO: | 61 | nekfqgratm tsdtststvy melsslrsed tavyycardg yyrdtdvldy wgqgtlvtvs |
| 5) | 121 | sggggsgggg sggggsdiqm tqspsslsas vgdrvtitcq asqdisnyln wyqqkpgkap |
| | 181 | klliyytsrl htgvpsrfsg sgsgtdftft isslepedia tyycqqgntl pwtfgqgtkv |
| | 241 | eiksggggse vqlvesgggl vqpggslkls caasgftfnk yamnwvrqap gkglewvari |
| | 301 | rskynnyaty yadsvkdrft isrddsknta ylqmnnlkte dtavyycvrh gnfgnsyisy |
| | 361 | waywgqgtlv tvssggggsg gggsggggsq tvvtqepslt vspggtvtlt cgsstgavts |
| | 421 | gnypnwvqqk pgqaprglig gtkflapgtp arfsgsllgg kaaltlsgvq pedeaeyycv |
| | 481 | lwysnrwvfg ggtkltvlhh hhhh |
| pavurutamab | 1 | vspggtvtlt cgsstgavts gnypnwvqqk pgqaprglig gtkflapgtp arfsgsllgg |
| heavy chain | 61 | kaaltlsgvq pedeaeyycv lwysnrwvfg ggtkltvlgg ggdkthtcpp cpapellggp |
| (SEQ ID NO: | 121 | svflfppkpk dtlmisrtpe vtcvvvdvsh edpevkfnwy vdgvevhnak tkpceeqygs |
| 6): | 181 | tyrcvsvltv lhqdwlngke ykckvsnkal papiektisk akgqprepqv ytlppsreem |
| | 241 | tknqvsltcl vkgfypsdia vewesngqpe nnykttppvl dsdgsfflys kltvdksrwq |
| | 301 | qgnvfscsvm healhnhytq kslslspgkg gggsggggsg gggsggggsg gggsggggsd |
| | 361 | kthtcppcpa pellggpsvf lfppkpkdtl misrtpevtc vvvdvshedp evkfnwyvdg |
| | 421 | vevhnaktkp ceeqygstyr cvsvltvlhq dwlngkeykc kvsnkalpap iektiskakg |
| | 481 | qprepqvytl ppsreemtkn qvsltclvkg fypsdiavew esngqpenny kttppvldsd |
| | 541 | gsfflysklt vdksrwqqgn vfscsvmhea lhnhytqksl slspgk |
| pavurutamab | 1 | qvqlvqsgae vkkpgasvkv sckasgytft nhiihwvrqa pgqclewmgy inpypgyhay |
| light chain | 61 | nekfqgratm tsdtststvy melsslrsed tavyycardg yyrdtdvldy wgqgtlvtvs |
| (SEQ ID NO: | 121 | sggggsgggg sggggsdiqm tqspsslsas vgdrvtitcq asqdisnyln wyqqkpgkap |
| 7) | 181 | klliyytsrl htgvpsrfsg sgsgtdftft isslepedia tyycqqgntl pwtfgcgtkv |
| | 241 | eiksggggse valvesgggl vqpggslkls caasgftfnk yamnwvrqap gkglewvari |
| | 301 | rskynnyaty yadsvkdrft isrddsknta ylqmnnlkte dtavyycvrh gnfgnsyisy |
| | 361 | waywgqgtlv tvssggggsg gggsggggsq tvvtqepslt |
| teclistamab | 1 | qlqlqesgpg lvkpsetlsl tctvsggsis sgsyfwgwir qppgkglewi gsiyysgity |
| anti-BCMA | 61 | ynpslksrvt isvdtsknqf slklssvtaa dtavyycarh dgavaglfdy wgqgtlvtvs |
| heavy chain | 121 | sastkgpsvf plapcsrsts estaalgclv kdyfpepvtv swnsgaltsg vhtfpavlqs |
| (SEQ ID NO: | 181 | sglyslssvv tvpssslgtk tytcnvdhkp sntkvdkrve skygppcppc papeaaggps |
| 8) | 241 | vflfppkpkd tlmisrtpev tcvvvdvsqe dpevqfnwyv dgvevhnakt kpreeqfnst |
| | 301 | yrvvsvltvl hqdwlngkey kckvsnkglp ssiektiska kgqprepqvy tlppsqeemt |
| | 361 | knqvsltclv kgfypsdiav ewesngqpen nykttppvld sdgsfflysr ltvdksrwqe |
| | 421 | gnvfscsvmh ealhnhytqk slslslgk |
| teclistamab | 1 | syvltqppsv svapgqtari tcggnnigsk svhwyqqppg qapvvvvydd sdrpsgiper |
| anti-BCMA | 61 | fsgsnsgnta tltisrveag deavyycqvw dsssdhvvfg ggtkltvlgq pkaapsvtlf |
| light chain | 121 | ppsseelqan katlvclisd fypgavtvaw kgdsspvkag vetttpskqs nnkyaassyl |
| (SEQ ID NO: | 181 | sltpeqwksh rsyscqvthe gstvektvap tecs |
| 9) | | |

In some embodiments, the first and/or the second antigen binding domain contains the VL having the amino acid sequence set forth below (SEQ ID NO: 10):

| | |
|---|---|
| 1 | diqmtqspss lsasvgdrvt itcsasqdis nylnwyqqkp gkapklliyy tsnlhsgvps |
| 61 | rfsgsgsgtd ftltisslqp edfatyycqq yrklpwtfgq gtkleik |

Additionally, the first and/or the second antigen binding domain contains the VH having the amino acid sequence set forth below (SEQ ID NO: 11):

| | |
|---|---|
| 1 | qvqlvqsgae vkkpgssvkv sckasggtfs nywmhwvrqa pgqglewmga tyrghsdtyy |
| 61 | nqkfkgrvti tadkststay melsslrsed tavyycarga iydgydvldn wgqgtlvtvs |
| 121 | s |

Additional anti-BCMA binding domains are known in the art. See, e.g., U.S. Pat. Nos. 10,072,088 and 11,084,880 and U.S. Patent Application Publications 2016/0131655, 2017/0226216, 2018/0133296, 2019/0151365, 2019/0381171, 2020/0339699, 2020/0055948, and 2022/0064316.

In some embodiments, the first and/or the second antigen binding domain binds CD19. In some embodiments, the antigen binding domain is derived from a commercially available anti-CD19 antibody, anti-CD19-binding fragments thereof, or derivative thereof, e.g., loncastuximab (Zynlonta®), tafasitamab (Monjuvi®), denintuzumab (SGN-CD19A), and inebilizumab (Uplizna®), the amino acid sequences of the heavy and light chains of which are set forth in Table 2:

TABLE 2
Amino Acid Sequences of anti-CD19 antibody fragments

| Polypeptide | Sequence | |
|---|---|---|
| loncastuximab | 1 | qvqlvqpgae vvkpgasvkl scktsgytft snwmhwvkqa pgqglewige idpsdsytny |
| heavy chain | 61 | nqnfqgkakl tvdkststay mevsslrsdd tavyycargs npyyyamdyw gqgtsvtvss |
| (SEQ ID NO: | 121 | astkgpsvfp lapsskstsg gtaalgclvk dyfpepvtvs wnsgaltsgv htfpavlqss |
| 12) | 181 | glyslssvvt vpssslgtqt yicnvnhkps ntkvdkkvep kscdkthtcp pcpapellgg |
| | 241 | psvflfppkp kdtlmisrtp evtcvvvdvs hedpevkfnw yvdgvevhna ktkpreeqyn |
| | 301 | styrvvsvlt vlhqdwlngk eykckvsnka lpapiektis kakgqprepq vytlppsree |
| | 361 | mtknqvsltc lvkgfypsdi avewesngqp ennykttppv ldsdgsffly skltvdksrw |
| | 421 | qqgnvfscsv mhealhnhyt qkslslspg |
| loncastuximab | 1 | eivltqspai msaspgervt mtcsassgvn ymhwyqqkpg tsprrwiydt sklasgvpar |
| light chain | 61 | fsgsgsgtsy sltissmepe daatyychqr gsytfgggtk leikrtvaap svfifppsde |
| (SEQ ID NO: | 121 | qlksgtasvv cllnnfypre akvqwkvdna lqsgnsqesv teqdskdsty slsstltlsk |
| 13) | 181 | adyekhkvya cevthqglss pvtksfnrge c |
| tafasitamab | 1 | evqlvesggg lvkpggslkl scaasgytft syvmhwvrqa pgkglewigy inpyndgtky |
| heavy chain | 61 | nekfqgrvti ssdksistay melsslrsed tamyycargt yyygtrvfdy wgqgtlvtvs |
| (SEQ ID NO: | 121 | sastkgpsvf plapssksts ggtaalgclv kdyfpepvtv swnsgaltsg vhtfpavlqs |
| 14) | 181 | sglyslssvv tvpssslgtq tyicnvnhkp sntkvdkkve pkscdkthtc ppcpapellg |
| | 241 | gpdvflfppk pkdtlmisrt pevtcvvvdv shedpevqfn wyvdgvevhn aktkpreeqf |
| | 301 | nstfrvvsvl tvvhqdwlng keykckvsnk alpapeekti sktkgqprep qvytlppsre |
| | 361 | emtknqvslt clvkgfypsd iavewesngq pennykttpp mldsdgsffl yskltvdksr |
| | 421 | wqqgnvfscs vmhealhnhy tqkslslspg k |
| tafasitamab | 1 | divmtqspat lslspgerat lscrsskslq nvngntylyw fqqkpgqspq lliyrmsnln |
| light chain | 61 | sgvpdrfsgs gsgteftlti sslepedfav yycmqhleyp itfgagtkle ikrtvaapsv |
| (SEQ ID NO: | 121 | fifppsdeql ksgtasvvcl lnnfypreak vqwkvdnalq sgnsqesvte qdskdstysl |
| 15) | 181 | sstltlskad yekhkvyace vthqglsspv tksfnrgec |
| denintuzumab | 1 | xvqlqesgpg lvkpsqtlsl tctvsggsis tsgmgvgwir qhpgkglewi ghiwwdddkr |
| heavy chain | 61 | ynpalksrvt isvdtsknqf slklssvtaa dtavyycarm elwsyyfdyw gqgtlvtvss |
| (SEQ ID NO: | 121 | astkgpsvfp lapsskstsg gtaalgclvk dyfpepvtvs wnsgaltsgv htfpavlqss |
| 16) | 181 | glyslssvvt vpssslgtqt yicnvnhkps ntkvdkkvep kscdkthtcp pcpapellgg |
| | 241 | psvflfppkp kdtlmisrtp evtcvvvdvs hedpevkfnw yvdgvevhna ktkpreeqyn |
| | 301 | styrvvsvlt vlhqdwlngk eykckvsnka lpapiektis kakgqprepq vytlppsrde |
| | 361 | ltknqvsltc lvkgfypsdi avewesngqp ennykttppv ldsdgsffly skltvdksrw |
| | 421 | qqgnvfscsv mhealhnhyt qkslslspg |
| denintuzumab | 1 | eivltqspat lslspgerat lscsasssvs ymhwyqqkpg qaprlliydt sklasgipar |
| light chain (SEQ | 61 | fsgsgsgtdf tltisslepe dvavyycfqg svypftfgqg tkleikrtva apsvfifpps |
| ID NO: 17) | 121 | deqlksgtas vvcllnnfyp reakvqwkvd nalqsgnsqe svteqdskds tyslsstltl |
| | 181 | skadyekhkv yacevthqgl sspvtksfnr gec |
| inebilizumab | 1 | evqlvesggg lvqpggslrl scaasgftfs sswmnwvrqa pgkglewvgr iypgdgdtny |
| heavy chain | 61 | nvkfkgrfti srddsknsly lqmnslkted tavyycarsg fittvrdfdy wgqgtlvtvs |
| (SEQ ID NO: | 121 | sastkgpsvf plapssksts ggtaalgclv kdyfpepvtv swnsgaltsg vhtfpavlqs |
| 18) | 181 | sglyslssvv tvpssslgtq tyicnvnhkp sntkvdkrve pkscdkthtc ppcpapellg |
| | 241 | gpsvflfppk pkdtlmisrt pevtcvvvdv shedpevkfn wyvdgvevhn aktkpreegy |
| | 301 | nstyrvvsvl tvlhqdwlng keykckvsnk alpapiekti skakgqprep qvytlppsre |
| | 361 | emtknqvslt clvkgfypsd iavewesngq pennykttpp vldsdgsffl yskltvdksr |
| | 421 | wqqgnvfscs vmhealhnhy tqkslslspg k |
| inebilizumab | 1 | eivltqspdf qsvtpkekvt itcrasesvd tfgisfmnwf qqkpdqspkl liheasnqgs |
| light chain | 61 | gvpsrfsgsg sgtdftltin sleaedaaty ycqqskevpf tfgggtkvei krtvaapsvf |
| (SEQ ID NO: | 121 | ifppsdeqlk sgtasvvcll nnfypreakv qwkvdnalqs gnsqesvteq dskdstysls |
| 19) | 181 | stltlskady ekhkvyacev thqglsspvt ksfnrgec |
| obexelimab | 1 | evqlvesggg lvkpggslkl scaasgytft syvmhwvrqa pgkglewigy inpyndgtky |
| heavy chain | 61 | nekfqgrvti ssdksistay melsslrsed tamyycargt yyygtrvfdy wgqgtlvtvs |
| (SEQ ID NO: | 121 | sastkgpsvf plapssksts ggtaalgclv kdyfpepvtv swnsgaltsg vhtfpavlqs |
| 20) | 181 | sglyslssvv tvpssslgtq tyicnvnhkp sntkvdkkve pkscdkthtc ppcpapellg |
| | 241 | gpsvflfppk pkdtlmisrt pevtcvvvdv ehedpevkfn wyvdgvevhn aktkpreeqy |
| | 301 | nstyrvvsvl tvlhqdwlng keykckvsnk afpapiekti skakgqprep qvytlppsre |
| | 361 | emtknqvslt clvkgfypsd iavewesngq pennykttpp vldsdgsffl yskltvdksr |
| | 421 | wqqgnvfscs vmhealhnhy tqkslslspg k |
| obexelimab | 1 | divmtqspat lslspgerat lscrsskslq nvngntylyw fqqkpgqspq lliyrmsnln |
| light chain | 61 | sgvpdrfsgs gsgteftlti sslepedfav yycmqhleyp itfgagtkle ikrtvaapsv |
| (SEQ ID NO: | 121 | fifppsdeql ksgtasvvcl lnnfypreak vqwkvdnalq sgnsqesvte qdskdstysl |
| 21) | 181 | sstltlskad yekhkvyace vthqglsspv tksfnrgec |

In some embodiments, the first and/or the second antigen binding domain, e.g., a scFv, binds CD20. In some embodiments, the antigen binding domain is derived from a commercially available anti-CD20 antibody, CD20-binding fragments thereof, or derivative thereof, e.g., ofatumumab (Arzerra®, Kesimpta®), veltuzumab (IMMU-106), tositumomab (Bexxar®), and rituximab (Rituxan®, Riabni®, Truximab®), the amino acid sequences of the heavy and light chains of which are set forth in Table 3:

TABLE 3
Amino Acid Sequences of anti-CD20 antibody fragments

| Polypeptide | Sequence | |
|---|---|---|
| ofatumumab | 1 | evqlvesggg lvqpgrslrl scaasgftfn dyamhwvrqa pgkglewvst iswnsgsigy |
| heavy chain | 61 | adsvkgrfti srdnakksly lqmnslraed talyycakdi qygnyyygmd vwgqgttvtv |
| (SEQ ID NO: | 121 | ssastkgpsv fplapgssks tsgtaalgcl vkdyfpepvt vswnsgalts gvhtfpavlq |
| 22) | 181 | ssglyslssv vtvpssslgt qtyicnvnhk psntkvdkkv ep |
| ofatumumab | 1 | eivltqspat lslspgerat lscrasqsvs sylawyqqkp gqaprlliyd asnratgipa |
| light chain | 61 | rfsgsgsgtd ftltisslep edfavyycqq rsnwpitfgq gtrleikrtv aapsvfifpp |
| (SEQ ID NO: | 121 | sdeqlksgta svvcllnnfy preakvqwkv dnalqsgnsq esvteqdskd styslsstlt |
| 23) | 181 | lskadyekhk vyacevthqg lsspvtksfn r |
| veltuzumab | 1 | qvqlqqsgae vkkpgssvkv sckasgytft synmhwvkqa pgqglewiga iypgngdtsy |
| heavy chain | 61 | nqkfkgkatl tadestntay melsslrsed tafyycarst yyggdwyfdv wgqgttvtvs |
| (SEQ ID NO: | 121 | sastkgpsvf plapssksts ggtaalgclv kdyfpepvtv swnsgaltsg vhtfpavlqs |
| 24) | 181 | sglyslssvv tvpssslgtq tyicnvnhkp sntkvdkrve pkscdkthtc ppcpapellg |
| | 241 | gpsvflfppk pkdtlmisrt pevtcvvvdv shedpevkfn wyvdgvevhn aktkpreegy |
| | 301 | nstyrvvsvl tvlhqdwlng keykckvsnk alpapiekti skakgqprep qvytlppsre |
| | 361 | emtknqvslt clvkgfypsd iavewesngq pennykttpp vldsdgsffl yskltvdksr |
| | 421 | wqqgnvfscs vmhealhnhy tqkslslspg k |
| veltuzumab | 1 | diqltqspss lsasvgdrvt mtcrasssvs yihwfqqkpg kapkpwiyat snlasgvpvr |
| light chain | 61 | fsgsgsgtdy tftisslqpe diatyycqqw tsnpptfggg tkleikrtva apsvfifpps |
| (SEQ ID NO: | 121 | deqlksgtas vvcllnnfyp reakvqwkvd nalqsgnsqe svteqdskds tyslsstltl |
| 25) | 181 | skadyekhkv yacevthqgl sspvtksfnr gec |
| tositumomab | 1 | qaylqqsgae lvrpgasvkm sckasgytft synmhwvkqt prqglewiga iypgngdtsy |
| heavy chain | 61 | nqkfkgkatl tvdkssstay mqlssltsed savyfcarvv yysnsywyfd vwgtgttvtv |
| (SEQ ID NO: | 121 | sgpsvfplap sskstsggta algclvkdyf pepvtvswns galtsgvhtf pavlqssgly |
| 26) | 181 | slssvvtvps sslgtqtyic nvnhkpsntk vdkkaepksc dkthtcppcp apellggpsv |
| | 241 | flfppkpkdt lmisrtpevt cvvvdvshed pevkfnwyvd gvevhnaktk preeqynsty |
| | 301 | rvvsvltvlh qdwlngkeyk ckvsnkalpa piektiskak gqprepqvyt lppsrdeltk |
| | 361 | nqvsltclvk gfypsdiave wesngqpenn ykttppvlds dgsfflyskl tvdksrwqqg |
| | 421 | nvfscsvmhe alhnhytqks lslspgk |
| tositumomab | 1 | qivlsqspai lsaspgekvt mtcrasssvs ymhwyqqkpg sspkpwiyap snlasgvpar |
| light chain | 61 | fsgsgsgtsy sltisrveae daatyycqqw sfnpptfgag tklelkrtva apsvfifpps |
| (SEQ ID NO: | 121 | deqlksgtas vvcllnnfyp reakvqwkvd nalqsgnsqe svteqdskds tyslsstltl |
| 27) | 181 | skadyekhkv yacevthqgl sspvtksfnr |
| rituximab | 1 | qvqlqqpgae lvkpgasvkm sckasgytft synmhwvkqt pgrglewiga iypgngdtsy |
| heavy chain | 61 | nqkfkgkatl plapssksts ggtaalgclv kdyfpepvtv yyggdwyfnv wgagttvtvs |
| (SEQ ID NO: | 121 | aastkgpsvf tadkssstay tyicnvnhkp sntkvdkkae swnsgaltsg vhtfpavlqs |
| 28) | 181 | sglyslssvv tvpssslgtq mqlssltsed savyycarst pkscdkthtc ppcpapellg |
| | 241 | gpsvflfppk pkdtlmisrt pevtcvvvdv shedpevkfn wyvdgvevhn aktkpreegy |
| | 301 | nstyrvvsvl tvlhqdwlng keykckvsnk alpapiekti skakgqprep qvytlppsrd |
| | 361 | eltknqvslt clvkgfypsd iavewesngq pennykttpp vldsdgsffl yskltvdksr |
| | 421 | wqqgnvfscs vmhealhnhy tqkslslspg k |
| rituximab | 1 | qivlsqspai lsaspgekvt mtcrasssvs yihwfqqkpg sspkpwiyat snlasgvpvr |
| light chain | 61 | fsgsgsgtsy sltisrveae daatyycqqw tsnpptfggg tkleikrtva apsvfifpps |
| (SEQ ID NO: | 121 | deqlksgtas vvcllnnfyp reakvqwkvd nalqsgnsqe svteqdskds tyslsstltl |
| 29) | 181 | skadyekhkv yacevthqgl sspvtksfnr gec |

In some embodiments, the first and/or the second antigen binding domain binds CD38. In some embodiments, the antigen binding domain is derived from a commercially available anti-CD38 antibody, CD38-binding fragments thereof, or derivative thereof, e.g., daratumumab (Darzalex®), isatuximab (Sarclisa®), and mezagitamab (TAK-079), the amino acid sequences of the heavy and light chains of which are set forth in Table 4:

TABLE 4
Amino Acid Sequences of anti-CD38 antibody fragments

| Polypeptide | Sequence | |
|---|---|---|
| daratumumab | 1 | evqllesggg lvqpggslrl scavsgftfn sfamswvrqa pgkglewvsa isgsgggtyy |
| heavy chain | 61 | adsvkgrfti srdnskntly lqmnslraed tavyfcakdk ilwfgepvfd ywgqgtlvtv |
| (SEQ ID NO: | 121 | ssastkgpsv fplapsskst sggtaalgcl vkdyfpepvt vswnsgalts gvhtfpavlq |
| 30) | 181 | ssglyslssv vtvpssslgt qtyicnvnhk psntkvdkrv epkscdktht cppcpapell |
| | 241 | ggpsvflfpp kpkdtlmisr tpevtcvvvd vshedpevkf nwyvdgvevh naktkpreeq |
| | 301 | ynstyrvvsv ltvlhqdwln gkeykckvsn kalpapiekt iskakgqpre pqvytlppsr |
| | 361 | eemtknqvsl tclvkgfyps diavewesng qpennykttp pvldsdgsff lyskltvdks |
| | 421 | rwqqgnvfsc svmhealhnh ytqkslslsp gk |
| daratumumab | 1 | eivltqspat lslspgerat lscrasqsvs sylawyqqkp gqaprlliyd asnratgipa |
| light chain | 61 | rfsgsgsgtd ftltisslep edfavyycqq rsnwpptfgq gtkveikrtv aapsvfifpp |
| (SEQ ID NO: | 121 | sdeqlksgta svvcllnnfy preakvqwkv dnalqsgnsq esvteqdskd styslsstlt |
| 31) | 181 | lskadyekhk vyacevthqg lsspvtksfn rgec |
| isatuximab | 1 | qvqlvqsgae vakpgtsvkl sckasgytft dywmqwvkqr pgqglewigt iypgdgdtgy |
| heavy chain | 61 | aqkfqgkatl tadkssktvy mhlsslased savyycargd yygsnsldyw gqgtsvtvss |
| (SEQ ID NO: | 121 | astkgpsvfp lapsskstsg gtaalgclvk dyfpepvtvs wnsgaltsgv htfpavlqss |
| 32) | 181 | glyslssvvt vpssslgtqt yicnvnhkps ntkvdkkvep kscdkthtcp pcpapellgg |
| | 241 | psvflfppkp kdtlmisrtp evtcvvvdvs hedpevkfnw yvdgvevhna ktkpreeqyn |
| | 301 | styrvvsvlt vlhqdwlngk eykckvsnka lpapiektis kakgqprepq vytlppsrde |
| | 361 | ltknqvsltc lvkgfypsdi avewesngqp ennykttppv ldsdgsffly skltvdksrw |
| | 421 | qqgnvfscsv mhealhnhyt qkslslspgk |
| isatuximab | 1 | divmtqshls mstslgdpvs itckasqdvs tvvawyqqkp gqsprrliys asyryigvpd |
| light chain | 61 | rftgsgagtd ftftissvqa edlavyycqq hysppytfgg gtkleikrtv aapsvfifpp |
| (SEQ ID NO: | 121 | sdeqlksgta svvcllnnfy preakvqwkv dnalqsgnsq esvteqdskd styslsstlt |
| 33) | 181 | lskadyekhk vyacevthqg lsspvtksfn rgec |
| mezagitamab | 1 | evqllesggg lvqpggslrl scaasgftfd dygmswvrqa pgkglewvsd iswnggkthy |
| heavy chain | 61 | vdsvkgqfti srdnskntly lqmnslraed tavyycargs lfhdssgfyf ghwgqgtlvt |
| (SEQ ID NO: | 121 | vssastkgps vfplapssks tsggtaalgc lvkdyfpepv tvswnsgalt sgvhtfpavl |
| 34) | 181 | qssglyslss vvtvpssslg tqtyicnvnh kpsntkvdkr vepkscdkth tcppcpapel |
| | 241 | lggpsvflfp pkpkdtlmis rtpevtcvvv dvshedpevk fnwyvdgvev hnaktkpree |
| | 301 | qynstyrvvs vltvlhqdwl ngkeykckvs nkalpapiek tiskakgqpr epqvytlpps |
| | 361 | reemtknqvs ltclvkgfyp sdiavewesn gqpennyktt ppvldsdgsf flyskltvdk |
| | 421 | srwqqgnvfs csvmhealhn hytqkslsls pgk |
| mezagitamab | 1 | qsvltqppsa sgtpgqrvti scsgsssnig dnyvswyqql pgtapklliy rdsqrpsgvp |
| light chain | 61 | drfsgsksgt saslaisglr sedeadyycq sydsslsgsv fgggtkltvl gqpkanptvt |
| (SEQ ID NO: | 121 | lfppsseelq ankatlvcli sdfypgavtv awkadgspvk agvettkpsk qsnnkyaass |
| 35) | 181 | ylsltpeqwk shrsyscqvt hegstvektv aptecs |

In some embodiments, the first and/or the second antigen binding domain contains a VL having the amino acid sequence set forth below (SEQ ID NO: 36):

| | |
|---|---|
| 1 | eivltqspat lslspgerat lscrasqsvs sylawyqqkp gqaprlliyd asnratgipa |
| 61 | rfsgsgsgtd ftltisslep edfavyycqq rsnwpptfgq gtkveik |

Additionally, the first and/or the second antigen binding domain contains a VH having the amino acid sequence set forth below (SEQ ID NO: 37):

| | |
|---|---|
| 1 | evqllesggg lvqpggsirl scavsgftin sfamswvrqa pgkglewvsa isgsgggtyy |
| 61 | adsvkgrfti srdnskntly lqmnslraed tavyfcakdk ilwfgepvid ywgqgtlvtv |
| 12 | ss |

In some embodiments, the first and/or the second antigen binding domain, e.g., a scFv, binds CD138. Anti-CD138 antibodies and CD138-binding fragments thereof are known in the art. See, e.g., U.S. Pat. Nos. 9,221,914, 9,387,261, 9,446,146, and 10,975,158 and U.S. Patent Application Publications 2007/0183971, 2009/0232810, 2018/0312561, 2019/0100588, 2020/0384024, and 2020/0392241.

In some embodiments, the first and/or the second antigen binding domain, e.g., a scFv, binds FCRH5. Anti-FCRH5 antibodies and FCRH5-binding fragments thereof are known in the art, e.g., cevostamab, and U.S. Pat. Nos. 8,466,260, 9,017,951, 10,323,094, 10,435,471. The amino acid sequence of a representative anti-FCRH5 heavy chain is set forth below (SEQ ID NO: 38):

| | |
|---|---|
| 1 | diqmtqspss lsasvgdrvt itckasqdvr nlvvwfqqkp gkapklliys gsyrysgvps |
| 61 | rfsgsgsgtd ftltisslqp edfatyycqq hysppytfgq gtkveikrtv aapsvfifpp |
| 121 | sdeqlksgta svvcllnnfy preakvqwkv dnalqsgnsq esvteqdskd styslsstlt |
| 181 | lskadyekhk vyacevthqg lsspvtksfn rgec |

The amino acid sequence of a representative anti-FCRH5 light chain is set forth below (SEQ ID NO: 39):

| | |
|---|---|
| 1 | evqlvesgpg lvkpsetlsl tctvsgfslt rfgvhwvrqp pgkglewlgv iwrggstdyn |
| 61 | aafvsrltis kdnsknqvsl klssvtaadt avyycsnhyy gssdyaldnw gqgtlvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkkvep kscdkthtcp pcpapellgg |
| 241 | psvflfppkp kdtlmisrtp evtcvvvdvs hedpevkfnw yvdgvevhna ktkpreegyg |
| 301 | styrvvsvlt vlhqdwlngk eykckvsnka lpapiektis kakgqprepq vytlppsree |
| 361 | mtknqvslwc lvkgfypsdi avewesngqp ennykttppv ldsdgsffly skltvdksrw |
| 421 | qqgnvfscsv mhealhnhyt qkslslspgk |

In some embodiments, the first and/or the second antigen binding domain contains a VL having the amino acid sequence set forth below (SEQ ID NO: 40):

```
  1 diqmtqspss lsasvgdrvt itckasqdvg
      iavawyqqkp gkvpklliyw astrhtgvpd
61 rfsgsgsgtd ftltisslqp edvatyycqq
      yssypytfgq gtkveik
```

In some embodiments, the first and/or the second antigen binding domain contains a VH having the amino acid sequence set forth below (SEQ ID NO: 41):

```
  1 evqlvesggg lvqpggsirl scaasgfdfs
      rywmswviga pgkglewige inpdsstiny
61 apslkdkfii srdnaknsly lqmnslraed
      tavyycarpd gnywyfdvwg qgtlvtvss
```

In some embodiments, the first and/or the second antigen binding domain, e.g., a scFv, binds GPRC5D. Anti-GPRC5D antibodies and GPRC5D-binding fragments thereof are known in the art, e.g., talquetamab, U.S. Pat. Nos. 10,562,968 and 10,590,196, and U.S. Patent Application Publications 2019/0367612, 2020/0123250, 2020/0190205, 2020/0270326, and 2021/0054094. The amino acid sequence of a representative anti-FCRH5 antibody scFv fragment is set forth below (SEQ ID NO: 42):

```
    1 divmtqtpls spvtlgqpas iscrssqslv
        hsdgntylsw lqqrpgqppr lliykisnrf
  61 fgvpdrfsgs gagtdftlki srveaedvgv
        yycmqatqfp htfgqgtkle ikggsegkss
121 gsgseskstg gsqvtlkesg pvlvkptetl
        tltctvsgfs ltnirmsvsw irqppgkale
181 wlahifsnde ksyssslksr ltisrdtsks
        qvvltltnvd pvdtatyyca rmrlpygmdv
241 wgqgttvtvs s
```

In some embodiments, the first and/or the second antigen binding domain, e.g., a scFv, binds SLAMF7. In some embodiments, the antigen binding domain is derived from a commercially available anti-SLAMF7 antibody, SLAMF7-binding fragment, or derivative thereof, e.g., elotuzumab (Empliciti®). The amino acid sequence of an elotuzumab heavy chain is set forth below (SEQ ID NO: 43):

```
    1 evqlvesggg lvqpggslrl scaasgfdfs
        rywmswvrqa pgkglewige inpdsstiny
  61 apslkdkfii srdnaknsly lqmnslraed
        tavyycarpd gnywyfdvwg qgtlvtvssa
121 stkgpsvfpl apsskstsgg taalgclvkd
        yfpepvtvsw nsgaltsgvh tfpavlqssg
181 lyslssvvtv pssslgtqty icnvnhkpsn
        tkvdkkvepk scdkthtcpp cpapellggp
241 svflfppkpk dtlmisrtpe vtcvvvdvsh
        edpevkfnwy vdgvevhnak tkpreegyns
301 tyrvvsvltv lhqdwlngke ykckvsnkal
        papiektisk akgqprepqv ytlppsrdel
361 tknqvsltcl vkgfypsdia vewesngqpe
        nnykttppvl dsdgsfflys kltvdksrwq
421 qgnvfscsvm healhnhytq kslslspgk
```

The amino acid sequence of an elotuzumab light chain is set forth below (SEQ ID NO: 44):

```
    1 diqmtqspss lsasvgdrvt itckasqdvg
        iavawyqqkp gkvpklliyw astrhtgvpd
  61 rfsgsgsgtd ftltisslqp edvatyycqq
        yssypytfgq gtkveikrtv aapsvfifpp
121 sdeqlksgta svvcllnnfy preakvqwkv
        dnalqsgnsq esvteqdskd styslsstlt
181 lskadyekhk vyacevthqg lsspvtksfn
        rgec
```

In some embodiments, the first and/or the second antigen binding domain contains a VL having the amino acid sequence set forth below (SEQ ID NO: 45):

```
1 diqmtqspss lsasvgdrvt itckasqdvg
      iavawyqqkp gkvpklliyw astrhtgvpd
61 rfsgsgsgtd ftltisslqp edvatyycqq
      yssypytfgq gtkveik
```

In some embodiments, the first and/or the second antigen binding domain contains a VH having the amino acid sequence set forth below (SEQ ID NO: 46):

```
  1 evqlvesggg lvqpggslrl scaasgfdfs
      rywmswvrqa pgkglewige inpdsstiny
61 apslkdkfii srdnaknsly lqmnslraed
      tavyycarpd gnywyfdvwg qgtlvtvss
```

Additional anti-SLAMF7 antibodies and SLAMF7-binding fragments thereof are known in the art. For example, representative antibodies and antibody scFv fragment that bind SLAMF7 include antibodies commercially available from ThermoFisher Scientific, catalog numbers 12-2229-42 (clone 162), MA5-24227 (clone 520914), CF807421 (clone OTI1F1), 57823-MSM1-PIABX (clone 3649), PA5-63125 (polyclonal), and PA5-25589 (polyclonal).

In some embodiments, the first and second antigen binding domains of the Protease CAR and the Activation CAR, respectively, bind the same TAA. In some of these embodiments, the first and second antigen binding domains have the same amino acid sequence.

The first and second transmembrane domains of the Protease CAR and the Activation CAR, respectively, connect the antigen binding domain to the intracellular domain. In some embodiments, the first and/or the second transmembrane domain is directly connected to the antigen binding domain.

In some embodiments, the first and/or second transmembrane domain is derived from CD3α, CD3β, CD3γ, CD3ζ, CD3ε, CD4, CD5, CD8α, CD9, CD16, CD22, CD28, CD33, CD37, CD45, CD64, CD80, CD86, CD134, CD154, 4-1BB (also known CD137 or TNF Receptor Superfamily Member 9 (TNFRSF9)), FcεRIα, FcεRIβ, FcεRIγ, ICOS, KIR2DS2, MHC class I, MHC class II, or NKG2D. In some embodiments, the transmembrane domain is derived from CD3ζ, CD4, CD8α, CD28, or CD137 (4-1BB). Amino acid sequences of representative transmembrane domains are listed in Table 5:

TABLE 5
Amino Acid Sequences of Transmembrane
domains

| Transmembrane domain | Sequence |
|---|---|
| CD3ζ (SEQ ID NO: 47) | LCYLLDGILFIYGVILTAL |
| | FL |
| CD4 (SEQ ID NO: 48) | MALIVLGGVAGLLLFIGLG |
| | IFF |
| CD8a (SEQ ID NO: 49) | IYIWAPLAGTCGVLLLSLV |
| | ITLYC |
| CD28 (SEQ ID NO: 50) | FWVLVVVGGVLACYSLLVT |
| | VAFIIFWV |
| CD137 (4-1BB) | IISFFLALTSTALLFLLF |
| (SEQ ID NO: 51) | FLTLRFSVV |

The amino acid sequence of a naturally occurring transmembrane domain may be modified by an amino acid substitution to avoid binding of such regions to the transmembrane domain of the same or different surface membrane proteins to minimize interactions with other members of a receptor complex. See, e.g., U.S. Patent Application Publication 2021/0101954; Soudais et al., Nat Genet 3:77-81 (1993); Muller et al., Front. Immunol. 12:639818-13 (2021); and Elazar et al., elife 11: e75660-29 (2022).

In some embodiments, the Protease CAR, the Activator CAR, or both CARs further include a hinge domain disposed between the antigen binding domain and the transmembrane domain. A hinge domain may provide flexibility in terms of allowing the antigen binding domain to obtain an optimal orientation for antigen-binding, thereby enhancing antitumor activities of the cell expressing the CAR. The hinge domains of the Protease CAR and the Activator CAR, which are also referred to as the first and second hinge domains, respectively, may be the same or different.

In some embodiments, the first and/or second hinge domain is derived from IgA, IgD, IgE, IgG, or IgM. In some embodiments, the first and/or the second hinge domain is derived from CD35, CD4, CD8α, CD28, IgG1, IgG2, or IgG4. Amino acid sequences of representative hinge domains are listed in Table 6:

TABLE 6
Amino Acid Sequences of Hinge domains

| Hinge domain | Sequence |
|---|---|
| CD3ζ (SEQ ID NO: 52) | QSFGLLDPK |
| CD4 (SEQ ID NO: 53) | LSEGDKVKMDSRIQVLSRGVNQT |
| CD8α (SEQ ID NO: 54) | KPTTTPAPRPPTPAPTIASQPLS |
| | KRPEACRPAAGGAVHTRGLDFAC |
| | DIY |
| CD2ϵ (SEQ ID NO: 55) | IEVMYPPPYLDNERSNGTIIHVK |
| | GKHLCPSPLFPGPSKP |
| IgG1 (SEQ ID NO: 56) | EPKSCDKTHTCPPCPAPELLGG |
| IgG2 (SEQ ID NO: 57) | ERKCCVECPPCPAPPAAA |
| IgG4 (SEQ ID NO: 58) | ESKYGPPCPPCPAPEFLGG |

In some embodiments, the intracellular domain of the Protease CAR, the Activation CAR, or both the Protease CAR and the Activation CAR, which are also referred to herein as the first and second intracellular domains, respectively, contain a signaling domain that enables intracellular signaling and immune cell function. The signaling domain may include a primary signaling domain and/or a co-stimulatory signaling domain. In some embodiments, the intracellular domain is capable of delivering a signal approximating that of natural ligation of an ITAM-containing molecule or receptor complex such as a TCR receptor complex. The signaling domains that may be present in the first and second intracellular domains may be the same or different. Therefore, in some embodiments, the first and second intracellular domains may the same primary signaling domains and different co-stimulatory domains, or vice versa.

In some embodiments, the first and/or second intracellular signaling domain includes a plurality, e.g., 2 or 3, co-stimulatory signaling domains described herein, e.g., selected from 4-1BB, CD35, CD28, CD27, ICOS, and OX40. In some embodiments, the intracellular signaling domain may include a CD3 (domain as a primary signaling domain, and any of the following pairs of co-stimulatory signaling domains from the extracellular to the intracellular direction, namely: 4-1BB-CD27; CD27-4-1BB; 4-1BB-CD28; CD28-4-1BB; 4-1BB-CD3ζ; CD3ζ-4-1BB; CD28-CD3ζ; CD3ζ-CD28; OX40-CD28 and CD28-OX40. In some embodiments the primary signaling domain is derived from CD3ζ, CD27, CD28, CD40, KIR2DS2, MyD88, or OX40. In some embodiments, the co-stimulatory signaling domain is derived from one or more of CD3γ, CD3δ, CD3ε, CD3ζ, CD27, CD40, CD28, CD72, CD80, CD86, CLEC-1, 4-1BB, TYROBP (DAP12), Dectin-1, FcαRI, FcγRI, FcγRII, FcγRIII, FcεRI, IL-2RB, ICOS, KIR2DS2, MyD88, OX40, and ZAP70. Amino acid sequence of representative signaling domains are listed in Table 7.

TABLE 7
Amino Acid Sequences of Signaling domains

| Signaling domain | Sequence |
|---|---|
| CD3ζ (SEQ ID NO: 59) | RVKFSRSADAPAYKQGQNQLYNELNLGRREEYDVLDKRRGRDPEMG |
| | GKPRRKNPQEGLYNELQKDKMAEAYSEIGMKGERRRGKGHDGLYQ |
| | GLSTATKDTYDALHMQALPPR |
| CD3ζ variant | RVKFSRSADAPAYQQGQNQLYNELNLGRREEYDVLDKRRGRDPEMG |
| (SEQ ID NO: 60) | GKPRRKNPQEGLYNELQKDKMAEAYSEIGMKGERRRGKGHDGLYQ |
| | GLSTATKDTYDALHMQALPPR |
| CD3ϵ (SEQ ID NO: 61) | KNRKAKAKPVTRGAGAGGRQRGQNKERPPPVPNPDYEPIRKGQRDL |
| | YSGLNQRRI |
| CD4 (SEQ ID NO: 62) | RSKRSRLLHSDYMNMTPRRPGPTRKHYQPYAPPRDFAAYRS |
| CD27 (SEQ ID NO: 63) | QRRKYRSNKGESPVEPAEPCHYSCPREEEGSTIPIQEDYRKPEPAC |
| | SP |
| CD28 (SEQ ID NO: 64) | RSKRSRLLHSDYMNMTPRRPGPTRKHYQPYAPPRDFAAYRS |
| CD40 (SEQ ID NO: 65) | MIETYNQTSPRSAATGLPISMK |
| CD80 (SEQ ID NO: 66) | TYCFAPRCRERRRNERLRRESVRPV |
| CD86 (SEQ ID NO: 67) | KWKKKKRPRNSYKCGTNTMEREESEQTKKREKIHIPERSDEAQRVF |
| | KSSKTSSCDKSDTCF |
| CD137 (4-1BB) (SEQ ID | KRGRKKLLYIFKQPFMRPVQTTQEEDGCSCRFPEEEEGGCEL |
| NO: 68) | |
| DAP10 (SEQ ID NO: 69) | CWLTKKKYSSSVHDPNGEYMFMRAVNTAKKSRLTDVTL |
| DAP12 (SEQ ID NO: 70) | YFLGRLVPRGRGAAEAATRKQRITETESPYQELQGQRSDVYSDLNT |
| | QRPYYK |
| FcϵRI (SEQ ID NO: 71) | RLKIQVRKAAITSYEKSDGVYTGLSTRNQETYETLKHEKPPQ |
| ICOS (SEQ ID NO: 72) | RSKRSRLLHSDYMNMTPRRPGPTRKHYQPYAPPRDFAAYRS |
| KIR2DS2 (SEQ ID NO: 73) | HRWCSNKKNAAVMDQEPAGNRTVNSEDSDEQDHQEVSYA |
| MyD88 (SEQ ID NO: 74) | MAAGGPGAGSAAPVSSTSSLPLAALNMRVRRRLSLFLNVRTQVAAD |
| | WTALAEEMDFEYLEIRQLETQADPTGRLLDAWQGRPGASVGRLLEL |
| | LTKLGRDDVLLELGPSIEEDCQKYILKQQQEEAEKPLQVAAVDSSV |
| | PRTAELAGITTLDDPLGHMPERFDAFICYCPSDIQFVQEMIRQLEQ |
| | TNYRLKLCVSDRDVLPGTCVWSIASELIEKRCRRMVVVVSDDYLQS |
| | KECDFQTKFALSLSPGAHQKRLIPIKYKAMKKEFPSILRFITVCDY |
| | TNPCTKSWFWTRLAKALSLP |
| OX40 (SEQ ID NO: 75) | ALYLLRRDQRLPPDAHKPPGGGSFRTPIQEEQADAHSTLAKI |
| ZAP70 (SEQ ID NO: 76) | MPDPAAHLPFFYGSISRAEAEEHLKLAGMADGLFLLRQCLRSLGGY |
| | VLSLVHDVRFHHFPIERQLNGTYAIAGGKAHCGPAELCEFYSRDPD |
| | GLPCNLRKPCNRPSGLEPQPGVFDCLRDAMVRDYVRQTWKLEGEAL |
| | EQAIISQAPQVEKLIATTAHERMPWYHSSLTREEAERKLYSGAQTD |
| | GKFLLRPRKEQGTYALSLIYGKTVYHYLISQDKAGKYCIPEGTKFD |
| | TLWQLVEYLKLKADGLIYCLKEACPNSSASNASGAAAPTLPAHPST |
| | LTHPQRRIDTLNSDGYTPEPARITSPDKPRPMPMDTSVYESPYSDP |
| | EELKDKKLFLKRDNLLIADIELGCGNFGSVRQGVYRMRKKQIDVAI |
| | KVLKQGTEKADTEEMMREAQIMHOLDNPYIVRLIGVCQAEALMLVM |
| | EMAGGGPLHKFLVGKREEIPVSNVAELLHQVSMGMKYLEEKNFVHR |
| | DLAARNVLLVNRHYAKISDFGLSKALGADDSYYTARSAGKWPLKWY |
| | APECINFRKFSSRSDVWSYGVTMWEALSYGQKPYKKMKGPEVMAFI |
| | EQGKRMECPPECPPELYALMSDCWIYKWEDRPDFLTVEQRMRACYY |
| | SLASKVEGPPGSTQKAEAACA |

In some embodiments, the primary signaling domain is derived from CD28 and the co-stimulatory domain is derived from 4-1BB. In some embodiments, the primary signaling domain is derived from CD28 and the co-stimulatory domain is derived from CD3ζ. In some embodiments, the primary signaling domain is derived from CD28 and the co-stimulatory domain is derived from 4-1BB and CD3ζ.

Amino acid sequences of additional isoforms of CD28 are provided in Table 8.

TABLE 8
Amino Acid Sequences of CD28 isoforms

| Isoform | Sequence |
|---|---|
| CD28 isoform 1 | MLRLLLALNLFPSIQVTGNKILVKQSPMLVAYDNAVNLSCKYSYNL |
| NP_006130.1 | FSREFRASLHKGLDSAVEVCVVYGNYSQQLQVYSKTGFNCDGKLG |
| (SEQ ID NO: 77) | NESVTFYLQNLYVNQTDIYFCKIEVMYPPPYLDNEKSNGTIIHVKGK |
| | HLCPSPLFPGPSKPFWVLVVVGGVLACYSLLVTVAFIIFWVRSKRSR |
| | LLHSDYMNMTPRRPGPTRKHYQPYAPPRDFAAYRS |
| CD28 isoform 2 | MLRLLLALNLFPSIQVTGNKILVKQSPMLVAYDNAVNLSWKHLCPS |
| NP_001230006.1 | PLFPGPSKPFWVLVVVGGVLACYS |
| (SEQ ID NO: 78) | LLVTVAFIIFWVRSKRSRLLHSDYMNMTPRRPGPTRKHYQPYAPPR |
| | DFAAYRS |
| CD28 isoform 3 | MLRLLLALNLFPSIQVTGKHLCPSPLFPGPSKPFWVLVVVGGVLACY |
| NP_001230007.1 | SLLVTVAFIIFWVRSKRSRLLHSDYMNMTPRRPGPTRKHYQPYAPPR |
| (SEQ ID NO: 79) | DFAAYRS |
| CD28 isoform 4 | MPCGLSALIMCPKGMVAVVVAVDDGDSQALAGNKILVKQSPMLVA |
| NP_001397910.1 | YDNAVNLSCKYSYNLFSREFRASLHKGLDSAVEVCVVYGNYSQQL |
| (SEQ ID NO: 80) | QVYSKTGFNCDGKLGNESVTFYLQNLYVNQTDIYFCKIEVMYPPP |
| | YLDNEKSNGTIIHVKGKHLCPSPLFPGPSKPFWVLVVVGGVLACY |
| | SLLVTVAFIIFWVRSKRSRLLHSDYMNMTPRRPGPTRKHYQPYAP |
| | PRDFAAYRS |

The intracellular domain of the Protease CAR contains a protease domain. In some embodiments, the protease is derived from the Tobacco Etch Virus (TEV) protease (TEVp), the NEDP1 protease, a calpain protease, or a SUMO protease. TEVp (Enzyme Commission number 3.4.22.44, also known as TEV nuclear-inclusion-a endopeptidase) is a catalytically active 27 kDa C-terminal domain of the nuclear inclusion a protease. TEVp is a highly sequence-specific cysteine protease (Dougherty et al., Virology 172 (1): 302-10 (1989)). The amino acid sequence of a representative TEVp is set forth below (SEQ ID NO: 81):

```
    1 geslfkgprd ynpisstich ltnesdghtt
        slygigfgpf iitnkhlfrr nngtllvqsl
  61 hgvfkvkntt tlqqhlidgr dmiiirmpkd
        fppfpqklkf repqreeric lvttnfqtks
121 mssmvsdtsc tipssdgifw khwiqtkdgq
        cgsplvstrd gfivgihsas nftntnnyft
181 svpknfmell tngeaqqwvs gwilnadsvl
        wgghkvfmv
```

TEVp recognizes a cleavage site. TEVp recognizes the seven-residue target amino acid sequence ENLYFQX (SEQ ID NO: 82), where X is M, G, or S. The cleaved peptide bond is between Q and X. In some embodiments, the cleavage site has the amino acid sequence ENLYFQM (SEQ ID NO: 83).

Calpain proteases are known in the art. See, e.g., U.S. Pat. Nos. 7,001,907 and 9,833,498. The NEDP1 protease is known in the art. See, e.g., U.S. Pat. Nos. 7,842,460, 8,642,256, and 10,466,249. SUMO proteases are known in the art. See, e.g., U.S. Pat. Nos. 7,750,134, 8,119,369, 10,767,185, and 11,261,437. Other enzymatic cleavage systems or transcriptional systems have been reported and are within the scope of this disclosure as alternatives or as additional “on”-switches See, e.g., Barnea et al., Proc. Natl. Acad. Sci. U.S.A. 105 (1): 64-9 (2008), and Morsut et al., Cell 164 (4): 780-91 (2016).

In some embodiments, the Protease CAR has the nucleic acid sequence set forth below (SEQ ID NO: 84), and which contains the features set forth in Table 9, and which may be incorporated into a pLVC-CMV 100 construct background:

| | |
|---|---|
| 1 | tggaagggct aattcactcc caaagaagac aagatatcct tgatctgtgg atctaccaca |
| 61 | cacaaggcta cttccctgat tagcagaact acacaccagg gccaggggtc agatatccac |
| 121 | tgacctttgg atggtgctac aagctagtac cagttgagcc agataaggta gaagaggcca |
| 181 | ataaaggaga gaacaccagc ttgttacacc ctgtgagcct gcatgggatg gatgacccgg |
| 241 | agagagaagt gttagagtgg aggtttgaca gccgcctagc atttcatcac gtggcccgag |
| 301 | agctgcatcc ggagtacttc aagaactgct gatatcgagc ttgctacaag ggactttccg |
| 361 | ctggggactt tccagggagg cgtggcctgg gcgggactgg ggagtggcga gccctcagat |
| 421 | cctgcatata agcagctgct ttttgcctgt actgggtctc tctggttaga ccagatctga |
| 481 | gcctgggagc tctctggcta actagggaac ccactgctta agcctcaata aagcttgcct |
| 541 | tgagtgcttc aagtagtgtg tgcccgtctg ttgtgtgact ctggtaacta gagatccctc |
| 601 | agaccctttt agtcagtgtg gaaaatctct agcagtggcg cccgaacagg gacttgaaag |
| 661 | cgaaagggaa accagaggag ctctctcgac gcaggactcg gcttgctgaa gcgcgcacgg |
| 721 | caagaggcga ggggcggcga ctggtgagta cgccaaaaat tttgactagc ggaggctaga |
| 781 | aggagagaga tgggtgcgag agcgtcagta ttaagcgggg gagaattaga tcgcgatggg |
| 841 | aaaaaattcg gttaaggcca gggggaaaga aaaaatataa attaaaacat atagtatggg |
| 901 | caagcaggga gctagaacga ttcgcagtta atcctggcct gttagaaaca tcagaaggct |
| 961 | gtagacaaat actgggacag ctacaaccat cccttcagac aggatcagaa gaacttagat |
| 1021 | cattatataa tacagtagca accctctatt gtgtgcatca aaggatagag ataaaagaca |
| 1081 | ccaaggaagc tttagacaag atagaggaag agcaaaacaa aagtaagacc accgcacagc |
| 1141 | aagcggccgg ccgctgatct tcagacctgg aggaggagat atgagggaca attggagaag |
| 1201 | tgaattatat aaatataaag tagtaaaaat tgaaccatta ggagtagcac ccaccaaggc |
| 1261 | aaagagaaga gtggtgcaga gagaaaaaag agcagtggga ataggagctt tgttccttgg |
| 1321 | gttcttggga gcagcaggaa gcactatggg cgcagcgtca atgacgctga cggtacaggc |
| 1381 | cagacaatta ttgtctggta tagtgcagca gcagaacaat ttgctgaggg ctattgaggc |
| 1441 | gcaacagcat ctgttgcaac tcacagtctg gggcatcaag cagctccagg caagaatcct |
| 1501 | ggctgtggaa agatacctaa aggatcaaca gctcctgggg atttggggtt gctctggaaa |
| 1561 | actcatttgc accactgctg tgccttggaa tgctagttgg agtaataaat ctctggaaca |
| 1621 | gatttggaat cacacgacct ggatggagtg ggacagagaa attaacaatt acacaagctt |
| 1681 | aatacactcc ttaattgaag aatcgcaaaa ccagcaagaa aagaatgaac aagaattatt |
| 1741 | ggaattagat aaatgggcaa gtttgtggaa ttggtttaac ataacaaatt ggctgtggta |
| 1801 | tataaaatta ttcataatga tagtaggagg cttggtaggt ttaagaatag tttttgctgt |
| 1861 | actttctata gtgaatagag ttaggcaggg atattcacca ttatcgtttc agacccacct |
| 1921 | cccaaccccg aggggacccg acaggcccga aggaatagaa gaagaaggtg gagagagaga |
| 1981 | cagagacaga tccattcgat tagtgaacgg atctcgacgg tatcgccttt aaaagaaaag |
| 2041 | gggggattgg ggggtacagt gcaggggaaa gaatagtaga cataatagca acagacatac |
| 2101 | aaactaaaga attacaaaaa caaattacaa aaattcaaaa ttttcgggtt tattacaggg |
| 2161 | acagcagaga tccagtttat cgatgagtaa ttcatacaaa aggactcgcc cctgccttgg |
| 2221 | ggaatcccag ggaccgtcgt taaactccca ctaacgtaga acccagagat cgctgcgttc |
| 2281 | ccgccccctc acccgcccgc tctcgtcatc actgaggtgg agaagagcat gcgtgaggct |
| 2341 | ccggtgcccg tcagtgggca gagcgcacat cgcccacagt ccccgagaag ttggggggag |
| 2401 | gggtcggcaa ttgaaccggt gcctagagaa ggtggcgcgg ggtaaactgg gaaagtgatg |
| 2461 | tcgtgtactg gctccgcctt tttcccgagg gtgggggaga accgtatata agtgcagtag |
| 2521 | tcgccgtgaa cgttcttttt cgcaacgggt ttgccgccag aacacaggta agtgccgtgt |
| 2581 | gtggttcccg cgggcctggc ctctttacgg gttatggccc ttgcgtgcct tgaattactt |
| 2641 | ccacgcccct ggctgcagta cgtgattctt gatcccgagc ttcgggttgg aagtgggtgg |
| 2701 | gagagttcga ggccttgcgc ttaaggagcc ccttcgcctc gtgcttgagt tgaggcctgg |
| 2761 | cctgggcgct ggggccgccg cgtgcgaatc tggtggcacc ttcgcgcctg tctcgctgct |
| 2821 | ttcgataagt ctctagccat ttaaaatttt tgatgacctg ctgcgacgct ttttttctgg |
| 2881 | caagatagtc ttgtaaatgc gggccaagat ctgcacactg gtatttcggt ttttggggcc |
| 2941 | gcgggcggcg acggggcccg tgcgtcccag cgcacatgtt cggcgaggcg gggcctgcga |
| 3001 | gcgcggccac cgagaatcgg acgggggtag tctcaagctg gccggcctgc tctggtgcct |
| 3061 | ggcctcgcgc cgccgtgtat cgccccgccc tgggcggcaa ggctggcccg gtcggcacca |
| 3121 | gttgcgtgag cggaaagatg gccgcttccc ggccctgctg cagggagctc aaaatggagg |
| 3181 | acgcggcgct cgggagagcg ggcgggtgag tcacccacac aaaggaaaag ggcctttccg |
| 3241 | tcctcagccg tcgcttcatg tgactccacg gagtaccggg cgccgtccag gcacctcgat |
| 3301 | tagttctcga gcttttggag tacgtcgtct ttaggttggg gggaggggtt ttatgcgatg |
| 3361 | gagtttcccc acactgagtg ggtggagact gaagttaggc cagcttggca cttgatgtaa |
| 3421 | ttctccttgg aatttgccct ttttgagttt ggatcttggt tcattctcaa gcctcagaca |
| 3481 | gtggttcaaa gtttttttct tccatttcag gtgtcgtgat tcgaattcgc cgccaccatg |
| 3541 | gccttaccag tgaccgcctt gctcctgccg ctggccttgc tgctccacgc cgccaggccg |
| 3601 | gagcagaagc tcatctccga ggaggacctg gatatccaga tgacccagag cccgtcgagc |
| 3661 | ctttccgcct ccgtggggga cagggtcact atcacctgct ctgcttccca ggacatctct |
| 3721 | aactacctga attggtacca gcagaagccc ggcaaggcgc ccaagctact catttactac |
| 3781 | accagcaacc tgcactctgg tgtgcctagc cgcttttcag gttccggctc cggcaccgac |
| 3841 | ttcaccctga ctatttcgag tttgcagcca gaggacttcg ccacctacta ttgtcaacag |
| 3901 | taccgcaagc tgccgtggac cttcggacag ggcacaaaac tggagatcaa gggtggcggt |
| 3961 | ggctcgggcg gtggtgggtc gggtggcggc ggatctcaag tgcagctggt gcagagcggg |
| 4021 | gcagaagtca agaagcctgg ctcctctgta aaggtgtcat gcaaggcttc cggtggcacg |
| 4081 | ttcagcaact attggatgca ttgggtccgc caggcccccg gacagggcct ggagtggatg |
| 4141 | ggggccacct accgtggcca cagcgatact tactacaacc agaaatttaa aggccgcgtg |
| 4201 | accatcaccg cggacaagtc gacctccaca gcctacatgg agctgtctag tttgcgctcg |
| 4261 | gaggacactg ctgtttatta ctgtgcgcgg ggtgccattt acgacggcta cgatgtgctc |
| 4321 | gacaattggg gccagggaac ccttgtcacc gtgtcctcta ccacgacgcc agcgccgcga |
| 4381 | ccaccaacac cggcgcccac catcgcgtcg cagcccctgt ccctgcgccc agaggcgtgc |
| 4441 | cggccagcgg cggggggcgc agtgcacacg agggggctgg acttcgccty tgatttttgg |
| 4501 | gtgctggtgg tggttggtgg agtcctggct tgctatagct tgctagtaac agtggccttt |
| 4561 | attattttct gggtgaggag taagaggagc aggctcctgc acagtgacta catgaacatg |
| 4621 | actccccgcc gccccgggcc cacccgcaag cattaccagc cctatgcccc accacgcgac |
| 4681 | ttcgcagcct atcgctccaa acggggcaga aagaaactcc tgtatatatt caaacaacca |
| 4741 | tttatgagac cagtacaaac tactcaagag gaagatggct gtagctgccg atttccagaa |
| 4801 | gaagaagaag gaggatgtga actgagagtg aagttcagca ggagcgcaga cgcccccgcg |
| 4861 | taccagcagg gccagaacca gctctataac gagctcaatc taggacgaag agaggagtac |
| 4921 | gatgttttgg acaagagacg tggccgggac cctgagatgg ggggaaagcc gagaaggaag |
| 4981 | aaccctcagg aaggcctgta caatgaactg cagaaagata agatggcgga ggcctacagt |
| 5041 | gagattggga tgaaaggcga gcgccggagg ggcaaggggc acgatggcct ttaccagggt |
| 5101 | ctcagtacag ccaccaagga cacctacgac gcccttcaca tgcaggccct gccccctcgc |
| 5161 | ggtggcggct ccggggagag cctgttcaag ggccctcgtg actacaatcc aatttcgtcg |
| 5221 | accatctgtc acttgacaaa cgagtccgat ggtcatacca cttctctgta cggcatcggt |
| 5281 | ttcggaccct tcattataac caacaagcac ttgttccgcc gcaacaacgg caccctgctt |
| 5341 | gtgcagagcc tacatggagt ttttaaggtc aaaaacacga ccactctgca acagcacctg |
| 5401 | attgacggcc gggacatgat catcatccgt atgcccaagg acttcccccc gtttcctcag |
| 5461 | aagctcaaat ttcgcgagcc tcagagggag gagcgcatct gtctggtcac cacaaatttc |
| 5521 | cagaccaagt ctatgtcttc catggtgagt gacacctcat gcactttccc gtcttccgat |
| 5581 | ggtatcttct ggaagcactg gatccagacg aaagatggac agtgcgggtc ccccctggtg |
| 5641 | tccactcgcg acggcttcat cgtgggcatc cactcggcct caaatttcac caacacgaac |
| 5701 | aactatttca cctccgtgcc aaagaacttt atggaactgc tgaccaacca ggaggctcag |
| 5761 | cagtgggtca gcggctggcg actcaacgcg gacagcgtac tttggggcgg gcacaaggtg |
| 5821 | ttcatggtgt aataacatat gcctaggtct agaacgcgtc tggaacaatc aacctctgga |
| 5881 | ttacaaaatt tgtgaaagat tgactggtat tcttaactat gttgctcctt ttacgctatg |
| 5941 | tggatacgct gctttaatgc ctttgtatca tgctattgct tcccgtatgg ctttcatttt |
| 6001 | ctcctccttg tataaatcct ggttgctgtc tctttatgag gagttgtggc ccgttgtcag |
| 6061 | gcaacgtggc gtggtgtgca ctgtgtttgc tgacgcaacc cccactggtt ggggcattgc |
| 6121 | caccacctgt cagctccttt ccgggacttt cgctttcccc ctccctattg ccacggcgga |
| 6181 | actcatcgcc gcctgccttg cccgctgctg gacaggggct cggctgttgg gcactgacaa |
| 6241 | ttccgtggtg ttgtcgggga agctgacgtc ctttccatgg ctgctcgcct gtgttgccac |
| 6301 | ctggattctg cgcgggacgt ccttctgcta cgtcccttcg gccctcaatc cagcggacct |
| 6361 | tccttcccgc ggcctgctgc cggctctgcg gcctcttccg cgtcttcgcc ttcgccctca |
| 6421 | gacgagtcgg atctcccttt gggccgcctc cccgcctgga attaattctg cagtcgagac |
| 6481 | ctagaaaaac atggagcaat cacaagtagc aatacagcag ctaccaatgc tgattgtgcc |
| 6541 | tggctagaag cacaagagga ggaggaggtg ggttttccag tcacacctca ggtaccttta |
| 6601 | agaccaatga cttacaaggc agctgtagat cttagccact ttttaaaaga aaagagggga |
| 6661 | ctggaagggc taattcactc ccaacgaaga caagatatcc ttgatctgtg gatctaccac |
| 6721 | acacaaggct acttccctga ttagcagaac tacacaccag ggccaggggt cagatatcca |
| 6781 | ctgacctttg gatggtgcta caagctagta ccagttgagc cagataaggt agaagaggcc |
| 6841 | aataaaggag agaacaccag cttgttacac cctgtgagcc tgcatgggat ggatgacccg |
| 6901 | gagagagaag tgttagagtg gaggtttgac agccgcctag catttcatca cgtggcccga |
| 6961 | gagctgcatc cggagtactt caagaactgc tgatatcgag cttgctacaa gggactttcc |
| 7021 | gctggggact ttccagggag gcgtggcctg ggcgggactg gggagtggcg agccctcaga |
| 7081 | tcctgcatat aagcagctgc tttttgcctg tactgggtct ctctggttag accagatctg |
| 7141 | agcctgggag ctctctggct aactagggaa cccactgctt aagcctcaat aaagcttgcc |
| 7201 | ttgagtgctt caagtagtgt gtgcccgtct gttgtgtgac tctggtaact agagatccct |
| 7261 | cagacccttt tagtcagtgt ggaaaatctc tagcagtagt agttcatgtc atcttattat |
| 7321 | tcagtattta taacttgcaa agaaatgaat atcagagagt gagaggcctt gacattgcta |
| 7381 | gcgtttaccg tcgacctcta gctagagctt ggcgtaatca tggtcatagc tgtttcctgt |
| 7441 | gtgaaattgt tatccgctca caattccaca caacatacga gccggaagca taaagtgtaa |
| 7501 | agcctggggt gcctaatgag tgagctaact cacattaatt gcgttgcgct cactgcccgc |
| 7561 | tttccagtcg ggaaacctgt cgtgccagct gcattaatga atcggccaac gcgcggggag |
| 7621 | aggcggtttg cgtattgggc gctcttccgc ttcctcgctc actgactcgc tgcgctcggt |
| 7681 | cgttcggctg cggcgagcgg tatcagctca ctcaaaggcg gtaatacggt tatccacaga |
| 7741 | atcaggggat aacgcaggaa agaacatgtg agcaaaaggc cagcaaaagg ccaggaaccg |
| 7801 | taaaaaggcc gcgttgctgg cgtttttcca taggctccgc ccccctgacg agcatcacaa |
| 7861 | aaatcgacgc tcaagtcaga ggtggcgaaa cccgacagga ctataaagat accaggcgtt |
| 7921 | tccccctgga agctccctcg tgcgctctcc tgttccgacc ctgccgctta ccggatacct |
| 7981 | gtccgccttt ctcccttcgg gaagcgtggc gctttctcat agctcacgct gtaggtatct |
| 8041 | cagttcggtg taggtcgttc gctccaagct gggctgtgtg cacgaacccc ccgttcagcc |
| 8101 | cgaccgctgc gccttatccg gtaactatcg tcttgagtcc aacccggtaa gacacgactt |
| 8161 | atcgccactg gcagcagcca ctggtaacag gattagcaga gcgaggtatg taggcggtgc |
| 8221 | tacagagttc ttgaagtggt ggcctaacta cggctacact agaagaacag tatttggtat |
| 8281 | ctgcgctctg ctgaagccag ttaccttcgg aaaaagagtt ggtagctctt gatccggcaa |
| 8341 | acaaaccacc gctggtagcg gtggtttttt ttttgcaag cagcagatta cgcgcagaaa |
| 8401 | aaaaggatct caagaagatc ctttgatctt tictacgggg tctgacgctc agtggaacga |
| 8461 | aaactcacgt taagggattt tggtcatgag attatcaaaa aggatcttca cctagatcct |
| 8521 | tttaaattaa aaatgaagtt ttaaatcaat ctaaagtata tatgagtaaa cttggtctga |
| 8581 | cagttaccaa tgcttaatca gtgaggcacc tatctcagcg atctgtctat ttcgttcatc |
| 8641 | catagttgcc tgactccccg tcgtgtagat aactacgata cgggagggct taccatctgg |
| 8701 | ccccagtgct gcaatgatac cgcgagaccc acgctcaccg gctccagatt tatcagcaat |
| 8761 | aaaccagcca gccggaaggg ccgagcgcag aagtggtcct gcaactttat ccgcctccat |
| 8821 | ccagtctatt aattgttgcc gggaagctag agtaagtagt tcgccagtta atagtttgcg |
| 8881 | caacgttgtt gccattgcta caggcatcgt ggtgtcacgc tcgtcgtttg gtatggcttc |
| 8941 | attcagctcc ggttcccaac gatcaaggcg agttacatga tcccccatgt tgtgcaaaaa |
| 9001 | agcggttagc tccttcggtc ctccgatcgt tgtcagaagt aagttggccg cagtgttatc |
| 9061 | actcatggtt atggcagcac tgcataattc tcttactgtc atgccatccg taagatgctt |
| 9121 | ttctgtgact ggtgagtact caaccaagtc attctgagaa tagtgtatgc ggcgaccgag |
| 9181 | ttgctcttgc ccggcgtcaa tacgggataa taccgcgcca catagcagaa ctttaaaagt |
| 9241 | gctcatcatt ggaaaacgtt cttcggggcg aaaactctca aggatcttac cgctgttgag |
| 9301 | atccagttcg atgtaaccca ctcgtgcacc caactgatct tcagcatctt ttactttcac |
| 9361 | cagcgtttct gggtgagcaa aaacaggaag gcaaaatgcc gcaaaaaagg gaataagggc |
| 9421 | gacacggaaa tgttgaatac tcatactctt cctttttcaa tattattgaa gcatttatca |
| 9481 | gggttattgt ctcatgagcg gatacatatt tgaatgtatt tagaaaaata aacaaatagg |
| 9541 | ggttccgcgc acatttcccc gaaaagtgcc acctgacgtc gacggatcgg gagatcaact |
| 9601 | tgtttattgc agcttataat gattacaaat aaagcaatag catcacaaat ttcacaaata |
| 9661 | aagcattttt ttcactgcat tctagttgtg gtttgtccaa actcatcaat gtatcttatc |
| 9721 | atgtctggat caactggata actcaagcta accaaaatca tcccaaactt cccaccccat |
| 9781 | accctattac cactgccaat tacctgtggt ttcatttact ctaaacctgt gattcctctg |
| 9841 | aattattttc attttaaaga aattgtattt gttaaatatg tactacaaac ttagtagt |

TABLE 9
Protease CAR nucleic acid construct

| | Length | |
|---|---|---|
| Name | (bp) | Reference |
| pLVX-CMV 100 vector | 2178 | Dean et al., Biophys. J. 110(6): |
| backbone | | 1456-65 (2016) |
| multiple cloning site | 6 | |
| EF-1α promoter | 1335 | Human elongation factor EF-1- |
| | | alpha gene, NCBI Accession |
| | | No. J04617.1 |
| multiple cloning site | 12 | |
| Kozak sequence | 6 | Addgene plasmid # 60360 |
| Human CD8A transcript | 63 | Homo sapiens CD8α molecule |
| | | (CD8A), transcript variant 1, |
| | | mRNA/A |
| | | NCBI Accession No. NM_001768.6 |
| Myc Tag (EQKLISEEDL | 30 | Roybal et al., Cell 164(4): 770-9 |
| (SEQ ID NO: 85)) | | (2016) |
| VL of anti-tumor protein A | 321 | 10797L\|belantamab\|Humanized∥ |
| | | L-KAPPA (V-KAPPA (1-107) [D1] |
| | | from IMGT |
| Linker_(G4S)3 | 45 | Synthetic construct anti-tumor |
| | | ScFv antibody gene. NCBI |
| | | Accession No. AF363774.1 |
| VH of anti-tumor protein A | 363 | 10797H\|Humanized∥H-GAMMA-1 |
| | | (1-121) [D1] from IMGT |
| Hinge | 135 | Homo sapiens CD8A, transcript |
| | | variant 1. NCBI Accession No. |
| | | NM_001768.6 |
| CD28 Transmembrane | 204 | Homo sapiens CD28 molecule |
| Intracellular | | (CD28), transcript variant 1. NCBI |
| | | Accession No. NM_006139.4 |
| 4-1BB_Cytoplasmic | 126 | Homo sapiens TNF receptor |
| domain | | superfamily member 9 (TNFRSF9). |
| | | NCBI Accession No. NM_001561.5. |
| CD3ξ_Intracellular region | 336 | Homo sapiens CD247 molecule |
| | | (CD247), transcript variant 2. |
| | | NCBI Accession No. NM_000734.4 |
| GGGS Linker | 12 | Wang W., et., al., Nat Biotechnol. |
| (SEQ ID NO: 90) | | 2017 Sep; 35(9): 864-871. doi: |
| | | 10.1038/nbt.3909. Epub 2017 |
| | | Jun 26. |
| delta220-242, S219V | 657 | Wang W., et., at., Nat Biotechnol. |
| TEVprotease | | 2017 Sep; 35(9): 864-871. doi: |
| | | 10.1038/nbt.3909. Epub 2017 |
| | | Jun 26. |
| Double STOP Codon | 6 | |
| multiple cloning site | 24 | |
| pLVX-CMV 100 vector | 4039 | Diagonally Scanned Light-Sheet |
| backbone | | Microscopy for Fast Volumetric |
| | | Imaging of Adherent Cells. Dean |
| | | KM, Roudot P, Reis CR, Welf ES, |
| | | Mettlen M, Fiolka R. Biophys J. |
| | | 2016 Mar 29; 110(6): 1456-65. |

The intracellular domain of the Activation CAR contains a transcriptional activator, which, along with the transcriptional acceptor present on the third nucleic acid, serve as a cellular ‘on-switch’ that controls transcription and expression of the therapeutic payload. In some embodiments, the transcriptional activator encodes a Gal4-VP64 fusion protein. The amino acid sequence of a representative Gal4-VP64 fusion protein is set forth below (SEQ ID NO: 86):

```
    1 mkllssiega cdicrlkklk cskekpkcak
        clknnwecry spktkrsplt rahltevesr
  61 lerleqlfll ifpredldmi lkmdslqdik
        alltglfvgd nvnkdavtdr lasvetdmpl
121 tirqhrisat ssseessnkg qrqltvsaaa
        ggsggsggsd alddfdldml gsdalddfdl
181 dmlgsdaldd fdldmlgsda lddfdldmlg
```

In some embodiments, one or more of the domains of the Protease CAR, the Activation CAR, or both the Protease CAR and the Activation CAR are interconnected by a linker. In some embodiments, the Protease CAR and the Activation CAR both have a linker disposed between the transmembrane domain and the intracellular domain. In some embodiments, a linker has an amino acid sequence of GGGX, GGGGX (SEQ ID NO: 87), or GSSGSX (SEQ ID NO: 88), where X is either cysteine (C) or serine(S), or a repeating sequence thereof. In some embodiments, a linker has an amino acid sequence of GGGC (SEQ ID NO: 89), GGGS (SEQ ID NO: 90), GGGGSGGGGSGGGGS (SEQ ID NO: 91), GGGGSGGGGSGGGGSGGGGS (SEQ ID NO: 92), GSTSGSGKPGSGEGSTKG (SEQ ID NO: 93), KESGSVSSEQLAQFRSLD (SEQ ID NO: 94), EGKSSGSGSESKST (SEQ ID NO: 95), or GSAGSAAGSGEF (SEQ ID NO: 96).

In some embodiments, the Activation CAR having the nucleic acid sequence set forth below (SEQ ID NO: 97), and which contains the features set forth in Table 10, and which may be incorporated into a pLVC-CMV 100 construct background:

| | |
|---|---|
| 1 | tggaagggct aattcactcc caaagaagac aagatatcct tgatctgtgg atctaccaca |
| 61 | cacaaggcta cttccctgat tagcagaact acacaccagg gccaggggtc agatatccac |
| 121 | tgacctttgg atggtgctac aagctagtac cagttgagcc agataaggta gaagaggcca |
| 181 | ataaaggaga gaacaccagc ttgttacacc ctgtgagcct gcatgggatg gatgacccgg |
| 241 | agagagaagt gttagagtgg aggtttgaca gccgcctagc atttcatcac gtggcccgag |
| 301 | agctgcatcc ggagtacttc aagaactgct gatatcgagc ttgctacaag ggactttccg |
| 361 | ctggggactt tccagggagg cgtggcctgg gcgggactgg ggagtggcga gccctcagat |
| 421 | cctgcatata agcagctgct ttttgcctgt actgggtctc tctggttaga ccagatctga |
| 481 | gcctgggagc tctctggcta actagggaac ccactgctta agcctcaata aagcttgcct |
| 541 | tgagtgcttc aagtagtgtg tgcccgtctg ttgtgtgact ctggtaacta gagatccctc |
| 601 | agaccctttt agtcagtgtg gaaaatctct agcagtggcg cccgaacagg gacttgaaag |
| 661 | cgaaagggaa accagaggag ctctctcgac gcaggactcg gcttgctgaa gcgcgcacgg |
| 721 | caagaggcga ggggcggcga ctggtgagta cgccaaaaat tttgactagc ggaggctaga |
| 781 | aggagagaga tgggtgcgag agcgtcagta ttaagcgggg gagaattaga tcgcgatggg |
| 841 | aaaaaattcg gttaaggcca gggggaaaga aaaaatataa attaaaacat atagtatggg |
| 901 | caagcaggga gctagaacga ttcgcagtta atcctggcct gttagaaaca tcagaaggct |
| 961 | gtagacaaat actgggacag ctacaaccat cccttcagac aggatcagaa gaacttagat |
| 1021 | cattatataa tacagtagca accctctatt gtgtgcatca aaggatagag ataaaagaca |
| 1081 | ccaaggaagc tttagacaag atagaggaag agcaaaacaa aagtaagacc accgcacagc |
| 1141 | aagcggccgg ccgctgatct tcagacctgg aggaggagat atgagggaca attggagaag |
| 1201 | tgaattatat aaatataaag tagtaaaaat tgaaccatta ggagtagcac ccaccaaggc |
| 1261 | aaagagaaga gtggtgcaga gagaaaaaag agcagtggga ataggagctt tgttccttgg |
| 1321 | gttcttggga gcagcaggaa gcactatggg cgcagcgtca atgacgctga cggtacaggc |
| 1381 | cagacaatta ttgtctggta tagtgcagca gcagaacaat ttgctgaggg ctattgaggc |
| 1441 | gcaacagcat ctgttgcaac tcacagtctg gggcatcaag cagctccagg caagaatcct |
| 1501 | ggctgtggaa agatacctaa aggatcaaca gctcctgggg atttggggtt gctctggaaa |
| 1561 | actcatttgc accactgctg tgccttggaa tgctagttgg agtaataaat ctctggaaca |
| 1621 | gatttggaat cacacgacct ggatggagtg ggacagagaa attaacaatt acacaagctt |
| 1681 | aatacactcc ttaattgaag aatcgcaaaa ccagcaagaa aagaatgaac aagaattatt |
| 1741 | ggaattagat aaatgggcaa gtttgtggaa ttggtttaac ataacaaatt ggctgtggta |
| 1801 | tataaaatta ttcataatga tagtaggagg cttggtaggt ttaagaatag tttttgctgt |
| 1861 | actttctata gtgaatagag ttaggcaggg atattcacca ttatcgtttc agacccacct |
| 1921 | cccaaccccg aggggacccg acaggcccga aggaatagaa gaagaaggtg gagagagaga |
| 1981 | cagagacaga tccattcgat tagtgaacgg atctcgacgg tatcgccttt aaaagaaaag |
| 2041 | gggggattgg ggggtacagt gcaggggaaa gaatagtaga cataatagca acagacatac |
| 2101 | aaactaaaga attacaaaaa caaattacaa aaattcaaaa ttttcgggtt tattacaggg |
| 2161 | acagcagaga tccagtttat cgatgagtaa ttcatacaaa aggactcgcc cctgccttgg |
| 2221 | ggaatcccag ggaccgtcgt taaactccca ctaacgtaga acccagagat cgctgcgttc |
| 2281 | ccgccccctc acccgcccgc tctcgtcatc actgaggtgg agaagagcat gcgtgaggct |
| 2341 | ccggtgcccg tcagtgggca gagcgcacat cgcccacagt ccccgagaag ttggggggag |
| 2401 | gggtcggcaa ttgaaccggt gcctagagaa ggtggcgcgg ggtaaactgg gaaagtgatg |
| 2461 | tcgtgtactg gctccgcctt tttcccgagg gtgggggaga accgtatata agtgcagtag |
| 2521 | tcgccgtgaa cgttcttttt cgcaacgggt ttgccgccag aacacaggta agtgccgtgt |
| 2581 | gtggttcccg cgggcctggc ctctttacgg gttatggccc ttgcgtgcct tgaattactt |
| 2641 | ccacgcccct ggctgcagta cgtgattctt gatcccgagc ttcgggttgg aagtgggtgg |
| 2701 | gagagttcga ggccttgcgc ttaaggagcc ccttcgcctc gtgcttgagt tgaggcctgg |
| 2761 | cctgggcgct ggggccgccg cgtgcgaatc tggtggcacc ttcgcgcctg tctcgctgct |
| 2821 | ttcgataagt ctctagccat ttaaaatttt tgatgacctg ctgcgacgct ttttttctgg |
| 2881 | caagatagtc ttgtaaatgc gggccaagat ctgcacactg gtatttcggt ttttggggcc |
| 2941 | gcgggcggcg acggggcccg tgcgtcccag cgcacatgtt cggcgaggcg gggcctgcga |
| 3001 | gcgcggccac cgagaatcgg acgggggtag tctcaagctg gccggcctgc tctggtgcct |
| 3061 | ggcctcgcgc cgccgtgtat cgccccgccc tgggcggcaa ggctggcccg gtcggcacca |
| 3121 | gttgcgtgag cgggaaagat gccgcttccc ggccctgctg cagggagctc aaaatggagg |
| 3181 | acgcggcgct cgggagagcg ggcgggtgag tcacccacac aaaggaaaag ggcctttccg |
| 3241 | tcctcagccg tcgcttcatg tgactccacg gagtaccggg cgccgtccag gcacctcgat |
| 3301 | tagttctcga gcttttggag tacgtcgtct ttaggttggg gggaggggtt ttatgcgatg |
| 3361 | gagtttcccc acactgagtg ggtggagact gaagttaggc cagcttggca cttgatgtaa |
| 3421 | ttctccttgg aatttgccct ttttgagttt ggatcttggt tcattctcaa gcctcagaca |
| 3481 | gtggttcaaa gtttttttct tccatttcag gtgtcgtgat tcgaattcgc cgccaccatg |
| 3541 | gccttaccag tgaccgcctt gctcctgccg ctggccttgc tgctccacgc cgccaggccg |
| 3601 | gactacaagg acgacgatga caaggatatc cagatgaccc agagcccgtc gagcctttcc |
| 3661 | gcctccgtgg gggacagggt cactatcacc tgctctgctt cccaggacat ctctaactac |
| 3721 | ctgaattggt accagcagaa gcccggcaag gcgcccaagc tactcattta ctacaccagc |
| 3781 | aacctgcact ctggtgtgcc tagccgcttt tcaggttccg gctccggcac cgacttcacc |
| 3841 | ctgactattt cgagtttgca gccagaggac ttcgccacct actattgtca acagtaccgc |
| 3901 | aagctgccgt ggaccttcgg acagggcaca aaactggaga tcaagggtgg cggtggctcg |
| 3961 | ggcggtggtg ggtcgggtgg cggcggatct caagtgcagc tggtgcagag cggggcagaa |
| 4021 | gtcaagaagc ctggctcctc tgtaaaggtg tcatgcaagg cttccggtgg cacgttcagc |
| 4081 | aactattgga tgcattgggt ccgccaggcc cccggacagg gcctggagtg gatgggggcc |
| 4141 | acctaccgtg gccacagcga tacttactac aaccagaaat ttaaaggccg cgtgaccatc |
| 4201 | accgcggaca agtcgacctc cacagcctac atggagctgt ctagtttgcg ctcggaggac |
| 4261 | actgctgttt attactgtgc gcggggtgcc atttacgacg gctacgatgt gctcgacaat |
| 4321 | tggggccagg gaacccttgt caccgtgtcc tctaccacga cgccagcgcc gcgaccacca |
| 4381 | acaccggcgc ccaccatcgc gtcgcagccc ctgtccctgc gcccagaggc gtgccggcca |
| 4441 | gcggcggggg gcgcagtgca cacgaggggg ctggacttcg cctgtgattt ttgggtgctg |
| 4501 | gtggtggttg gtggagtcct ggcttgctat agcttgctag taacagtggc ctttattatt |
| 4561 | ttctgggtga ggagtaagag gagcaggctc ctgcacagtg actacatgaa catgactccc |
| 4621 | cgccgccccg ggcccacccg caagcattac cagccctaty ccccaccacg cgacttcgca |
| 4681 | gcctatcgct ccaaacgggg cagaaagaaa ctcctgtata tattcaaaca accatttatg |
| 4741 | agaccagtac aaactactca agaggaagat ggctgtagct gccgatttcc agaagaagaa |
| 4801 | gaaggaggat gtgaactgag agtgaagttc agcaggagcg cagacgcccc cgcgtaccag |
| 4861 | cagggccaga accagctcta taacgagctc aatctaggac gaagagagga gtacgatgtt |
| 4921 | ttggacaaga gacgtggccg ggaccctgag atggggggaa agccgagaag gaagaaccct |
| 4981 | caggaaggcc tgtacaatga actgcagaaa gataagatgg cggaggccta cagtgagatt |
| 5041 | gggatgaaag gcgagcgccg gaggggcaag gggcacgatg gcctttacca gggtctcagt |
| 5101 | acagccacca aggacaccta cgacgccctt cacatgcagg ccctgccccc tcgcgagaac |
| 5161 | ctgtacttcc agatgatgaa actgctgagc tcgattgagc aggcctgcga catctgtaga |
| 5221 | ctcaagaagc tgaagtgctc taaggagaaa ccaaaatgtg ccaagtgcct gaagaacaac |
| 5281 | tgggaatgtc gctactcccc caagacaaag cgcagccctc tgacccgcgc ccacttgacc |
| 5341 | gaggtggaga gccgtctgga gcgcctggaa cagctgttcc tgctcatctt cccgagggag |
| 5401 | gacctggaca tgatcctgaa aatggattcg ctgcaggaca tcaaggctct tctgaccggc |
| 5461 | ctcttcgtgc aggacaacgt gaacaaggac gccgtcaccg accgcctggc ctctgtggag |
| 5521 | actgacatgc ccctgacgct acggcaacat cgtatttctg ccacctcctc gtccgaggag |
| 5581 | agctcaaata agggccagcg ccagcttact gttagtgctg cggctggcgg gtccggtggg |
| 5641 | tctggcggtt ccgacgctct ggatgacttt gatctggaca tgttgggaag tgatgcgctc |
| 5701 | gatgatttcg acttggacat gcttggctcc gacgcacttg atgacttcga cctcgacatg |
| 5761 | ttgggaagcg acgcgctgga cgattttgac ctggacatgc taggctccgg cgcgccggag |
| 5821 | ggcagaggca gcctgctgac ctgcggcgac gtggaggaga accccggccc catgtggctg |
| 5881 | cagagcctgc tgctcttggg cactgtggcc tgcagcatct ctcgcaaagt gtgtaacgga |
| 5941 | ataggtattg gtgaatttaa agactcactc tccataaatg ctacgaatat taaacacttc |
| 6001 | aaaaactgca cctccatcag tggcgatctc cacatcctgc cggtggcatt taggggtgac |
| 6061 | tccttcacac atactcctcc tctggatcca caggaactgg atattctgaa aaccgtaaag |
| 6121 | gaaatcacag ggtttttgct gattcaggct tggcctgaaa acaggacgga cctccatgcc |
| 6181 | tttgagaacc tagaaatcat acgcggcagg accaagcaac atggtcagtt ttctcttgca |
| 6241 | gtcgtcagcc tgaacataac atccttggga ttacgctccc tcaaggagat aagtgatgga |
| 6301 | gatgtgataa tttcaggaaa caaaaatttg tgctatgcaa atacaataaa ctggaaaaaa |
| 6361 | ctgtttggga cctccggtca gaaaaccaaa attataagca acagaggtga aaacagctgc |
| 6421 | aaggccacag gccaggtctg ccatgccttg tgctcccccg agggctgctg gggcccggag |
| 6481 | cccagggact gcgtctcttg ccggaatgtc agccgaggca gggaatgcgt ggacaagtgc |
| 6541 | aaccttctgg agggtgagcc aagggagttt gtggagaact ctgagtgcat acagtgccac |
| 6601 | ccagagtgcc tgcctcaggc catgaacatc acctgcacag gacggggacc agacaactgt |
| 6661 | atccagtgtg cccactacat tgacggcccc cactgcgtca agacctgccc ggcaggagtc |
| 6721 | atgggagaaa acaacaccct ggtctggaag tacgcagacg ccggccatgt gtgccacctg |
| 6781 | tgccatccaa actgcaccta cggatgcact gggccaggtc ttgaaggctg tccaacgaat |
| 6841 | gggcctaaga tcccgtccat cgccactggg atggtggggg ccctcctctt gctgctggtg |
| 6901 | gtggccctgg ggatcggcct cttcatgtaa taatctagaa cgcgtctgga acaatcaacc |
| 6961 | tctggattac aaaatttgtg aaagattgac tggtattctt aactatgttg ctccttttac |
| 7021 | gctatgtgga tacgctgctt taatgccttt gtatcatgct attgcttccc gtatggcttt |
| 7081 | cattttctcc tccttgtata aatcctggtt gctgtctctt tatgaggagt tgtggcccgt |
| 7141 | tgtcaggcaa cgtggcgtgg tgtgcactgt gtttgctgac gcaaccccca ctggttgggg |
| 7201 | cattgccacc acctgtcagc tcctttccgg gactttcgct ttccccctcc ctattgccac |
| 7261 | ggcggaactc atcgccgcct gccttgcccg ctgctggaca ggggctcggc tgttgggcac |
| 7321 | tgacaattcc gtggtgttgt cggggaagct gacgtccttt ccatggctgc tcgcctgtgt |
| 7381 | tgccacctgg attctgcgcg ggacgtcctt ctgctacgtc ccttcggccc tcaatccagc |
| 7441 | ggaccttcct tcccgcggcc tgctgccggc tctgcggcct cttccgcgtc ttcgccttcg |
| 7501 | ccctcagacg agtcggatct ccctttgggc cgcctccccg cctggaatta attctgcagt |
| 7561 | cgagacctag aaaaacatgg agcaatcaca agtagcaata cagcagctac caatgctgat |
| 7621 | tgtgcctggc tagaagcaca agaggaggag gaggtgggtt ttccagtcac acctcaggta |
| 7681 | cctttaagac caatgactta caaggcagct gtagatctta gccacttttt aaaagaaaag |
| 7741 | aggggactgg aagggctaat tcactcccaa cgaagacaag atatccttga tctgtggatc |
| 7801 | taccacacac aaggctactt ccctgattag cagaactaca caccagggcc aggggtcaga |
| 7861 | tatccactga cctttggatg gtgctacaag ctagtaccag ttgagccaga taaggtagaa |
| 7921 | gaggccaata aaggagagaa caccagcttg ttacaccctg tgagcctgca tgggatggat |
| 7981 | gacccggaga gagaagtgtt agagtggagg tttgacagcc gcctagcatt tcatcacgtg |
| 8041 | gcccgagagc tgcatccgga gtacttcaag aactgctgat atcgagcttg ctacaaggga |
| 8101 | ctttccgctg gggactttcc agggaggcgt ggcctgggcg ggactgggga gtggcgagcc |
| 8161 | ctcagatcct gcatataagc agctgctttt tgcctgtact gggtctctct ggttagacca |
| 8221 | gatctgagcc tgggagctct ctggctaact agggaaccca ctgcttaagc ctcaataaag |
| 8281 | cttgccttga gtgcttcaag tagtgtgtgc ccgtctgttg tgtgactctg gtaactagag |
| 8341 | atccctcaga cccttttagt cagtgtggaa aatctctagc agtagtagtt catgtcatct |
| 8401 | tattattcag tatttataac ttgcaaagaa atgaatatca gagagtgaga ggccttgaca |
| 8461 | ttgctagcgt ttaccgtcga cctctagcta gagcttggcg taatcatggt catagctgtt |
| 8521 | tcctgtgtga aattgttatc cgctcacaat tccacacaac atacgagccg gaagcataaa |
| 8581 | gtgtaaagcc tggggtgcct aatgagtgag ctaactcaca ttaattgcgt tgcgctcact |
| 8641 | gcccgctttc cagtcgggaa acctgtcgtg ccagctgcat taatgaatcg gccaacgcgc |
| 8701 | ggggagaggc ggtttgcgta ttgggcgctc ttccgcttcc tcgctcactg actcgctgcg |
| 8761 | ctcggtcgtt cggctgcggc gagcggtatc agctcactca aaggcggtaa tacggttatc |
| 8821 | cacagaatca ggggataacg caggaaagaa catgtgagca aaaggccagc aaaaggccag |
| 8881 | gaaccgtaaa aaggccgcgt tgctggcgtt tttccatagg ctccgccccc ctgacgagca |
| 8941 | tcacaaaaat cgacgctcaa gtcagaggtg gcgaaacccg acaggactat aaagatacca |
| 9001 | ggcgtttccc cctggaagct ccctcgtgcg ctctcctgtt ccgaccctgc cgcttaccgg |
| 9061 | atacctgtcc gcctttctcc cttcgggaag cgtggcgctt tctcatagct cacgctgtag |
| 9121 | gtatctcagt tcggtgtagg tcgttcgctc caagctgggc tgtgtgcacg aaccccccgt |
| 9181 | tcagcccgac cgctgcgcct tatccggtaa ctatcgtctt gagtccaacc cggtaagaca |
| 9241 | cgacttatcg ccactggcag cagccactgg taacaggatt agcagagcga ggtatgtagg |
| 9301 | cggtgctaca gagttcttga agtggtggcc taactacggc tacactagaa gaacagtatt |
| 9361 | tggtatctgc gctctgctga agccagttac cttcggaaaa agagttggta gctcttgatc |
| 9421 | cggcaaacaa accaccgctg gtagcggtgg tttttttgtt tgcaagcagc agattacgcg |
| 9481 | cagaaaaaaa ggatctcaag aagatccttt gatcttttct acggggtctg acgctcagtg |
| 9541 | gaacgaaaac tcacgttaag ggattttggt catgagatta tcaaaaagga tcttcaccta |
| 9601 | gatcctttta aattaaaaat gaagttttaa atcaatctaa agtatatatg agtaaacttg |
| 9661 | gtctgacagt taccaatgct taatcagtga ggcacctatc tcagcgatct gtctatttcg |
| 9721 | ttcatccata gttgcctgac tccccgtcgt gtagataact acgatacggg agggcttacc |
| 9781 | atctggcccc agtgctgcaa tgataccgcg agacccacgc tcaccggctc cagatttatc |
| 9841 | agcaataaac cagccagccg gaagggccga gcgcagaagt ggtcctgcaa ctttatccgc |
| 9901 | ctccatccag tctattaatt gttgccggga agctagagta agtagttcgc cagttaatag |
| 9961 | tttgcgcaac gttgttgcca ttgctacagg catcgtggtg tcacgctcgt cgtttggtat |
| 10021 | ggcttcattc agctccggtt cccaacgatc aaggcgagtt acatgatccc ccatgttgtg |
| 10081 | caaaaaagcg gttagctcct tcggtcctcc gatcgttgtc agaagtaagt tggccgcagt |
| 10141 | gttatcactc atggttatgg cagcactgca taattctctt actgtcatgc catccgtaag |
| 10201 | atgcttttct gtgactggtg agtactcaac caagtcattc tgagaatagt gtatgcggcg |
| 10261 | accgagttgc tcttgcccgg cgtcaatacg ggataatacc gcgccacata gcagaacttt |
| 10321 | aaaagtgctc atcattggaa aacgttcttc ggggcgaaaa ctctcaagga tcttaccgct |
| 10381 | gttgagatcc agttcgatgt aacccactcg tgcacccaac tgatcttcag catcttttac |
| 10441 | tttcaccagc gtttctgggt gagcaaaaac aggaaggcaa aatgccgcaa aaaagggaat |
| 10501 | aagggcgaca cggaaatgtt gaatactcat actcttcctt tttcaatatt attgaagcat |
| 10561 | ttatcagggt tattgtctca tgagcggata catatttgaa tgtatttaga aaaataaaca |
| 10621 | aataggggtt ccgcgcacat ttccccgaaa agtgccacct gacgtcgacg gatcgggaga |
| 10681 | tcaacttgtt tattgcagct tataatggtt acaaataaag caatagcatc acaaatttca |
| 10741 | caaataaagc atttttttca ctgcattcta gttgtggttt gtccaaactc atcaatgtat |
| 10801 | cttatcatgt ctggatcaac tggataactc aagctaacca aaatcatccc aaacttccca |
| 10861 | ccccataccc tattaccact gccaattacc tgtggtttca tttactctaa acctgtgatt |
| 10921 | cctctgaatt attttcattt taaagaaatt gtatttctta aatatgtact acaaacttag |
| 10981 | tagt |

TABLE 10
Activation CAR nucleic acid construct

| | Length | |
|---|---|---|
| Name | (bp) | Reference |
| pLVX-CMV 100 vector backbone | 2178 | Dean et al., Biophys. J. 110(6): 1456-65 |
| | | (2016) |
| multiple cloning site | 6 | |
| EF-1α | 1335 | Human elongation factor EF-1-alpha gene, |
| | | Sequence ID: J04617.1 |
| multiple cloning site | 12 | |
| Kozak sequence | 6 | Addgene plasmid # 60360 |
| Human CD8A transcript | 63 | Homo sapiens CD8A, transcript variant 1, |
| | | mRN/A |
| | | Sequence ID: NM_001768.6 |
| FLAG Tag (DYKDDDDK (SEQ ID NO: | 24 | Wang W., et., al., Nat Biotechnol. 2017 |
| 98)) | | September; 35(9): 864-871. doi: 10.1038/ |
| | | nbt.3909. Epub 2017 Jun. 26. |
| VL of anti-tumor protein B | 321 | 10797L\|belantamab\|Humanized\|L-KAPPA |
| | | (V-KAPPA (1-107) [D1] from IMGT |
| Linker_(G4S)3 | 45 | Synthetic construct anti-tumor scFv antibody |
| | | gene. Sequence ID: AF363774.1 |
| VH of anti-tumor protein B | 363 | 10797H\|belantamab\|Humanized\|H- |
| | | GAMMA-1 (VH (1-121) [D1] from IMGT |
| Hinge | 135 | Homo sapiens CD8A, transcript variant 1. |
| | | Sequence ID: NM_001768.6. |
| CD28 Transmembrane Intracellular | 204 | Homo sapiens CD28, transcript variant |
| | | 1. Sequence ID: NM_006139.4. |
| 4-1BB_Cytoplasmic domain | 126 | Homo sapiens TNFRSF9. Sequence ID: |
| | | NM_001561.5. |
| CD3 Zeta Intracellular region | 336 | Homo sapiens CD247, transcript variant |
| | | 2. Sequence ID: NM_000734.4. |
| TEVprotease_cleavage_site | 21 | Wang W., et., al., Nat Biotechnol. 2017 |
| (ENLYFQM (SEQ ID NO: 81)) | | September; 35(9): 864-871. doi: 10.1038/ |
| | | nbt.3909. Epub 2017 Jun 26. |
| GAL4-VP64 | 633 | Morsut L., et. al., Cell. 2016 Feb. |
| | | 11; 164(4): 780-91. |
| | | doi: 10.1016/j.cell.2016.01.012. Epub 2016 |
| | | January 28. |
| AscI Restriction Site | 9 | |
| T2A | 54 | T2A without GSG, addition/A1 9 bp instead |
| | | 6bp rest |
| CSF2 SigN/A1 peptide | 51 | Homo sapiens colony stimulating factor 2 |
| | | (CSF2). Sequence ID: NM_000758.4. |
| EGFR Extracellular Transmembrane | 1005 | Homo sapiens epidermal growth factor |
| domain | | receptor (EGFR), transcript variant 1. |
| | | Sequence ID: NM_005228.5. |
| Double STOP Codon | 6 | |
| multiple cloning site | 12 | |
| pLVX-CMV 100 vector backbone | 4039 | Diagonally Scanned Light-Sheet Microscopy |
| | | for Fast Volumetric Imaging of Adherent |
| | | Cells. Dean KM, Roudot P, Reis CR, Welf |
| | | ES, Mettlen M, Fiolka R. Biophys J. 2016 |
| | | Mar. 29; 110(6): 1456-65. |

The third nucleic acid contains a transcriptional acceptor, a third promoter, and a nucleic acid that encodes a leader peptide and a therapeutic payload operatively linked to the third promoter. Once the transcriptional activator is cleaved from the Activation CAR by the protease of the Protease CAR, the transcriptional activator binds to the transcriptional acceptor. Binding of the transcriptional activator to the transcriptional acceptor initiates transcription of nucleic acid encoding the leader peptide and the therapeutic payload.

In some embodiments, the transcriptional acceptor is a Gal4 binding site or a repetition of Gal4 bindings sites. In some embodiments, the transcriptional acceptor has the nucleic acid sequence GGAGCACTGTCCTCCGAACG (SEQ ID NO: 99). In some embodiments, the transcription acceptor contains two or more, e.g., 2-4 repetitions of the sequence.

The third promoter is operatively linked to the nucleic acid encoding the leader peptide and the therapeutic payload. The third promoter and the transcriptional acceptor enable transcription of the therapeutic payload. In some embodiments, the third promoter is a modified CMV promoter.

The therapeutic payload enables CAR-independent tumor cell killing. The therapeutic payload may be soluble, or membrane bound. The term “soluble” as used herein when referring to a therapeutic payload refers to protein that lacks a transmembrane domain, and when expressed from a cell, is not attached or associated with the cell membrane. The nucleic acid encoding the leader peptide and therapeutic payload is transcribed after the transcriptional activator binds the transcriptional acceptor. The leader peptide ensures secretion of the therapeutic payload into the extracellular environment.

In some embodiments, the Protease CAR, the Activation CAR, the therapeutic payload, or each of the Protease CAR, the Activation CAR, and the therapeutic payload further includes a leader peptide. The term “leader peptide” as used herein refers to a short (e.g., 5-30 or 10-100 amino acids long) stretch of amino acids at the N-terminus of a protein or incorporated in the transmembrane domain of a protein that directs the transport of the protein. Leader peptide-containing proteins will be either be trafficked to the plasma membrane or secreted from the cell. Typically, proteins with a leader peptide and no transmembrane domain will be secreted.

In some embodiments, the leader peptide is derived from the albumin, CD8α, CD33, erythropoietin, IL-2, human or mouse Ig-kappa chain V-III (IgK VIII), tissue plasminogen activator (tPA), or secreted alkaline phosphatase (SEAP). Suitable leader peptides are synthetic sequences derivable from native sequences. Amino acid sequences of representative leader peptides are listed in Table 11:

TABLE 11
Amino Acid Sequences of Leader peptides

| Signal peptide | Sequence |
|---|---|
| Albumin (SEQ ID NO: 100) | MKWVTFISLLFLFSSAYS |
| Synthetic, modified albumin (SEQ ID NO: 101) | MKWVTFISLLFLFSSSSRA |
| CD8α (SEQ ID NO: 102) | MALPVTALLLPLALLLHAARP |
| CD33 (SEQ ID NO: 103) | MPLLLLLPLLWAGALA |
| Erythropoietin (EPO) (SEQ ID NO: 104) | MGVHECPAWLWLLLSLLSLPLGLPVLG |
| IL-2 (SEQ ID NO: 105) | MYRMQLLSCIALSLALVINS |
| Mouse IgK VIII (SEQ ID NO: 106) | METDTLLLWVLLLWVPGSTG |
| Human IgK VIII (SEQ ID NO: 107) | MEAPAQLLFLLLLWLPDTTG |
| Human IgK V-IV (SEQ ID NO: 108) | MVLQTQVFISLLLWISGAYG |
| Synthetic, modified human IgK VIII (SEQ ID NO: | MEAPAQLLFLLLLWLPSSRA |
| 109) | |
| tPA (SEQ ID NO: 110) | MDAMKRGLCCVLLLCGAVFVSPS |
| SEAP (SEQ ID NO: 111) | MLLLLLLLGLRLQLSLG |
| Consensus (SEQ ID NO: 112) | MLLLLLLLLLLALALA |
| Synthetic secrecon (SEQ ID NO: 113) | MWWRLWWLLLLLLLLWPMVWA |

In some embodiments, the therapeutic payload is a soluble antibody fragment, a cytokine, a soluble cytokine receptor, a chemokine, a soluble chemokine receptor, or an oligopeptide or RNA vaccine.

In some embodiments, the therapeutic payload is an antibody fragment that binds CD3, CD19, or CD20. The fragments are derivable from intact antibodies that bind CD3, CD19 and CD20. For example, representative examples of antibodies that bind CD3 include blinatumomab (Blincyto®), catumaxomab (Removab®), flotetuzumab (MGD006), muromonab-CD3 (Orthoclone OKT3®), otelixizumab (ChAglyCD3, TRX4), teplizumab, and visilizumab.

Representative antibodies that bind CD19 include loncastuximab (Zynlonta®), tafasitamab (Monjuvi®), denintuzumab (SGN-CD19A), and inebilizumab (Uplizna®).

Representative antibodies that bind CD20 include ofatumamab (Kesimpta®), obinutuzumab (Gazyva®), ocaratuzumab, ublituximab, veltuzumab (IMMU-106), tositumomab (Bexxar®), and rituximab (Rituxan®).

In some embodiments, the therapeutic payload is an antibody fragment that binds to a tolerogenic molecule or a checkpoint inhibitor, representative examples of which include HLA-E, TGFβ, CTLA-4, PD1, PD-L1, PD-L2, TIGIT, TIM3, LAG3, EGFR, and NKG2A.

Representative antibodies that bind HLA-E or its ligand NKG2A are known in the art. See, e.g., U.S. Pat. Nos. 8,206,709, 10,676,523, 10,870,700, and 11,225,519 and U.S. Patent Application Publication 2012/0171195. In some embodiments, the therapeutic payload is an antibody fragment derived from a commercially available anti-NKG2A antibody, antibody fragment, or variant thereof, e.g., monalizumab (IPH2201) and humanized Z199. Amino acid sequences of representative anti-NKG2A heavy and light chains are set forth Table 12:

TABLE 12
Amino Acid Sequences of anti-NKG2A antibody fragments

| Polypeptide | Sequence | |
|---|---|---|
| monalizumab | 1 | evqlvqsgae vkkpgeslki sckgsgysft sywmnwvrqm pgkglewmgr idpydsethy |
| heavy chain | 61 | spsfqgqvti sadksistay lqwsslkasd tamyycargg ydfdvgtlyw ffdvwgqgtt |
| (SEQ ID NO: | 121 | vtvssastkg psvfplapcs rstsestaal gclvkdyfpe pvtvswnsga ltsgvhtfpa |
| 114) | 181 | vlqssglysl ssvvtvpsss lgtktytcnv dhkpsntkvd krveskygpp cppcpapefl |
| | 241 | ggpsvflfpp kpkdtlmisr tpevtcvvvd vsqedpevqf nwyvdgvevh naktkpreeq |
| | 301 | fnstyrvvsv ltvlhqdwln gkeykckvsn kglpssiekt iskakgqpre pqvytlppsq |
| | 361 | eemtknqvsl tclvkgfyps diavewesng qpennykttp pvldsdgsff lysrltvdks |
| | 421 | rwqegnvfsc svmhealhnh ytqkslslsl gk |
| monalizumab | 1 | diqmtqspss lsasvgdrvt itcraseniy sylawyqqkp gkapklliyn aktlaegvps |
| light chain (SEQ | 61 | rfsgsgsgtd ftltisslqp edfatyycqh hygtprtfgg gtkveikrtv aapsvfifpp |
| ID NO: 115) | 121 | sdeqlksgta svvcllnnfy preakvqwkv dnalqsgnsq esvteqdskd styslsstlt |
| | 181 | lskadyekhk vyacevthqg lsspvtksfn rgec |
| humZ199 heavy | 1 | evqlvesggg lvkpggslrl scaasgftfs syamswvrqa pgkglewvse issggsytyy |
| chain (SEQ ID | 61 | adsvkgrfti srdnaknsly lqmnslraed tavyycarhg dyprffdvwg qgttvtvss |
| NO: 116) | | |
| hum Z199 light | 1 | eivltqspat lslspgerat lscsasssvs syiywyqqkp gqaprlliyl tsnlasgipa |
| chain (SEQ ID | 61 | rfsgsgsgtd ftltisslep edfavyycqq wsgnpytfgq gtkleik |
| NO: 117) | | |

Representative antibodies that bind TGFβ or a receptor thereof are known in the art, e.g., fresolimumab, and U.S. Pat. Nos. 8,147,834, 9,109,031, 9,783,604, and 11,312,767.

Representative antibodies that bind CTLA-4 include bavunalimab (XmAb 22841), botensilimab (AGEN 1181), cadonilimab, ipilimumab (YERVOY®), quavonlimab (MK 1308), tremelimumab (CP-675,206), vudalimab (XmAb 20717 or XmAb 717), and zalifrelimab (AGEN 1884).

Representative antibodies that bind PD1 include balstilimab, budigalimab, cadonilimab, cemiplimab, cetrelimab, dostarlimab, izuralimab, nivolumab, pacmilimab, pembrolizumab, penpulimab, peresolimab, pidilizumab, retifanlimab, rosnilimab, sintilimab, spartalizumab, tislelizumab, toripalimab, volrustomig, vudalimab, zeluvalimab, and zimberelimab. Representative antibodies that bind PD-L1 include atezolizumab, avelumab, bintrafusp alfa, cosibelimab, danburstotug, durvalumab, inbakicept, lodapolimab, pimivalimab, and socazolimab.

Representative antibodies that bind TIGIT are known in the art, e.g., belrestotug, domvanalimab, etigilimab, ociperlimab, tiragolumab, vibostolimab, U.S. Pat. Nos. 10,017,572, 10,766,957, 10,213,505, 10,329,349, and 11,021,537 and U.S. Patent Application Publications 2009/0258013, 2020/0040082, 2020/0354453, and 2021/0087268.

Representative antibodies that bind TIM3 are known in the art, e.g., cobolimab, sabatolimab, surzebiclimab, U.S. Pat. Nos. 10,533,052, and 10,927,171 and U.S. Patent Application Publications 2019/0382480, 2021/0221885, 2021/0261663, 2021/0363242, 2022/0089720, and 2022/0235130.

Representative antibodies that bind LAG3 are known in the art, e.g., bavunalimab (XmAb 22841), ieramilimab, relatlimab, U.S. Pat. Nos. 10,358,495, 10,898,571, 11,028,169, and 11,045,547 and U.S. Patent Application Publications 2019/0330336, 2021/0363243, and 2022/0002410.

Representative antibodies that bind EGFR include cetuximab (Erbitux®), panitumumab (Vectibix®), necitumumab (Portrazza®), and amivantamab (Rybrevant®)

In some embodiments, the therapeutic payload is bispecific and includes two antibody fragments, each binding a different target on a cancer cell. In some embodiments, the therapeutic payload is a bispecific T cell engager containing an antibody fragment that binds an TAA on a cancer cell and an antibody fragment that binds an antigen on a T cell (e.g., CD3). In some embodiments, one antibody fragment binds CD3 and the other binds BCMA, CD19, CD20, CD33, CD38, CD138, EGFR, FCRH5, Flt3, GPCR5D, PSMA, or SLAMF7. Representative antibody sequences provided elsewhere herein may be used to bispecific antibodies and bispecific T cell engagers.

In some embodiments, the therapeutic payload incudes a scFv that binds CD19 and a scFv that binds CD3. Anti-CD19 and anti-CD3 bispecific antibody fragments are known in the art, e.g., blinatumomab (Blincyto®), duvortuxizumab, U.S. Pat. Nos. 7,112,324, 8,840,888, 10,191,034, 10,633,443, and 10,889,653 and U.S. Patent Application Publications 2016/0355588 and 2021/0317212. The amino acid sequence of a representative bispecific antibody fragment that binds CD3 and CD19 is set forth below (SEQ ID NO: 118):

| | |
|---|---|
| 1 | diqltqspas lavslgqrat isckasqsvd ydgdsylnwy qqipgqppkl liydasnlvs |
| 61 | gipprisgsg sgtdftlnih pvekvdaaty hcqqstedpw tfgggtklei kggggsgggg |
| 121 | sggggsqvql qqsgaelvrp gssvkiscka sgyafssywm nwvkqrpgqg lewiggiwpg |
| 181 | dgdtnyngkf kgkatltade ssstaymqls slasedsavy fcarretttv gryyyamdyw |
| 241 | gqgttvtvss ggggsdiklq qsgaelarpg asvkmsckts gytftrytmh wvkqrpgqgl |
| 301 | ewigyinpsr gytnynqkfk dkatlttdks sstaymqlss ltsedsavyy caryyddhyc |
| 361 | ldywgqgttl tvssveggsg gsggsggsgg vddiqltqsp aimsaspgek vtmtcrasss |
| 421 | vsymnwyqqk sgtspkrwiy dtskvasgvp yrfsgsgsgt sysltissme aedaatyycq |
| 481 | qwssnpltfg agtklelk |

In some embodiments, the therapeutic payload incudes a scFv that binds FCRH5 and a scFv that binds CD3. The amino acid sequence of a representative bispecific antibody fragment that binds CD3 and FCRH5 is set forth below (SEQ ID NO: 119):

| | |
|---|---|
| 1 | diqmtqspss lsasvgdrvt itckasqdvr nlvvwfqqkp gkapklliys gsyrysgvps |
| 61 | rfsgsgsgtd ftltissiqp edfatyycqq hysppytfgq gtkveikggg gsggggsggg |
| 121 | gsevqlvesg pglvkpsetl sltctvsgfs ltrfgvhwvr qppgkglewl gviwrggstd |
| 181 | ynaafvsrlt iskdnsknqv slklssvtaa dtavyycsnh yygssdyald nwgqgtlvtv |
| 241 | ssggggsevq lvqsgaevkk pgasvkvsck asgftftsyy ihwvrqapgq glewigwiyp |
| 301 | endntkynek fkdrvtitad tststaylel sslrsedtav yycardgysr yyfdywgqgt |
| 361 | lvtvssgggg sggggsgggg sdivmtqspd slavslgera tinckssqsl lnsrtrknyl |
| 421 | awyqqkpgqs pklliywtst rksgvpdrfs gsgsgtdftl tisslqaedv avyyckqsfi |
| 481 | lrtfgggtkv eik |

In some embodiments, the therapeutic payload incudes a scFv that binds CD20 and a scFv that binds CD3. Anti-CD20 and anti-CD3 bispecific antibody fragments are known in the art, e.g., epcoritamab, glofitamab, mosunetuzumab (Lunsumio®), odronextamab, plamotamab, and U.S. Pat. Nos. 10,550,193, 10,662,244, 10,787,520, and 11,440,972.

In some embodiments, the therapeutic payload is an antibody fragment that binds a cytokine or a chemokine. In some embodiments, the therapeutic payload is an antibody fragment binds IL-6 or IL-6R. Such fragments are obtainable from intact anti-IL-6 antibodies, e.g., siltuximab (Sylvant®), sirukumab, and U.S. Pat. Nos. 8,062,866, 8,309,300, and 9,834,603, and anti-IL-6R antibodies, e.g., sarilumab (Kevzara®), satralizumab (Enspryng®), tocilizumab (Actemra®), U.S. Pat. Nos. 8,753,634, 9,884,916, and 10,081,628 and U.S. Application Publications 2012/0045440, 2013/0317203, and 2021/0301027.

In some embodiments, the therapeutic payload is a cytokine or a chemokine. In some embodiments, the cytokine or chemokine is IFNγ, soluble IFNγR, TGFβ, IL-1, IL-2, soluble IL-2R, IL-3, IL-4, IL-5, IL-6, IL-7, IL-8, IL-9, or IL-10, amino acid sequences of which are known in the art. The amino acid sequence of a representative IFNγ is set forth below (SEQ ID NO: 120):

| | |
|---|---|
| 1 | mkytsyilaf qlcivlgslg cycqdpyvke aenlkkyfna ghsdvadngt lflgilknwk |
| 61 | eesdrkimqs qivsfyfklf knfkddqsiq ksvetikedm nvkffnsnkk krddfekltn |
| 121 | ysvtdlnvqr kaiheliqvm aelspaaktg krkrsqmlfr grrasq |

The amino acid sequence of a representative IL-2 is set forth below (SEQ ID NO: 121):

| | |
|---|---|
| 1 | myrmqllsci alslalvtns aptssstkkt qlqlehllld lqmilnginn yknpkltrml |
| 61 | tfkfympkka telkhlqcle eelkpleevl nlaqsknfhl rprdlisnin vivlelkgse |
| 121 | ttfmceyade tativeflnr witfcqsiis tlt |

In some embodiments, the therapeutic payload is an RNA or oligopeptide vaccine. Oligopeptide vaccines are short peptides cable of being presented by HLA proteins to cytotoxic T cells and induce cytotoxicity in those cells when they recognize cancers presenting protein from which the oligopeptide vaccine derives. In some embodiments, the therapeutic payload is an RNA or oligopeptide vaccine derived from Survivin, Wilms tumor 1 transcription factor (WT1), mucin 1 (MUC1), melanoma-associated antigen 3 (MAGE-A3), or melanoma-associated antigen C1 (MAGE-C1, also known as CT7). Representative amino acid sequences are provided for WT1 at NCBI Accession No. NP_000369.4, MUC1 at NCBI Accession No. NP_001018016.1, MAGE-A3 at NCBI Accession No. NP_005353.1, and MAGE-C1 at NCBI Accession No. NP 005453.2.

In some embodiments, the therapeutic payload is localized to the plasma membrane of the immune cell (i.e., membrane-bound). Membrane-bound therapeutic payloads include cell surface receptors (e.g., cytokine receptors, chemokine receptors, receptors for inhibitory molecules, CARs), membrane-bound cytokines, membrane-bound chemokines, and membrane-bound antibodies.

In some embodiments, the therapeutic payload is a surface receptor. In some embodiments, the surface receptor is a CAR containing a combination or subcombination of the domains described herein. In some embodiments, the surface receptor is CTLA-4, PD1, PD-L1, PD-L2. In some embodiments, the surface receptor is a receptor for a cytokine or chemokine. Representative cytokine or chemokine receptors include IFNγR, IL-1R, IL-2R, IL-3R, IL-4R, IL-5R, IL-6R, IL-7R, IL-8R, IL-9R, IL-10R, IL-15R, and TGFβR, amino acid sequences of which are known in the art. The IL-2R, for example, is a heterocomplex consisting of subunits IL-2Rα (CD25), IL-2Rβ (CD122) and the common-γ chain receptor (CD132). The amino sequence of a representative IL-2Rα (CD28) is set forth below (SEQ ID NO: 122):

| | |
|---|---|
| 1 | mdsyllmwgl ltfimvpgcq aelcdddppe iphatfkama ykegtmlnce ckrgfrriks |
| 61 | gslymlctgn sshsswdnqc gctssatrnt tkqvtpqpee qkerkttemq spmqpvdqas |
| 121 | lpghcreppp weneateriy hfvvgqmvyy qcvqgyralh rgpaesvckm thgktrwtqp |
| 181 | qlictgemet sqfpgeekpq aspegrpese tsclvtttdf qiqtemaatm etsiftteyq |
| 241 | vavagcvfll isvlllsglt wqrrqrksrr ti |

The amino sequence of a representative IL-2Rβ (CD122) is set forth below (SEQ ID NO: 123):

| | |
|---|---|
| 1 | maapalswrl pllilllpla tswasaavng tsqftcfyns raniscvwsq dgalqdtscq |
| 61 | vhawpdrrrw ngtcellpvs qaswacnlil gapdsqkltt vdivtlrvlc regvrwrvma |
| 121 | iqdfkpfenl rlmapislqv vhvethrcni sweisqashy ferhlefear tlspghtwee |
| 181 | aplltlkqkq ewicletltp dtqyefqvrv kplqgefttw spwsqplafr tkpaalgkdt |
| 241 | ipwlghllvg lsgafgfiil vyllincrnt gpwlkkvlkc ntpdpskffs qlssehggdv |
| 301 | qkwlsspfps ssfspgglap eisplevler dkvtqlllqq dkvpepasls snhsltscft |
| 361 | nqgyfffhlp daleieacqv yftydpysee dpdegvagap tgsspqplqp lsgeddayct |
| 421 | fpsrddlllf spsllggpsp pstapggsga geermppslq ervprdwdpq plgpptpgvp |
| 481 | dlvdfqpppe lvlreageev pdagpregvs fpwsrppgqg efralnarlp lntdaylslq |
| 541 | elqgqdpthl v |

The amino sequence of a representative common-γ chain receptor (CD132) is set forth below (SEQ ID NO: 124):

| | |
|---|---|
| 1 | mlkpsipfts llflqlpllg vglnttiltp ngnedttadf flttmptdsl svstlplpev |
| 61 | qcfvfnveym nctwnsssep qptnltlhyw yknsdndkvq kcshylfsee itsgcqlqkk |
| 121 | eihlyqtfvv qlqdprepri qatqmlklqn lvipwapenl tlhklsesql elnwnnrfln |
| 181 | hclehlvqyr tdwdhswteq svdyrhkfsl psvdgqkryt frvrsrfnpl cgsaqhwsew |
| 241 | shpihwgsnt skenpflfal eavvisvgsm gliisllcvy fwlertmpri ptlknledlv |
| 301 | teyhgnisaw sgvskglaes lqpdyserlc lvseippkgg algegpgasp cnqhspywap |
| 361 | pcytlkpet |

The IL-7R is a heterodimer consisting of subunits IL-7Rα (CD127) and the common-γ chain receptor (CD132). The amino sequence of a representative IL-7Rα is set forth below (SEQ ID NO: 125):

| | |
|---|---|
| 1 | mtilgttfgm vfsllqvvsg esgyaqngdl edaelddysf scysqlevng sqhsltcafe |
| 61 | dpdvnitnle feicgalvev kclnfrklqe iyfietkkfl ligksnicvk vgeksltckk |
| 121 | idlttivkpe apfdlsvvyr egandfvvtf ntshlqkkyv kvlmhdvayr qekdenkwth |
| 181 | vnlsstkltl lqrklqpaam yeikvrsipd hyfkgfwsew spsyyfrtpe innssgemdp |
| 241 | illtisilsf fsvallvila cvlwkkrikp ivwpslpdhk ktlehlckkp rknlnvsfnp |
| 301 | esfldcqihr vddiqardev egflqdtfpq qleesekqrl ggdvqspncp sedvvitpes |
| 361 | fgrdssltcl agnvsacdap ilsssrsldc resgkngphv yqdlllslgt tnstlpppfs |
| 421 | lqsgiltlnp vaqgqpilts lgsngeeayv tmssfyqnq |

The IL-15R is a heterodimer consisting of subunits IL-15Ra (CD215), IL-2Rβ (CD122) and the common-γ chain receptor (CD132). The amino sequence of a representative IL-15Rα (CD215) is set forth below (SEQ ID NO: 126):

| | |
|---|---|
| 1 | maprrargcr tlglpallll lllrppatrg itcpppmsve hadiwvksys lysreryicn |
| 61 | sgfkrkagts sltecvlnka tnvahwttps lkcirdpalv hqrpappstv ttagvtpqpe |
| 121 | slspsgkepa asspssnnta attaaivpgs qlmpskspst gtteisshes shgtpsqtta |
| 181 | knweltasas hqppgvypqg hsdttvaist stvllcglsa vsllacylks rqtpplasve |
| 241 | meamealpvt wgtssrdedl encshhl |

In some embodiments, the therapeutic payload is a membrane-bound cytokine or chemokine, amino acid sequences of which are known in the art. In some embodiments, the therapeutic payload is a membrane-bound antibody, amino acid sequences of which are known in the art.

In some embodiments, the third nucleic acid having the nucleic acid sequence set forth below (SEQ ID NO: 127), and which contains the features set forth in Table 13, and which may be incorporated into a pLVC-CMV 100 construct background:

| | |
|---|---|
| 1 | tggaagggct aattcactcc caaagaagac aagatatcct tgatctgtgg atctaccaca |
| 61 | cacaaggcta cttccctgat tagcagaact acacaccagg gccaggggtc agatatccac |
| 121 | tgacctttgg atggtgctac aagctagtac cagttgagcc agataaggta gaagaggcca |
| 181 | ataaaggaga gaacaccagc ttgttacacc ctgtgagcct gcatgggatg gatgacccgg |
| 241 | agagagaagt gttagagtgg aggtttgaca gccgcctagc atttcatcac gtggcccgag |
| 301 | agctgcatcc ggagtacttc aagaactgct gatatcgagc ttgctacaag ggactttccg |
| 361 | ctggggactt tccagggagg cgtggcctgg gcgggactgg ggagtggcga gccctcagat |
| 421 | cctgcatata agcagctgct ttttgcctgt actgggtctc tctggttaga ccagatctga |
| 481 | gcctgggagc tctctggcta actagggaac ccactgctta agcctcaata aagcttgcct |
| 541 | tgagtgcttc aagtagtgtg tgcccgtctg ttgtgtgact ctggtaacta gagatccctc |
| 601 | agaccctttt agtcagtgtg gaaaatctct agcagtggcg cccgaacagg gacttgaaag |
| 661 | cgaaagggaa accagaggag ctctctcgac gcaggactcg gcttgctgaa gcgcgcacgg |
| 721 | caagaggcga ggggcggcga ctggtgagta cgccaaaaat tttgactagc ggaggctaga |
| 781 | aggagagaga tgggtgcgag agcgtcagta ttaagcgggg gagaattaga tcgcgatggg |
| 841 | aaaaaattcg gttaaggcca gggggaaaga aaaaatataa attaaaacat atagtatggg |
| 901 | caagcaggga gctagaacga ttcgcagtta atcctggcct gttagaaaca tcagaaggct |
| 961 | gtagacaaat actgggacag ctacaaccat cccttcagac aggatcagaa gaacttagat |
| 1021 | cattatataa tacagtagca accctctatt gtgtgcatca aaggatagag ataaaagaca |
| 1081 | ccaaggaagc tttagacaag atagaggaag agcaaaacaa aagtaagacc accgcacagc |
| 1141 | aagcggccgg ccgctgatct tcagacctgg aggaggagat atgagggaca attggagaag |
| 1201 | tgaattatat aaatataaag tagtaaaaat tgaaccatta ggagtagcac ccaccaaggc |
| 1261 | aaagagaaga gtggtgcaga gagaaaaaag agcagtggga ataggagctt tgttccttgg |
| 1321 | gttcttggga gcagcaggaa gcactatggg cgcagcgtca atgacgctga cggtacaggc |
| 1381 | cagacaatta ttgtctggta tagtgcagca gcagaacaat ttgctgaggg ctattgaggc |
| 1441 | gcaacagcat ctgttgcaac tcacagtctg gggcatcaag cagctccagg caagaatcct |
| 1501 | ggctgtggaa agatacctaa aggatcaaca gctcctgggg atttggggtt gctctggaaa |
| 1561 | actcatttgc accactgctg tgccttggaa tgctagttgg agtaataaat ctctggaaca |
| 1621 | gatttggaat cacacgacct ggatggagtg ggacagagaa attaacaatt acacaagctt |
| 1681 | aatacactcc ttaattgaag aatcgcaaaa ccagcaagaa aagaatgaac aagaattatt |
| 1741 | ggaattagat aaatgggcaa gtttgtggaa ttggtttaac ataacaaatt ggctgtggta |
| 1801 | tataaaatta ttcataatga tagtaggagg cttggtaggt ttaagaatag tttttgctgt |
| 1861 | actttctata gtgaatagag ttaggcaggg atattcacca ttatcgtttc agacccacct |
| 1921 | cccaaccccg aggggacccg acaggcccga aggaatagaa gaagaaggtg gagagagaga |
| 1981 | cagagacaga tccattcgat tagtgaacgg atctcgacgg tatcgccttt aaaagaaaag |
| 2041 | gggggattgg ggggtacagt gcaggggaaa gaatagtaga cataatagca acagacatac |
| 2101 | aaactaaaga attacaaaaa caaattacaa aaattcaaaa ttttcgggtt tattacaggg |
| 2161 | acagcagaga tccagtttat cgataagctt gatatcgaat tcggagcact gtcctccgaa |
| 2221 | cgtcggagca ctgtcctccg aacgtcggag cactgtcctc cgaacgtcgg agcactgtcc |
| 2281 | tccgaacgga gcatgtcctc cgaacgtcgg agcactgtcc tccgaacgac tagttaggcg |
| 2341 | tgtacggtgg gaggcctata taagcagagc tcgtttagtg aaccgtcaga tcgcctggag |
| 2401 | acgccatcca cgctgttttg acctccatag aagacaccga ctctagagga tccaccggtc |
| 2461 | gccaccatgg tgagcaaggg cgaggagctg ttcaccgggg tggtgcccat cctggtcgag |
| 2521 | ctggacggcg acgtaaacgg ccacaagttc agcgtgtccg gcgagggcga gggcgatgcc |
| 2581 | acctacggca agctgaccct gaagttcatc tgcaccaccg gcaagctgcc cgtgccctgg |
| 2641 | cccaccctcg tgaccaccct gacctacggc gtgcagtgct tcagccgcta ccccgaccac |
| 2701 | atgaagcagc acgacttctt caagtccgcc atgcccgaag gctacgtcca ggagcgcacc |
| 2761 | atcttcttca aggacgacgg caactacaag acccgcgccg aggtgaagtt cgagggcgac |
| 2821 | accctggtga accgcatcga gctgaagggc atcgacttca aggaggacgg caacatcctg |
| 2881 | gggcacaagc tggagtacaa ctacaacagc cacaacgtct atatcatggc cgacaagcag |
| 2941 | aagaacggca tcaaggtgaa cttcaagatc cgccacaaca tcgaggacgg cagcgtgcag |
| 3001 | ctcgccgacc actaccagca gaacaccccc atcggcgacg gccccgtgct gctgcccgac |
| 3061 | aaccactacc tgagcaccca gtccgccctg agcaaagacc ccaacgagaa gcgcgatcac |
| 3121 | atggtcctgc tggagttcgt gaccgccgcc gggatcactc tcggcatgga cgagctgtac |
| 3181 | aagtaagctc gagcagcgct gcgatcgcgt taacgggtag gggaggcgct tttcccaagg |
| 3241 | cagtctggag catgcgcttt agcagccccg ctgggcactt ggcgctacac aagtggcctc |
| 3301 | tggcctcgca cacattccac atccaccggt aggcgccaac cggctccgtt ctttggtggc |
| 3361 | cccttcgcgc caccttctac tcctccccta gtcaggaagt tcccccccgc cccgcagctc |
| 3421 | gcgtcgtgca ggacgtgaca aatggaagta gcacgtctca ctagtctcgt gcagatggac |
| 3481 | agcaccgctg agcaatggaa gcgggtaggc ctttggggca gcggccaata gcagctttgc |
| 3541 | tccttcgctt tctgggctca gaggctggga aggggtgggt ccgggggcgg gctcaggggc |
| 3601 | gggctcaggg gcggggcggg cgcccgaagg tcctccggag gcccggcatt ctgcacgctt |
| 3661 | caaaagcgca cgtctgccgc gctgttctcc tcttcctcat ctccgggcct ttcgacctgc |
| 3721 | agcccaagct taccatggtg agcaagggcg aggaggataa catggccatc atcaaggagt |
| 3781 | tcatgcgctt caaggtgcac atggagggct ccgtgaacgg ccacgagttc gagatcgagg |
| 3841 | gcgagggcga gggccgcccc tacgagggca cccagaccgc caagctgaag gtgaccaagg |
| 3901 | gtggccccct gcccttcgcc tgggacatcc tgtcccctca gttcatgtac ggctccaagg |
| 3961 | cctacgtgaa gcaccccgcc gacatccccg actacttgaa gctgtccttc cccgagggct |
| 4021 | tcaagtggga gcgcgtgatg aacttcgagg acggcggcgt ggtgaccgtg acccaggact |
| 4081 | cctccctgca ggacggcgag ttcatctaca aggtgaagct gcgcggcacc aacttcccct |
| 4141 | ccgacggccc cgtaatgcag aagaagacca tgggctggga ggcctcctcc gagcggatgt |
| 4201 | accccgagga cggcgccctg aagggcgaga tcaagcagag gctgaagctg aaggacggcg |
| 4261 | gccactacga cgctgaggtc aagaccacct acaaggccaa gaagcccgtg cagctgcccg |
| 4321 | gcgcctacaa cgtcaacatc aagttggaca tcacctccca caacgaggac tacaccatcg |
| 4381 | tggaacagta cgaacgcgcc gagggccgcc actccaccgg cggcatggac gagctgtaca |
| 4441 | agtaacatat gcctaggtct agaacgcgtc tggaacaatc aacctctgga ttacaaaatt |
| 4501 | tgtgaaagat tgactggtat tcttaactat gttgctcctt ttacgctatg tggatacgct |
| 4561 | gctttaatgc ctttgtatca tgctattgct tcccgtatgg ctttcatttt ctcctccttg |
| 4621 | tataaatcct ggttgctgtc tctttatgag gagttgtggc ccgttgtcag gcaacgtggc |
| 4681 | gtggtgtgca ctgtgtttgc tgacgcaacc cccactggtt ggggcattgc caccacctgt |
| 4741 | cagctccttt ccgggacttt cgctttcccc ctccctattg ccacggcgga actcatcgcc |
| 4801 | gcctgccttg cccgctgctg gacaggggct cggctgttgg gcactgacaa ttccgtggtg |
| 4861 | ttgtcgggga agctgacgtc ctttccatgg ctgctcgcct gtgttgccac ctggattctg |
| 4921 | cgcgggacgt ccttctgcta cgtcccttcg gccctcaatc cagcggacct tccttcccgc |
| 4981 | ggcctgctgc cggctctgcg gcctcttccg cgtcttcgcc ttcgccctca gacgagtcgg |
| 5041 | atctcccttt gggccgcctc cccgcctgga attaattctg cagtcgagac ctagaaaaac |
| 5101 | atggagcaat cacaagtagc aatacagcag ctaccaatgc tgattgtgcc tggctagaag |
| 5161 | cacaagagga ggaggaggtg ggttttccag tcacacctca ggtaccttta agaccaatga |
| 5221 | cttacaaggc agctgtagat cttagccact ttttaaaaga aaagagggga ctggaagggc |
| 5281 | taattcactc ccaacgaaga caagatatcc ttgatctgtg gatctaccac acacaaggct |
| 5341 | acttccctga ttagcagaac tacacaccag ggccaggggt cagatatcca ctgacctttg |
| 5401 | gatggtgcta caagctagta ccagttgagc cagataaggt agaagaggcc aataaaggag |
| 5461 | agaacaccag cttgttacac cctgtgagcc tgcatgggat ggatgacccg gagagagaag |
| 5521 | tgttagagtg gaggtttgac agccgcctag catttcatca cgtggcccga gagctgcatc |
| 5581 | cggagtactt caagaactgc tgatatcgag cttgctacaa gggactttcc gctggggact |
| 5641 | ttccagggag gcgtggcctg ggcgggactg gggagtggcg agccctcaga tcctgcatat |
| 5701 | aagcagctgc tttttgcctg tactgggtct ctctggttag accagatctg agcctgggag |
| 5761 | ctctctggct aactagggaa cccactgctt aagcctcaat aaagcttgcc ttgagtgctt |
| 5821 | caagtagtgt gtgcccgtct gttgtgtgac tctggtaact agagatccct cagacccttt |
| 5881 | tagtcagtgt ggaaaatctc tagcagtagt agttcatgtc atcttattat tcagtattta |
| 5941 | taacttgcaa agaaatgaat atcagagagt gagaggcctt gacattgcta gcgtttaccg |
| 6001 | tcgacctcta gctagagctt ggcgtaatca tggtcatagc tgtttcctgt gtgaaattgt |
| 6061 | tatccgctca caattccaca caacatacga gccggaagca taaagtgtaa agcctggggt |
| 6121 | gcctaatgag tgagctaact cacattaatt gcgttgcgct cactgcccgc tttccagtcg |
| 6181 | ggaaacctgt cgtgccagct gcattaatga atcggccaac gcgcggggag aggcggtttg |
| 6241 | cgtattgggc gctcttccgc ttcctcgctc actgactcgc tgcgctcggt cgttcggctg |
| 6301 | cggcgagcgg tatcagctca ctcaaaggcg gtaatacggt tatccacaga atcaggggat |
| 6361 | aacgcaggaa agaacatgtg agcaaaaggc cagcaaaagg ccaggaaccg taaaaaggcc |
| 6421 | gcgttgctgg cgtttttcca taggctccgc ccccctgacg agcatcacaa aaatcgacgc |
| 6481 | tcaagtcaga ggtggcgaaa cccgacagga ctataaagat accaggcgtt tccccctgga |
| 6541 | agctccctcg tgcgctctcc tgttccgacc ctgccgctta ccggatacct gtccgccttt |
| 6601 | ctcccttcgg gaagcgtggc gctttctcat agctcacgct gtaggtatct cagttcggtg |
| 6661 | taggtcgttc gctccaagct gggctgtgtg cacgaacccc ccgttcagcc cgaccgctgc |
| 6721 | gccttatccg gtaactatcg tcttgagtcc aacccggtaa gacacgactt atcgccactg |
| 6781 | gcagcagcca ctggtaacag gattagcaga gcgaggtatg taggcggtgc tacagagttc |
| 6841 | ttgaagtggt ggcctaacta cggctacact agaagaacag tatttggtat ctgcgctctg |
| 6901 | ctgaagccag ttaccttcgg aaaaagagtt ggtagctctt gatccggcaa acaaaccacc |
| 6961 | gctggtagcg gtggtttttt tgtttgcaag cagcagatta cgcgcagaaa aaaaggatct |
| 7021 | caagaagatc ctttgatctt ttctacgggg tctgacgctc agtggaacga aaactcacgt |
| 7081 | taagggattt tggtcatgag attatcaaaa aggatcttca cctagatcct tttaaattaa |
| 7141 | aaatgaagtt ttaaatcaat ctaaagtata tatgagtaaa cttggtctga cagttaccaa |
| 7201 | tgcttaatca gtgaggcacc tatctcagcg atctgtctat ttcgttcatc catagttgcc |
| 7261 | tgactccccg tcgtgtagat aactacgata cgggagggct taccatctgg ccccagtgct |
| 7321 | gcaatgatac cgcgagaccc acgctcaccg gctccagatt tatcagcaat aaaccagcca |
| 7381 | gccggaaggg ccgagcgcag aagtggtcct gcaactttat ccgcctccat ccagtctatt |
| 7441 | aattgttgcc gggaagctag agtaagtagt tcgccagtta atagtttgcg caacgttgtt |
| 7501 | gccattgcta caggcatcgt ggtgtcacgc tcgtcgtttg gtatggcttc attcagctcc |
| 7561 | ggttcccaac gatcaaggcg agttacatga tcccccatgt tgtgcaaaaa agcggttagc |
| 7621 | tccttcggtc ctccgatcgt tgtcagaagt aagttggccg cagtgttatc actcatggtt |
| 7681 | atggcagcac tgcataattc tcttactgtc atgccatccg taagatgctt ttctgtgact |
| 7741 | ggtgagtact caaccaagtc attctgagaa tagtgtatgc ggcgaccgag ttgctcttgc |
| 7801 | ccggcgtcaa tacgggataa taccgcgcca catagcagaa ctttaaaagt gctcatcatt |
| 7861 | ggaaaacgtt cttcggggcg aaaactctca aggatcttac cgctgttgag atccagttcg |
| 7921 | atgtaaccca ctcgtgcacc caactgatct tcagcatctt ttactttcac cagcgtttct |
| 7981 | gggtgagcaa aaacaggaag gcaaaatgcc gcaaaaaagg gaataagggc gacacggaaa |
| 8041 | tgttgaatac tcatactctt cctttttcaa tattattgaa gcatttatca gggttattgt |
| 8101 | ctcatgagcg gatacatatt tgaatgtatt tagaaaaata aacaaatagg ggttccgcgc |
| 8161 | acatttcccc gaaaagtgcc acctgacgtc gacggatcgg gagatcaact tgtttattgc |
| 8221 | agcttataat ggttacaaat aaagcaatag catcacaaat ttcacaaata aagcattttt |
| 8281 | ttcactgcat tctagttgtg gtttgtccaa actcatcaat gtatcttatc atgtctggat |
| 8341 | caactggata actcaagcta accaaaatca tcccaaactt cccaccccat accctattac |
| 8401 | cactgccaat tacctgtggt ttcatttact ctaaacctgt gattcctctg aattattttc |
| 8461 | attttaaaga aattgtattt gttaaatatg tactacaaac ttagtagt |

TABLE 13
Third nucleic acid construct

| | Length | |
|---|---|---|
| Name | (bp) | Reference |
| pLVX-CMV 100 vector | 2178 | Dean et al., Biophys. J. |
| backbone | | 110(6): 1456-65 (2016) |
| multiple cloning site | 6 | |
| Gal4 Binding site(×4) | 150 | Addgene plasmid # 79130 |
| minCMV | 66 | Addgene plasmid # 79130 |
| connector | 66 | Addgene plasmid # 36083 |
| EGFP | 720 | Addgene plasmid # 36083 |
| multiple cloning site | 28 | |
| PGK promoter | 500 | Addgene plasmid # 79130 |
| connector | 20 | Addgene plasmid # 79130 |
| mCherry | 711 | Addgene plasmid # 79130 |
| multiple cloning site | 24 | |
| pLVX-CMV 100 vector | 4039 | Diagonally Scanned Light-Sheet |
| backbone | | Microscopy for Fast Volumetric Imaging |
| | | of Adherent Cells. Dean KM, |
| | | Roudot P, Reis CR, Welf ES, |
| | | Mettlen M, Fiolka R. Biophys J. 2016 |
| | | Mar 29;110(6): 1456-65. |

### Expression Vectors

The nucleic acids (or nucleic acid constructs) encoding the Protease CAR, the Activation CAR, and the therapeutic payload may be introduced into an immune cell by one or more suitable expression vectors. An expression vector is configured and contains the elements necessary to effect transport into the immune cell and effect expression of the nucleic acid(s) after transformation. Such elements, which are not necessarily included in the disclosed nucleic acid constructs, include an origin of replication, a poly-A tail sequence, a selectable marker, and one or more suitable sites for the insertion of the nucleic acids, such as a multiple cloning site (MCS).

In some embodiments, the expression vector is a viral vector, for example, a retroviral vector, a lentiviral vector, an adenoviral vector, a herpesvirus vector, an adenovirus, or an adeno-associated virus (AAV) vector. As used herein, the term “lentiviral vector” is intended to mean an infectious lentiviral particle. Lentivirinae (lentiviruses) is a subfamily of enveloped retrovirinae (retroviruses), that are distinguishable from other viruses by virion structure, host range, and pathological effects. An infectious lentiviral particle will be capable of invading a target host cell, including infecting, and transducing non-dividing cells and immune cells.

In some embodiments, the expression vector is a non-integrative and non-replicative recombinant lentivirus vector. The construction of lentiviral vectors has been described, for example, in U.S. Pat. Nos. 5,665,577, 5,981,276, 6,013,516, 7,090,837, 8,119,119 and 10,954,530. Lentivirus vectors include a defective lentiviral genome, i.e., in which at least one of the lentivirus genes gag, pol, and env, has been inactivated or deleted.

In other embodiments, the expression vector is a non-viral vector, representative examples of which include plasmids, mRNA, linear single stranded (ss) DNA or linear double stranded (ds) DNA, minicircles, and transposon-based vectors, such as Sleeping Beauty (SB)-based vectors and piggyBac (PB)-based vectors. In yet other embodiments, the vector may include both viral and non-viral elements.

In some embodiments the vector is a plasmid. In addition to a promoter operatively linked to the nucleic acids, the plasmid may also contain other elements e.g., that facilitate transport and expression of the nucleic acid in an immune cell. The plasmid may be linearized with restriction enzymes, in vitro transcribed to produce mRNA, and then modified with a 5′ cap and 3′ poly-A tail. In some embodiments, the vector multiple plasmids, a first plasmid encoding the Protease CAR with the nucleic acid sequence set forth in SEQ ID NO: 84 and the features set forth in Table 9, a second plasmid encoding the Activation CAR with the nucleic acid sequence set forth in SEQ ID NO: 97 and the features set forth in Table 10, and a third plasmid encoding the third nucleic acid with the nucleic acid sequence set forth in SEQ ID NO: 127 and the features set forth in Table 13.

In some embodiments, a carrier encapsulates the vector. The carrier may be lipid-based, e.g., lipid nanoparticles (LNPs), liposomes, lipid vesicles, or lipoplexes. In some embodiments, the carrier is an LNP. In certain embodiments, an LNP includes two or more concentric bilayers separated by aqueous compartments. Lipid bilayers may be functionalized and/or crosslinked to one another. Lipid bilayers may include one or more ligands, proteins, or channels.

Lipid carriers, e.g., LNPs may include one or more cationic/ionizable lipids, one or more polymer conjugated lipids, one or more structural lipids, and/or one or more phospholipids. A “cationic lipid” refers to positively charged lipid or a lipid capable of holding a positive charge. Cationic lipids include one or more amine group(s) which bear the positive charge, depending on pH. A “polymer conjugated lipid” refers to a lipid with a conjugated polymer portion. Polymer conjugated lipids include a pegylated lipids, which are lipids conjugated to polyethylene glycol. A “structure lipid” refers to a non-cationic lipid that does not have a net charge at physiological pH. Exemplary structural lipids include cholesterol, fecosterol, sitosterol, ergosterol, campesterol and the like. A “phospholipid” refers to lipids that have a triester of glycerol with two fatty acids and one phosphate ion. Phospholipids in LNPs assemble the lipids into one or more lipid bilayers. LNPs, their method of preparation, formulation, and delivery are disclosed in, e.g., U.S. Patent Application Publication Nos. 2004/0142025, 2007/0042031, and 2020/0237679 and U.S. Pat. Nos. 9,364,435, 9,518,272, 10,022,435, and 11,191,849.

Lipoplexes, liposomes, and lipid nanoparticles may include a combination of lipid molecules, e.g., a cationic lipid, a neutral lipid, an anionic lipid, polypeptide-lipid conjugates, and other stabilization components. Representative stabilization components include antioxidants, surfactants, and salts. Compositions and preparation methods of lipoplexes, liposomes, and lipid nanoparticles are known in the art. See, e.g., U.S. Pat. Nos. 8,058,069, 8,969,353, 9,682,139, 10,238,754, U.S. Patent Application Publications 2005/0064026 and 2018/0291086, and Lasic, Trends Biotechnol. 16 (7): 307-21 (1998), Lasic et al., FEBS Lett. 312 (2-3): 255-8 (1992), and Drummond et al., Pharmacol. Rev. 51 (4): 691-743 (1999).

### Cells

One aspect of the present disclosure is a genetically modified immune cell expressing the Protease CAR, the Activation CAR, and the therapeutic payload. As used herein, “immune cell” refers to a cell of hematopoietic origin functionally involved in the initiation and/or execution of innate and/or adaptative immune response. Representative examples of immune cells include T cells, natural killer (NK) cells, and NK T (NKT) cells. Combination of different immune cells may be used. Representative examples of T cells include cytotoxic lymphocytes, cytotoxic T cells (CD8⁺ T cells), T helper cells (CD4⁺ T cells), αβ T cells and/or γδ T cells, and Th17 T-cells. In some embodiments, the immune cells are CD8⁺ T cells. In some embodiments, the immune cells are CD4⁺ T cells. In some embodiments, the immune cells are a combination of CD8⁺ T cells and CD4⁺ T cells. In some embodiments, the immune cells are NK cells. The immune cells may be primary cells isolated from healthy patients and engineered to express a fusion protein and optionally a CAR polypeptide. In some embodiments, the immune cells are human immune cells.

Immune cells include cells derived from stem cells. The stem cells can be adult stem cells (e.g., induced pluripotent stem cells (iPSC)), embryonic stem cells, cord blood stem cells, progenitor cells, bone marrow stem cells, induced pluripotent stem cells, totipotent stem cells or hematopoietic stem cells. In some embodiments, the immune cells are derived from peripheral blood mononuclear cells (PBMC), cell lines, or cell bank cells. The collection, isolation, purification, and differentiation of cells from body fluids and tissues is known in the art. See, for example, Brown et al., PloS One 5: e11373-9 (2010), Rivera et al., Curr. Protoc. Stem Cell Biol. 54: e117-21 (2020), Seki et al., Cell Stem Cell 7:11-4 (2010), Takahashi et al., Cell 126:663-76 (2006), Fusaki et al., Proc. Jpn. Acad. Ser. B Phys. Biol. Sci. 85:348-62 (2009), Park et al., Nature 451:141-6 (2008), and U.S. Pat. Nos. 10,214,722, 10,370,452, 10,428,309, 10,844,356, 11,141,471, 11,162,076, and 11,193,108 and U.S. Patent Application Publications 2012/0121544, 2018/0362927, 2019/0112577, and 2021/0015859.

In some embodiments, the immune cells contain one or more genetic modifications. In some embodiments, the cells are genetically modified by knocking out a component of the T cell receptor (TCR), including one or more of T cell receptor α constant (TRAC), T cell receptor βconstant (TRBC) 1, TRBC2, CD3γ, CD3δ, and CD3ε. In some embodiments, the cells are genetically modified by knocking out one or more of β-2-microglobulin (B2MG), class II major histocompatibility complex transactivator (CIITA), HLA class I, and HLA class II.

Methods of introducing the vectors containing the Protease CAR, Activation CAR and the third nucleic acid into immune cells are known in the art. See, e.g., U.S. Pat. Nos. 7,399,633, 7,575,925, 10,072,062, 10,370,452, and 10,829,735 and U.S. Patent Publications 2019/0000880 and 2021/0407639.

In some embodiments, the method entails lentiviral expression vector transduction into immune cells. In other embodiments, the method entails the use of gamma retroviral vectors. See, e.g., U.S. Pat. Nos. 9,669,049, 11,065,311, and 11,230,719. In some embodiments, the method entails the use of Adenovirus, Adeno-associated virus (AAV), dsRNA, ssDNA, or dsRNA to deliver the first, the second, and the third nucleic acids. See, e.g., U.S. Pat. No. 10,563,226, and U.S. Patent Application Publications 2019/0225991, 2020/0080108, and 2022/0186263.

In some embodiments, the vector containing the nucleic acid sequences is delivered to an immune cell by lipofection. See, e.g., U.S. Pat. Nos. 5,049,386, 4,946,787; and 4,897,355.

In some embodiments, the method entails ex vivo or in vivo delivery of linear, circular, or self-amplifying mRNAs. See, e.g., U.S. Pat. Nos. 7,442,381, 7,332,322, 9,822,378, 9,254,265, 10,532,067, and 11,291,682. In some embodiments, the method entails the use of a transposase to integrate the vector-delivered nucleic acids into the immune cell's genome. See, e.g., U.S. Pat. Nos. 7,985,739, 10,174,309, 11,186,847, and 11,351,272. In some embodiments, the method entails the use of self-replicating episomal nano-vectors. See, e.g., U.S. Pat. Nos. 5,624,820, 5,674,703, and 9,340,775.

### Pharmaceutical Compositions

Pharmaceutical compositions of the disclosure include a therapeutically effective number of the genetically modified immune cells and a pharmaceutically acceptable carrier. The term “therapeutically effective number of immune cells” (which indirectly includes a corresponding amount of the Protease CAR, the Activation CAR, and therapeutic payload) as used herein refers to a sufficient number of the immune cells that contain the nucleic acids to provide the desired effect.

The effective number of the genetically modified immune cells for a given patient varies depending one or more factors that may include the age, body weight, type, location, and severity of the cancer and general health of the subject. Ultimately, the attending physician will decide the appropriate dose and dosage regimen. Typically, the immune cells will be given in a single dose. In some embodiments, the effective number of the genetically modified immune cells is about 1×10⁵ to about 1×10¹⁰ cells per subject. In some embodiments, the effective number of the genetically modified immune cells is about 1×10⁵ to about 6×10⁸ cells per kg of subject body weight.

Compositions may be provided as sterile liquid preparations, e.g., isotonic aqueous solutions, suspensions, emulsions, dispersions, or viscous compositions, which may be buffered to a selected pH. Liquid carriers include aqueous or non-aqueous carriers alike. Representative examples of liquid carriers include saline, phosphate buffered saline, a soluble protein, dimethyl sulfoxide (DMSO), polyol (e.g., glycerol, propylene glycol, liquid polyethylene glycol, and the like) and suitable mixtures thereof. In some embodiments, the liquid carrier includes a protein dissolved or dispersed therein, representative examples include serum albumin (e.g., human serum albumin, recombinant human albumin), gelatin, and casein. The compositions are typically isotonic, i.e., they have the same osmotic pressure as blood. Sodium chloride and isotonic electrolyte solutions (e.g., Plasma-Lyte®) may be used to achieve the desired isotonicity. Depending on the carrier and the immune cells, other excipients may be added, e.g., wetting, dispersing, or emulsifying agents, gelling and viscosity enhancing agents, preservatives and the like as known in the art.

### Methods of Use

In some aspects, the present disclosure is directed to treating cancer in a subject. The method entails administering to a subject in need thereof a therapeutically effective number of the genetically modified immune cells having a nucleic acid encoding the Protease CAR, the Activation CAR, and the therapeutic payload.

The term “subject” (or “patient”) as used herein includes all members of the animal kingdom prone (or disposed) to or suffering from the indicated cancer. In some embodiments, the subject is a human. Therefore, a subject “having a cancer” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to cancer (e.g., on account of the basis of prior medical history and/or family medical history, or who otherwise present with a one or more risk factors such that a medical professional might reasonably suspect that the subject was predisposed to cancer).

The terms “treat”, “treating”, and “treatment” as used herein refer to any type of intervention, process performed on, or the administration of the genetically modified immune cells to the subject in need thereof with the therapeutic objective (“therapeutic effect”) of reversing, alleviating, ameliorating, inhibiting, diminishing, slowing down, arresting, stabilizing, or preventing the onset, progression, development, severity or recurrence of a symptom, complication or condition, or biochemical indicia associated with a cancer.

In some embodiments, the cells are allogeneic to the subject receiving the cells, that is, the cells have a complete or at least partial HLA-match with the subject. In some embodiments, the cells are autologous. The term “autologous” as used herein refers to any material (e.g., T cells or NK cells) derived from the same subject to whom it is later re-introduced. The term “allogeneic” as used herein refers to any material derived from a different subject of the same species as the subject to whom the material is later introduced. Two or more individual subjects are allogeneic when the genes at one or more loci are not identical (typically the HLA loci).

In some embodiments, the cancer is characterized by a solid tumor. Representative cancers characterized by a solid tumor include breast cancer, bladder cancer, ovarian cancer, pancreatic cancer, lung cancer, hepatic cancer, or prostate cancer.

In some embodiments, the cancer is a hematological cancer. Representative hematological cancers include plasma cell neoplasm (e.g., myeloma, multiple myeloma, relapsed or refractory multiple myeloma, plasma cell myeloma, extramedullary multiple myeloma, monoclonal gammopathy of unknown significance (MUGS), asymptomatic smoldering multiple myeloma, or solitary plasmacytoma), lymphoma (e.g., Hodgkin's lymphoma, non-Hodgkin's lymphoma, Burkitt's lymphoma, plasmablastic lymphoma, plasmacytoid lymphoma, or diffuse large B-cell lymphoma), leukemia (e.g., relapsed or refractory acute B lymphocytic leukemia, or relapsed or refractory acute lymphoblastic leukemia), and carcinomas (e.g., Waldenstrom macroglobulinemia or glioblastoma (astrocytoma)). In these embodiments, the therapeutic effect might include on or more art-recognized indicia of therapeutic efficacy, representative examples of which include prevention or prolongation of metastases, improvement in survival time, total/complete or partial remission of a cancer, e.g., no detectable cancer cells and less tumor cells or smaller tumors, respectively, or a reduction in tumor cell number.

In some embodiments, the hematological cancer is multiple myeloma, leukemia, or lymphoma. In some embodiments, the hematological cancer is multiple myeloma and the first and second antigen binding domains bind BCMA, CD19, CD38, CD138, GPCR5D, FCHR5, SLAMF7, or a combination thereof. In some embodiments, the hematological cancer is leukemia or lymphoma and the first and second antigen binding domains bind CD19, CD20, CD33, CD38, FCHR5, Flt3, or a combination thereof.

### Combination Therapy

In some embodiments, the present methods may include co-administration of an anti-cancer agent.

The terms “co-administration”, “co-administer” and co-administered” include substantially contemporaneous administration, by the same or separate dosage forms, or sequentially, e.g., as part of the same treatment regimen or by way of successive treatment regimens. Thus, if given sequentially, at the onset of administration of the second therapy, the first of the two therapies is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

Anti-cancer agents that may be used in combination with the inventive cells are known in the art. See, e.g., U.S. Pat. No. 9,101,622 (Section 5.2 thereof). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of cancerous cells. This process may involve contacting the cancer cells with recipient cells and the agent(s) or multiple factor(s) at the same time. This may be achieved by contacting the cancer cells with a single composition or pharmacological formulation that includes both agents, or by contacting the cancer cells with two distinct compositions or formulations, at the same time, wherein one composition includes recipient cells and the other includes the second agent(s). In some embodiments, the genetically modified immune cells of the present

disclosure are used in conjunction with chemotherapeutic, radiotherapeutic, immunotherapeutic intervention, targeted therapy, pro-apoptotic therapy, or cell cycle regulation therapy.

### Immunotherapy

Immunotherapy, including co-administration of immune checkpoint inhibitors may be employed to treat a cancer. Immune checkpoint molecules include, for example, PD1, CTLA4, KIR, TIGIT, TIM-3, LAG-3, BTLA, VISTA, CD47, and NKG2A. Clinically available examples of immune checkpoint inhibitors include durvalumab (Imfinzi®), atezolizumab (Tecentriq®), and avelumab (Bavencio®). Clinically available examples of PD1 inhibitors include nivolumab (Opdivo®), pembrolizumab (Keytruda®), and cemiplimab (Libtayo®).

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, Taxol®, gemcitabien, Navelbine®, farnesyl-protein tansferase inhibitors, transplatinum, 5-fluorouracil, vincristine, vinblastine and methotrexate, or any analog or derivative variant of the foregoing and also combinations thereof.

### Radiotherapy

Anti-cancer therapies also include radiation-based, DNA-damaging treatments. Combination radiotherapies include what are commonly known as gamma-rays, X-rays, and/or the directed delivery of radioisotopes to tumor cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

Radiotherapy may include external or internal radiation therapy. External radiation therapy involves a radiation source outside the subject's body and sending the radiation toward the area of the cancer within the body. Internal radiation therapy uses a radioactive substance sealed in needles, seeds, wires, or catheters that are placed directly into or near the cancer.

These and other aspects of the present disclosure will be further appreciated upon consideration of the following working examples, which are intended to illustrate certain embodiments of the disclosure but are not intended to limit its scope, as defined by the claims.

### Example 1: Materials and Methods

cCAR cells were produced using vector and lentiviral infection. Third generation (CAR) constructs (see Table 9 and Table 10) antigen binding domains containing single chain variable fragments targeting the BCMA antigen with an intracellular domain containing CD3ζ primary signaling domain as well as 4-1BB and CD28 co-stimulatory domains were designed; all expressed under the control of an EF-1α promoter. The Protease CAR contained a MYC-tag and the delta220-242 S219V TEV protease separated by a GGGS linker (SEQ ID NO: 90); the Activation CAR contained the TEV protease cleavage site (ENLYFQM (SEQ ID NO: 83)), the transcriptional activator GAL4-VP64, and a truncated epidermal growth factor receptor (tEGFR), separated from the CAR by a T2A sequence. The third nucleic acid (see Table 13) contained four repeats of the Gal4 Binding site followed by a minimal CMV promoter and an enhanced green fluorescent protein (eGFP) reporter protein as an inducible payload proxy and a mCherry fluorescent tag to identify successful integration of the construct.

293T cells were co-transfected with the Protease CAR and Activation CAR lentiviral construct, psPAX2 and pCMV-VSV-G packaging vectors using Lipofectamine 3000, commercially available from Thermo Fisher Scientific, according to manufacturer's protocol. Lentivirus was collected and medium was exchanged after 12, 24, and 36 hours. The virus was concentrated by filtration and ultracentrifugation for 2 h at 20,000 rpm at 4° C.

T-cell isolation and transduction. T cell experiments were performed either with Jurkat cells or primary human T cells. Human blood from healthy donors was obtained from Research Blood Components, LLC or the Crimson Core of the Brigham and Women's Hospital. Mononuclear cells (PBMCs) were isolated by Ficoll-Paque PLUS (Global Life Sciences Solutions USA LLC). PBMCs were further processed by isolating CD3″ T cells with the EasySep™ Human T Cell Enrichment Kit (STEMCELL Technologies). For CD8″ or CD4″ T cell purification, selection was performed using EasySep™ Release Human CD4 or CD8 Positive Selection Kit (STEMCELL Technologies) according to manufacturer's protocol.

Isolated T cells were activated by Dynabeads™ Human T-Activator CD3/CD28 (Thermo Fisher Scientific) and cultured in X-VIVO 15 Media (Lonza) supplemented with 5% Human Serum (Sigma-Aldrich). Fifty (50) IU/ml IL-2 (Miltenyi Biotec) was added every other day. One day after isolation, T-cells or Jurkat cells were infected by spinoculation at MOI of 5. After 7 days, infection efficiencies were determined by flow cytometry using an anti-hEGFR antibody (Biotinylated, Cetuximab; R&D Systems), anti-myc antibody and gating on mCherry+ cells. CAR-expressing cells were isolated by magnetic isolation using the EasySep™ Release Human Biotin Positive Selection Kit (STEMCELL Technologies). Activation beads were removed after 10 days with restimulations according to manufacturer's protocol. Uninfected T-cells from the same donor or uninfected Jurkat cells were maintained in parallel and used as controls.

Live cell microscopy imaging was performed in X-VIVO 15 media without phenol red (Lonza) supplemented with 5% Human Serum (Sigma-Aldrich). Tumor cells were stained with CFSE. A stage top incubator was used to maintain constant humidified O₂ and CO₂ flow at 37° C. (Okolab). 10⁶ cells were seeded on a petri dish and allowed to settle for at least 30 min before timelapse imaging. CAR T-cells were carefully added at an approximately 1:1 ratio. Where applicable, SYTOX Blue Dead Cell Stain (Thermo Fisher Scientific) was added to the media at 1 mM.

For holotomography based three-dimensional live microscopy, interaction sites were recorded by measurement of refractive index and CFSE fluorescence signal using a 3D Cell Explorer microscopy system on a 60× magnifying objective at 512×512 resolution (Nanolive). Images were further processed with Nanolive's software STEVE v1.6.3496 to display three dimensional timelapses.

To visualize clustering at the immunological synapse, a Nikon Eclipse Ti microscope system was used to record interaction sites every 10 min for 6 h with a 20× magnifying objective at 2048×2048 resolution. Z-stack images were recorded focusing on the middle layer of the cells as well as 2 μm above and below using Nikon's Perfect Focus System. Dead cells were determined by positivity of SYTOX Blue Stain. Typically, analysis only included tumor cells that are SYTOX Blue negative at time of analysis.

Flow cytometry was performed by the method of co-culturing 10⁵ target OMP2 (Target, T) cells with cCAR T-cells or cCAR Jurkat cells (Effector, E) at an E:T ratio of 1:1, 2:1 and 5:1 in a 96-well round bottom plate for 1, 2, 4, 6, 12 and 24 h. Cells were stained with anti-MYC APC (9B11 Mouse mAb, Cell Signaling Technology), anti-tEGFR PE (Recombinant Monoclonal Human IgG1 Clone #Hu1, R&D Systems; PE Streptavidin, BD Biosciences) or Human EGFR biotinylated Antibody, Recombinant Monoclonal Human IgG1 Clone #Hu1 (R&D systems, Cat #FAB9577B-100), with secondary stain PE Streptavidin (BD Biosciences, CAT #554061) and analyzed on a Fortessa Flow Cytometer (BD Biosciences) with compensation being performed by AbC™ Total Antibody Compensation Bead Kit (Thermo Fisher Scientific). Where applicable, absolute counts were measured with Precision Count beads (BioLegend). Flow cytometry analyses were performed on FlowJo V10 (BD Biosciences).

The following nucleic acid constructs were made, namely: a first nucleic acid construct containing a pLVX-CMV 100 vector backbone, having the sequence of SEQ ID NO: 84 (summarized in Table 9); a second nucleic acid construct having sequence of SEQ ID NO: 97 (summarized in Table 10); and a third nucleic acid construct having sequence of SEQ ID NO: 127 (summarized in Table 13).

### Example 2: Preparation of cCAR Cells

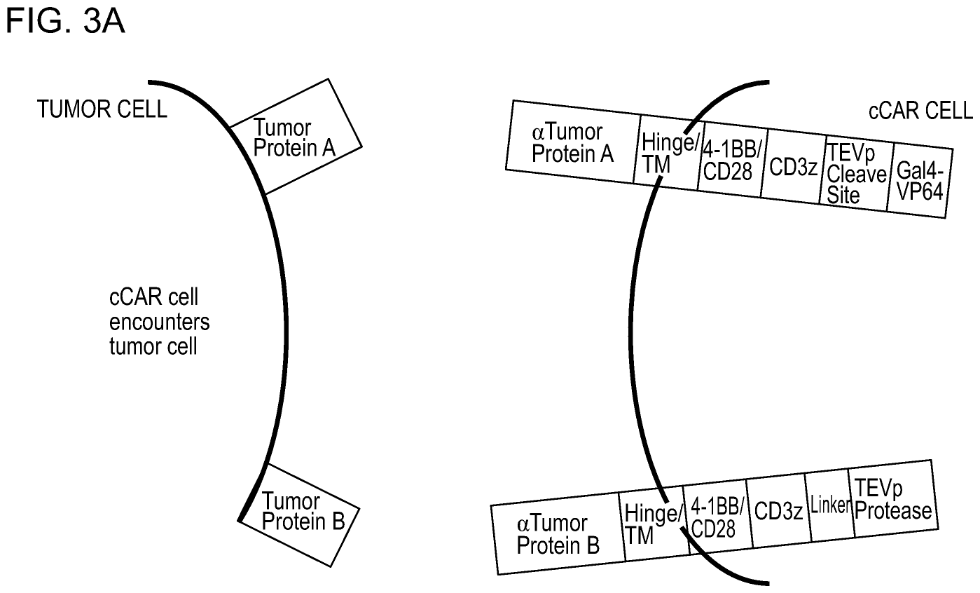

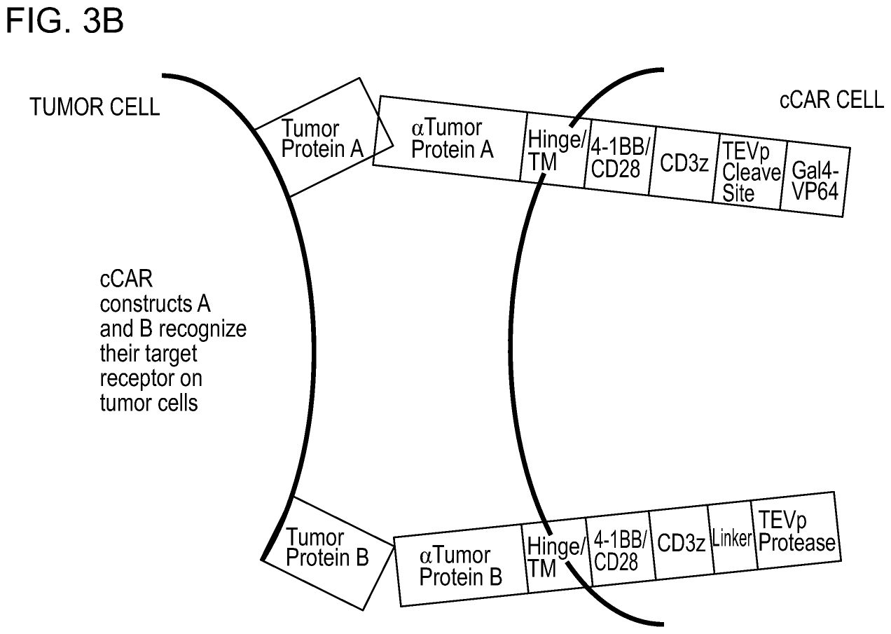

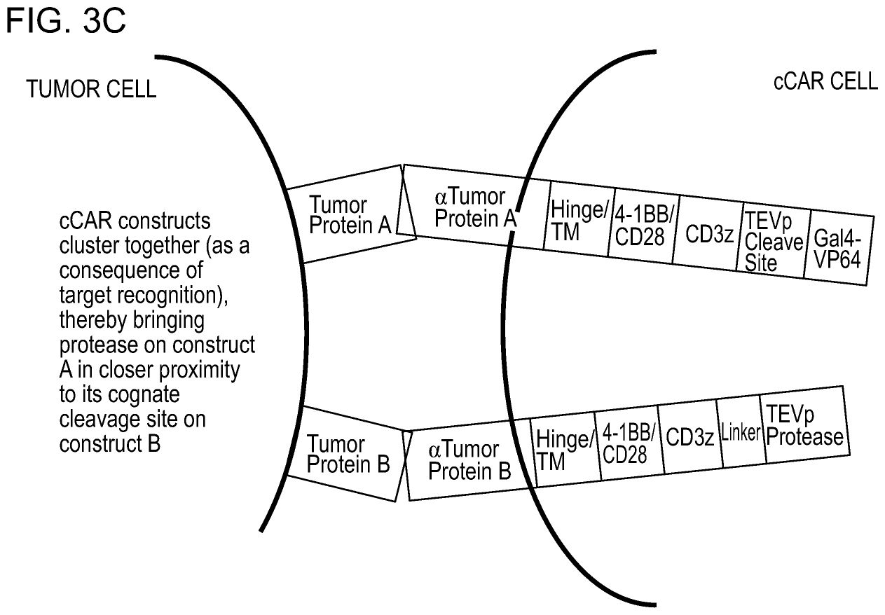

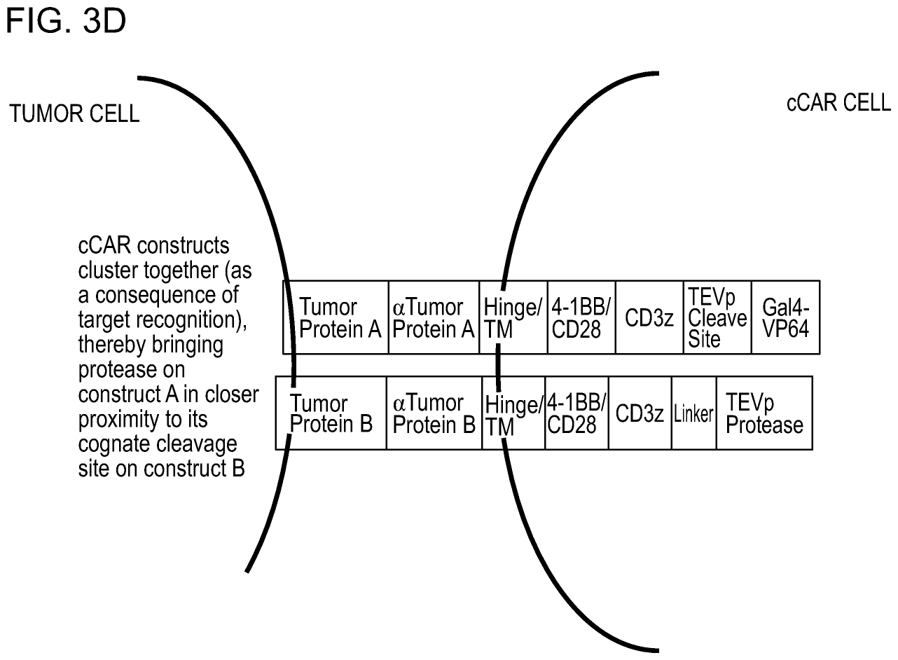

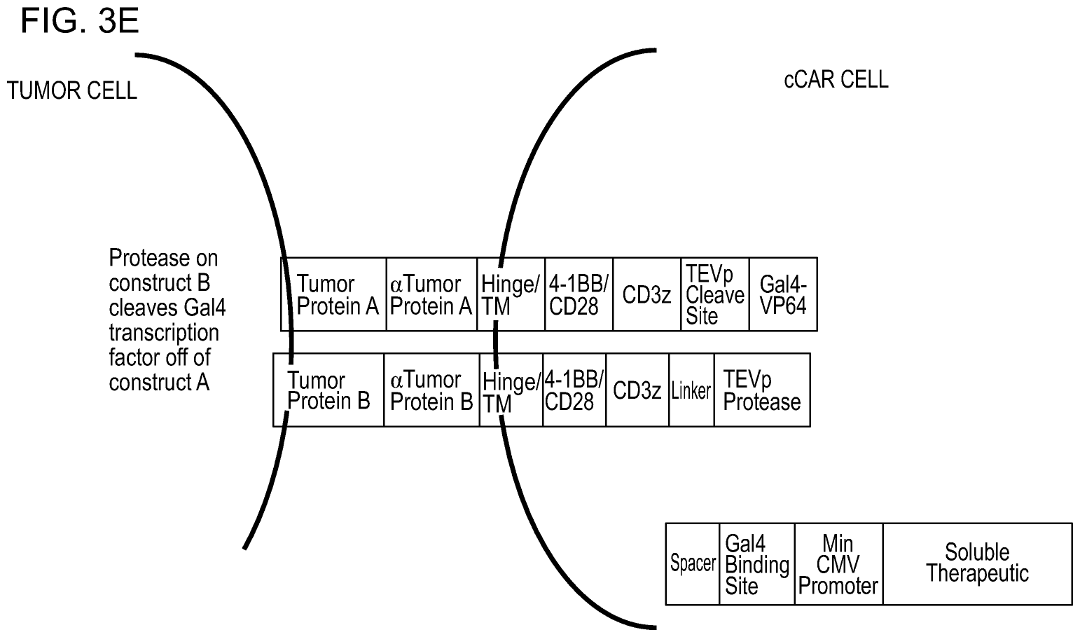

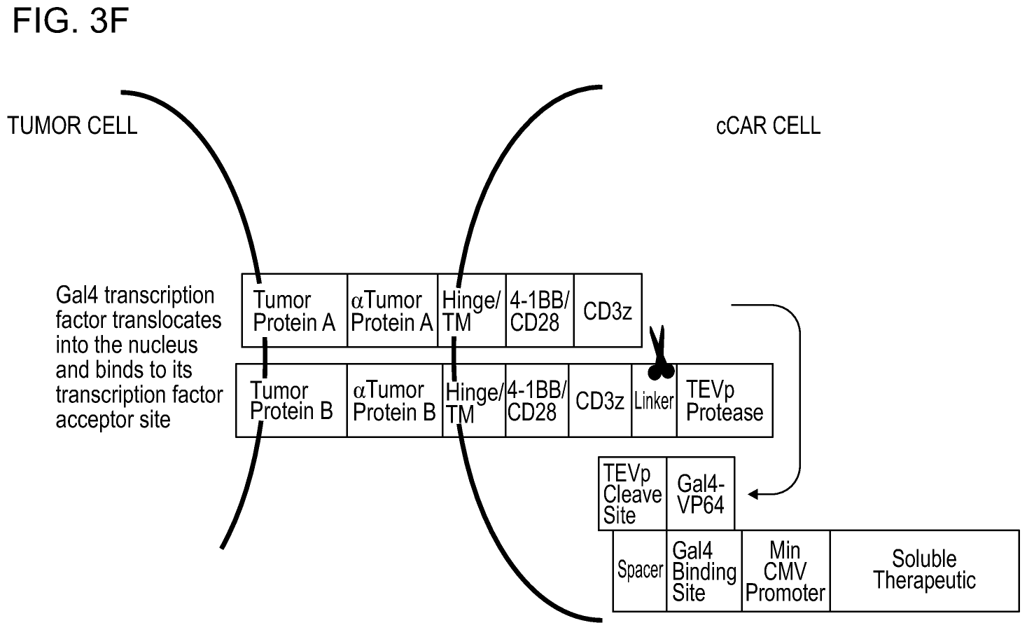

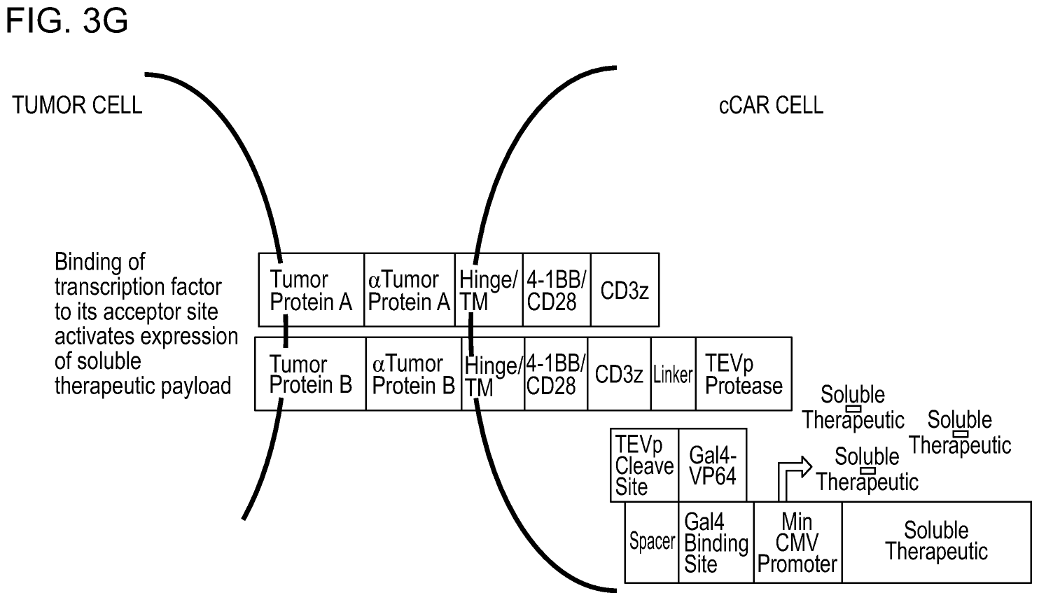

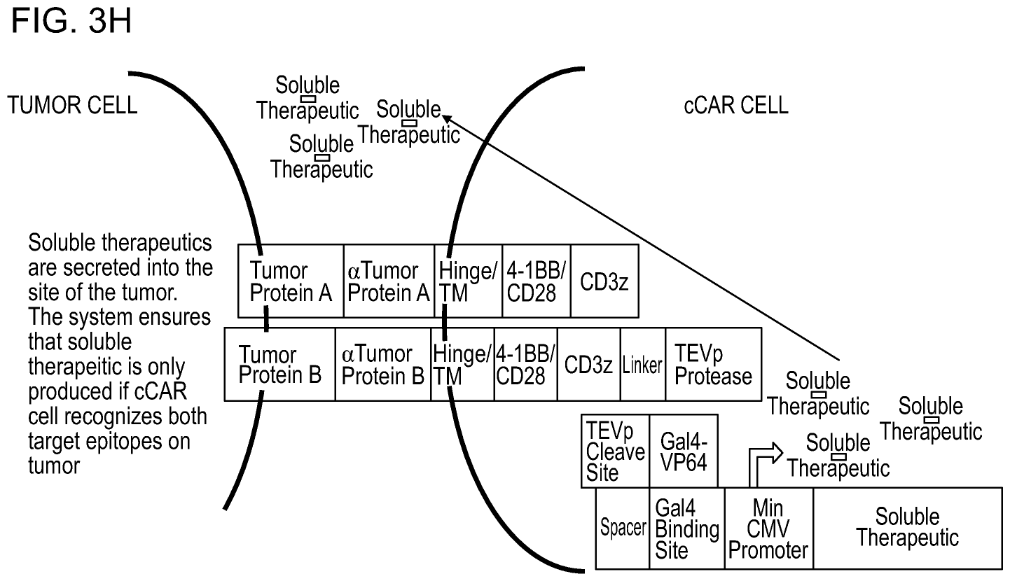

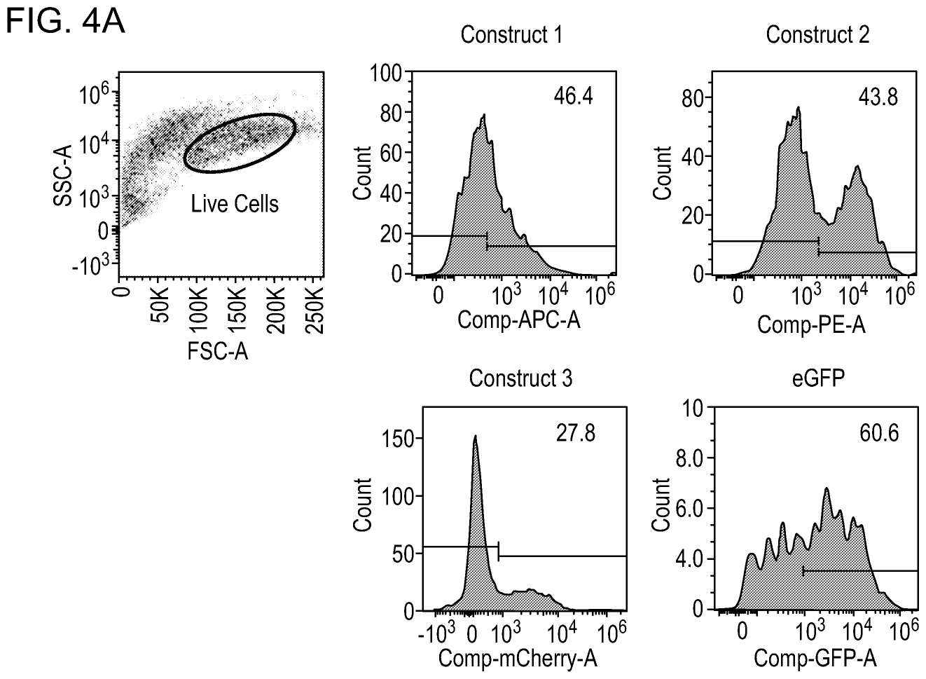

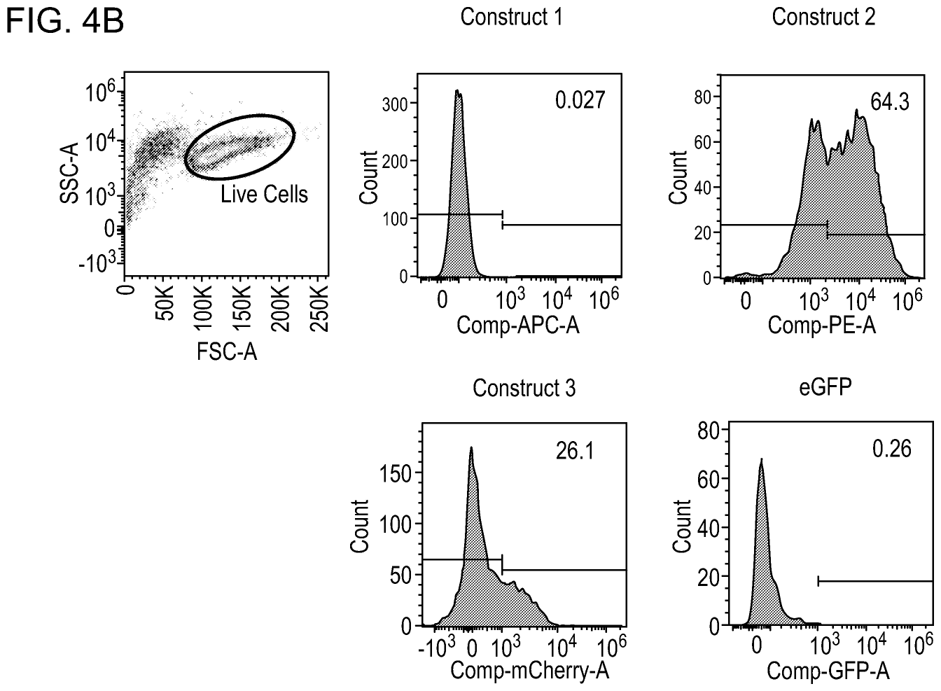

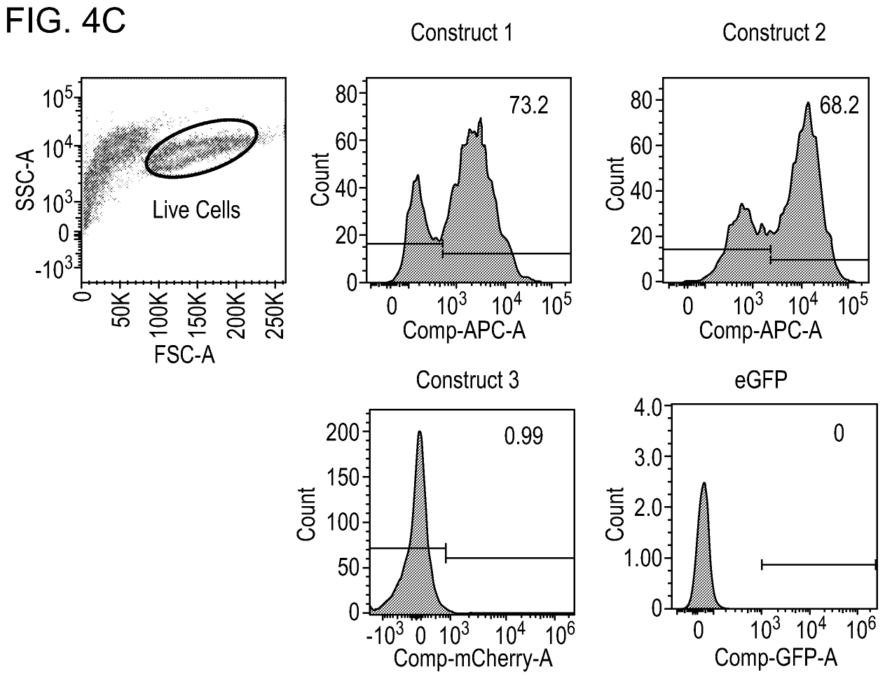

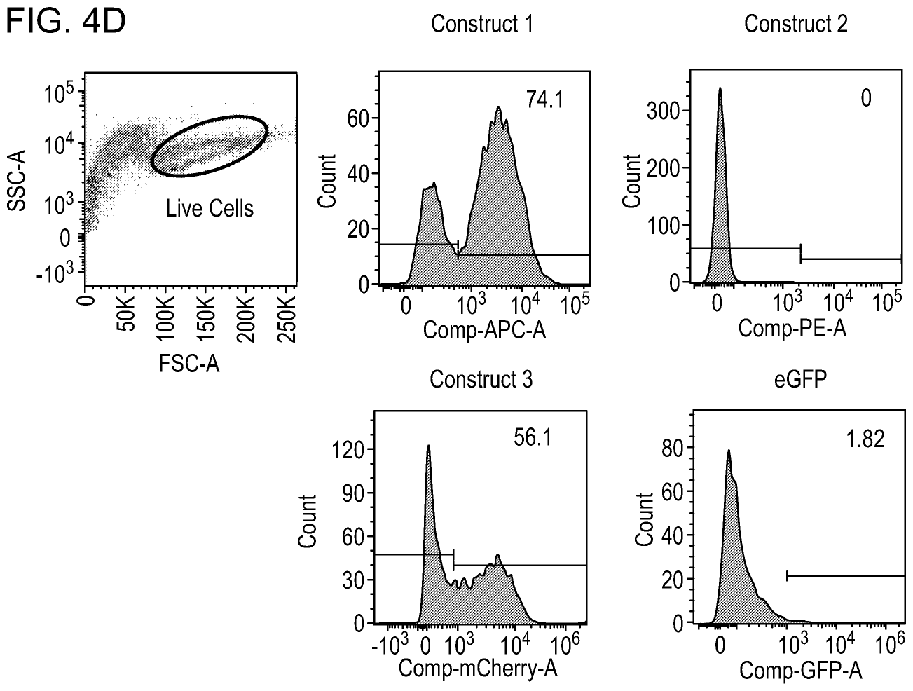

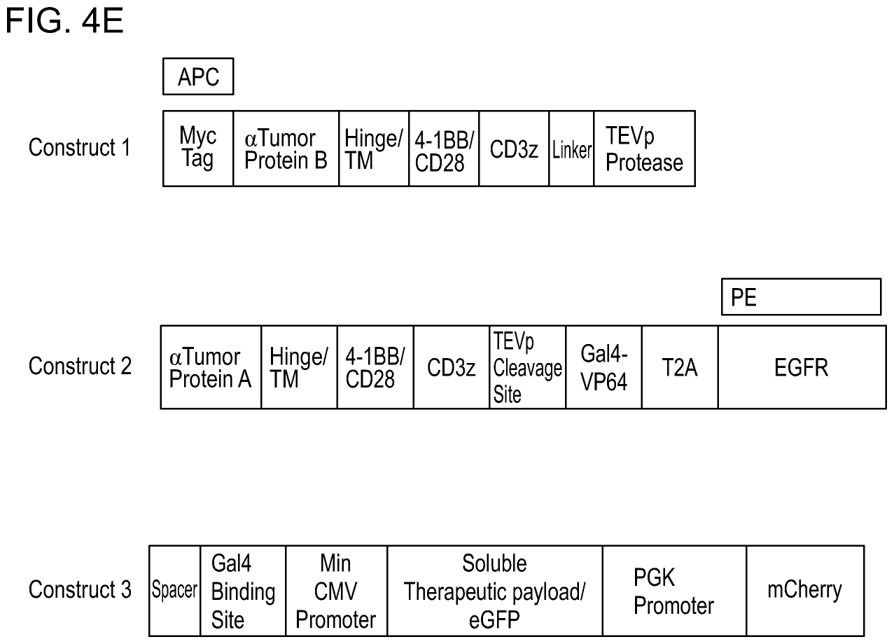

A nucleic acid construct containing three nucleic acids was engineered, including a nucleic acid encoding a Protease CAR with an anti-tumor protein A antigen biding domain, a nucleic acid encoding an Activation CAR with an anti-tumor protein B antigen binding domain and a third nucleic acid encoding a model payload protein of enhanced green fluorescent protein (eGFP), which is expressed when the cCAR cell encounters a cancer cell expressing the Tumor Protein A and Tumor Protein B (i.e., the first and second TAAs), as illustrated in FIGS. 3A-4D, summarized in Table 9 (SEQ ID NO: 84), and Table 10 (SEQ ID NO: 97), respectively. The antigen binding domains of the Protease CAR and the Activation CAR contained scFv fragments of antibodies targeting either anti-Tumor Protein A or Protein B with an extracellular linker and transmembrane domain linked to intracellular domain containing the CD28 or 4-1BB and CD33 signaling domains, as illustrated in FIG. 3A. These signaling domains mediated CAR-specific killing. In the Protease CAR, the signaling domain was followed by a TEV protease, in the Activation CAR, the signaling domain was followed by the corresponding cleavage site of TEV protease, which was fused to the Gal4-VP64 transcriptional activator/transcriptional activator FIG. 3A.

As schematically shown in FIGS. 3B-H, recognition of tumor proteins A and B on the surface of the same tumor cell co-localize the Protease CAR and the Activation CAR, and form an immunological synapse, bringing the intracellular TEV protease and its corresponding cleavage site into close proximity of one another (FIGS. 3B-3D). As a consequence, the Gal4-VP64 transcriptional activator is cleaved away from the rest of the protein (FIGS. 3E and 3F) and allows for translocation into the nucleus (FIG. 3F). In the nucleus, the Gal4-VP64 transcription factor binds its transcriptional acceptor encoded by the third nucleic acid, which is simultaneously introduced into the cell with the other two nucleic acids (FIG. 3F). The third nucleic acid encoding the transcriptional acceptor, here Gal4-VP64 transcription factor acceptor site, which controls transcription of the therapeutic payload. Upon binding of the Gal4-VP64 transcription factor to the transcriptional acceptor, the inducible therapeutic payload is transcribed under the control of a modified CMV promoter and translated into protein (FIG. 3G). The nucleic acid sequence of the therapeutic payload protein was preceded by a leader peptide that targets the therapeutic payload protein to a desired location, e.g., the extracellular environment. The therapeutic payload is therefore secreted into the neighboring environment of the cell (FIG. 3H).

The cCAR cells were assayed by Flow cytometry. The cCAR cells only expressed the model therapeutic payload protein, here eGFP, when both CAR constructs (the Protease CAR and the Activation CAR) recognized and bound to their target TAA with minimal baseline expression (less than 1%), as illustrated in FIG. 4A-4D.

The therapeutic concept disclosed herein has several novel features and represents a significant advance over existing immunotherapeutic concepts as detailed in the following: 1) the cCAR system as disclosed comprises a cellular ON-switch to deliver a therapeutic payload by exploiting the property of heterotypic receptors to coalesce at the cell-cell interface (immunological synapse). 2) The cCAR system provides excellent specificity since immunotherapeutic payload delivery is initiated only when two different target TAAs are expressed on the same tumor cell. This added specificity mitigates toxicity by sparing normal tissues. 3) Once triggered, the secreted payload kills tumor cells in the cluster even if the two different target tumor surface proteins for CAR-specific killing are absent. This ensures that an entire cluster of tumor cells is eliminated (field effect), even if some cells in the cluster have become resistant to CAR-mediated killing through loss of target epitope expression. The system thus overcomes CAR cell resistance. 4) The cCAR system can also be used to deliver therapeutics with exquisite specificity to defined sites in the body without CAR-mediated killing (i.e., embodiments where the Protease CAR and the Activation CAR do not contain signaling domains), allowing its use as an immunotherapeutic delivery system for example in cancer.

### Example 3: Anti-BCMA cCAR Cells to Treat Multiple Myeloma

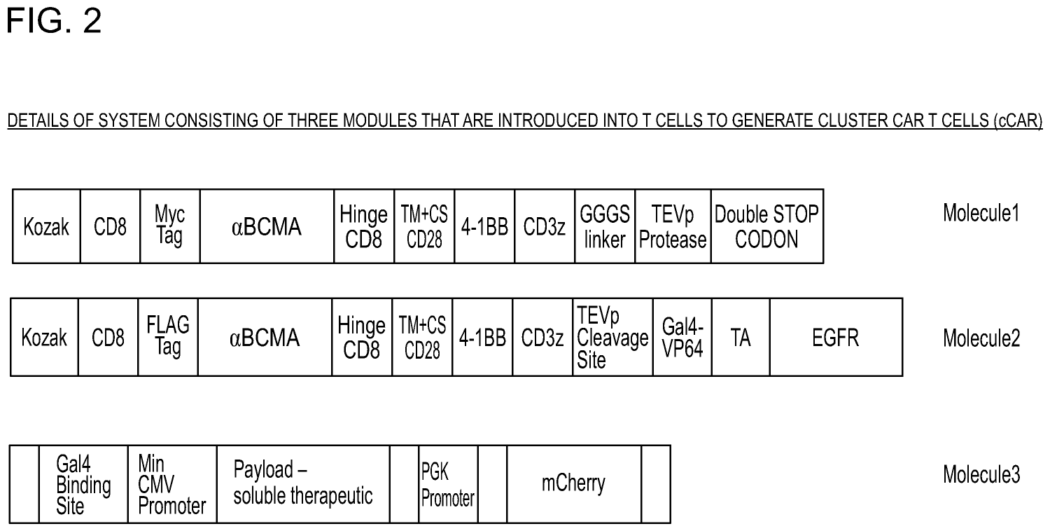

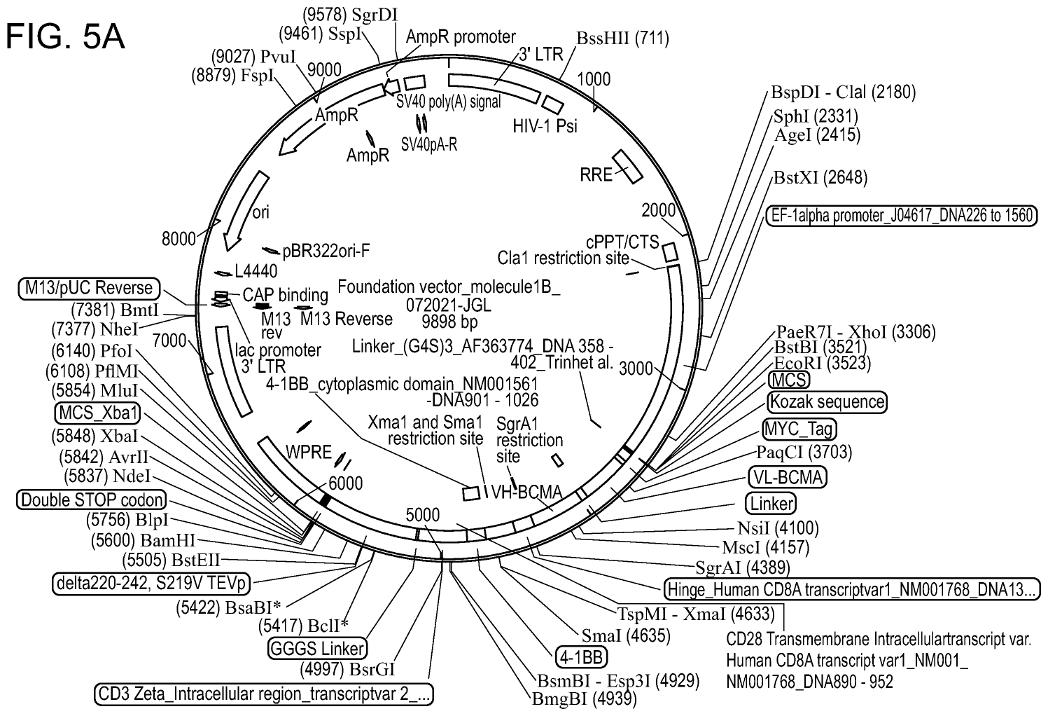

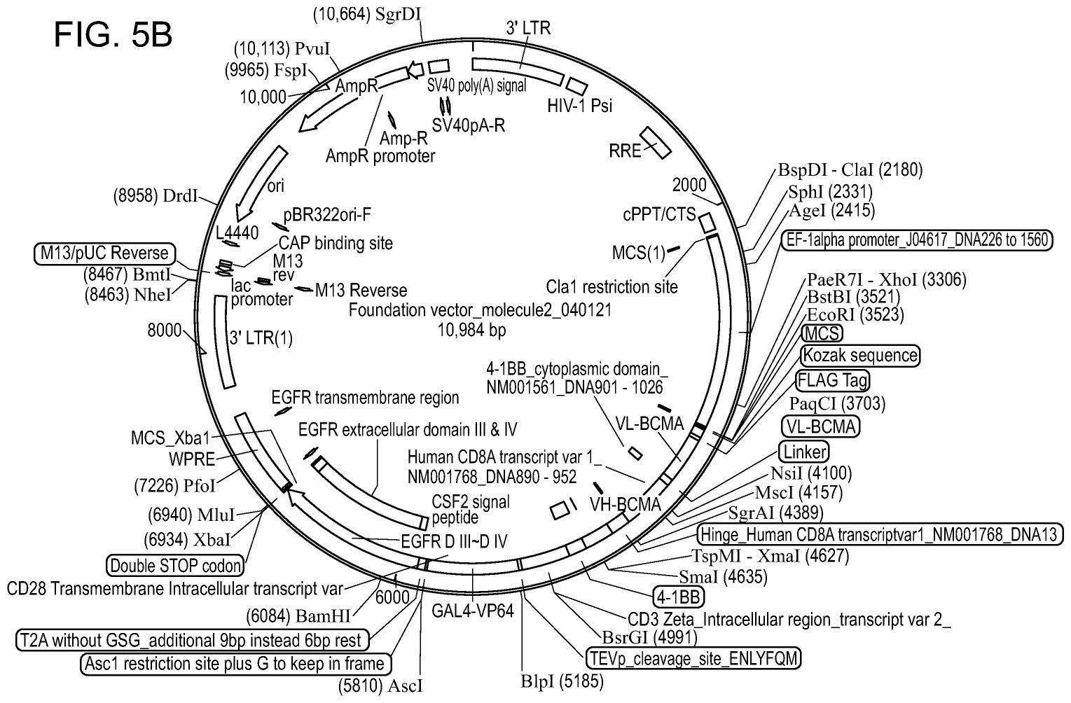

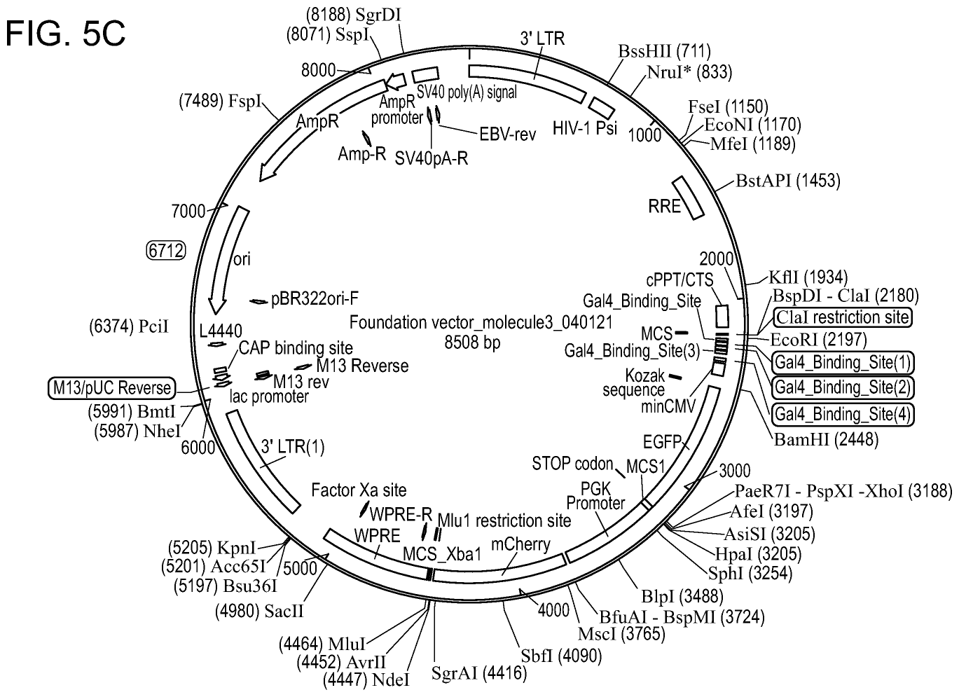

Multiple myeloma, an incurable hematologic malignancy, was chosen for initial proof-of-concept studies. The cCAR system targeted BCMA as anti-Tumor Protein A and B for both of the antigen binding domains of the Protease CAR and the Activation CAR (FIG. 2) and enhanced green fluorescent protein (eGFP) was used as a proxy for a therapeutic payload. Anti-BCMA CAR T cells have excellent activity against myeloma cells, which express high levels of BCMA, and have been FDA-approved in patients with relapsed/refractory myeloma. cCAR cells were infected with all three nucleic acids of the cCAR system, co-cultured with BCMA-expressing myeloma cells (e.g., the OPM2 cell line), and about 80% of the cCAR cells produced high levels of the eGFP payload, as illustrated in FIG. 4A. All three components of the cCAR system were required for effective eGFP production and there was no significant background production of eGFP in the absence of the Protease CAR-encoding construct (<1%), FIGS. 4B-4D. The anti-BCMA Protease CAR and anti-BCMA Activation CAR constructs conferred CAR-mediated myeloma cell killing. The domains of the nucleic acids are shown in more detail in FIGS. 2, and 4E-5C. From left to right, the flow cytometry plots of FIGS. 4A-4D show cCARs stained for the Protease CAR, the Activation CAR, the third nucleic acid, and eGFP, respectively. Jurkat T cells were transformed with lentivirus comprising all three nucleic acids in FIG. 4A. In FIG. 4B cells were infected with virus containing only the Activation CAR and the third nucleic acid, the Protease CAR and the Activation CAR in FIG. 4C, and the Protease CAR and the third nucleic acid in FIG. 4D.

The Protease CAR and the Activation CAR were both directed against the BCMA surface protein (i.e., the Protease CAR and the Activation CAR bind BCMA). For these experiments each construct was engineered to include a marker for detecting construct expression. The Protease CAR includes a sequence for a myc-tag that was stained with α-myc-APC. The Activation CAR includes a sequence for a truncated EGFR receptor that was stained with α-EGFR-PE. The third nucleic acid includes a sequence for the mCherry fluorophore under the control of a PGK promoter. Cells were subsequently co-cultured with OPM2 myeloma cells that express BCMA as the CAR-target on their surface. After 24 hours of co-culture flow-cytometry was performed, gating on live cells that were transfected with the three nucleic acids (i.e., APC⁺PE⁺mCherry⁺ in FIG. 4A). As can be seen in FIG. 4A, the majority of the gated cells express eGFP as the model payload. Notably, only minimal eGFP payload expression is detected if only 2 of the 3 nucleic acids were transfected and expressed, FIGS. 4B-4D.

### Example 4: cCAR T Cells that Bind BCMA Kill BCMA-Expressing OPM2 Multiple Myeloma Cells

T cells or CAR T cells that were lentivirally infected with the third nucleic acid encoding the therapeutic payload (labeled “3” in FIG. 6), and one or both of α-BCMA Protease CAR and Activation CAR (labeled “1” and “2,” respectively in FIG. 6), were co-cultured at a 2:1 effector to target ratio with OPM2 multiple myeloma cells for 40 hours. The ratio of live OPM2 cells to count beads was normalized to untransfected T cell co-cultures, and two representative experiments, separated by the dashed line, are illustrated in FIG. 6. In both experiments, CAR T cells that were transduced with either the Protease CAR and the Activation CAR, in addition to the third nucleic acid encoding the therapeutic payload, were effective at killing multiple myeloma cells (0.15 and 0.19 Live OPM2/Count Beads, respectively). CAR T cells transduced with two α-BCMA CAR constructs (the Protease CAR and the Activation CAR or “1 and 2”) and the payload carrying the third nucleic acid (“3”) killed myeloma cells (0.13 Live OPM2/Count Beads) similarly to CAR T cells transduced with a single α-BCMA CAR construct (i.e., the Protease CAR or the Activation CAR). These data demonstrate that T cells expressing either the α-BCMA-directed the Protease CAR or the Activation CAR killed BCMA-expressing myeloma cells.

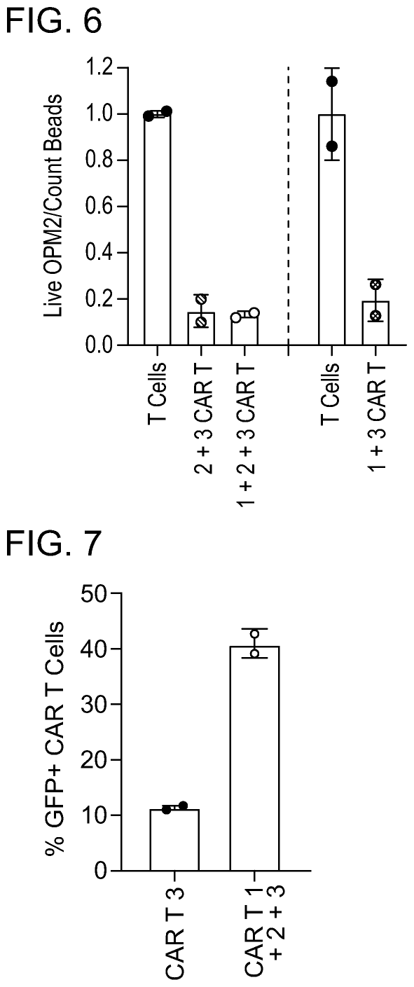

T cells were lentivirally infected with either the third nucleic acid encoding the therapeutic payload alone, or in addition to α-BCMA Protease CAR and α-BCMA Activation CAR (FIG. 7). The infected T cells were co-cultured at a 1:1 effector to target cell ratio with BCMA-expressing OPM2 multiple myeloma cells for 40 hours. GFP was used as a mock therapeutic payload to assess rate of payload transcription and expression. Background therapeutic payload expression (11.3% GFP+ CAR T cells) was seen in T cells that only carry the third nucleic acid. Significant increase of payload expression (40.9% GFP+ CAR T cells) is observed in CAR T cells that express all of the Protease CAR and the Activation CAR and third nucleic acid encoding the therapeutic (FIG. 7). OPM2 target cell death correlated with therapeutic payload expression.

### Example 5: Efficacy of cCAR Cells Against a Multiple Myeloma In Vitro Model

In one embodiment, cCAR cells are generated by simultaneous lentiviral infection to produce cells that express a Protease CAR that binds the CD38 antigen, and an Activation CAR that binds BCMA. The cells also contain the third nucleic acid encoding one or both of the BiTE CD3-CD19, and the BiTE CD3-CD20. These cCAR cells should recognize cancer cells expressing CD38 and BCMA, cluster around these cells, and initiate transcription of the BiTE(s). The BiTE will be secreted due to the adjacent leader peptide for extracellular secretion that will be introduced as part of the third nucleic acid.

In this embodiment, the therapeutic payload encodes the bispecific antibody or bispecific T cell engager. This therapeutic payload comprises a leader peptide that ensures extracellular secretion of the payload protein. Bispecific antibody/bispecific T cell engagers (BiTEs) link cancer cells with T cells, activating the T cells to exert cytotoxic activity on the linked cancer cell. As proof of principle, two different specificities will be tested by introducing a BiTE against CD3-CD19 and against CD3-CD20 as two different therapeutic payloads. Both CD19 and CD20 are known to be expressed on a small subset of myeloma cells. Bispecific T cell engagers or bispecific antibodies have been FDA approved for CD3-CD19 (blinatumomab) or are currently undergoing clinical trials for CD3-CD20 (odronextamab). These bispecific T cell engagers or bispecific antibodies can be tested for efficacy as a payload molecule(s) with cell lines. In one exemplary embodiment of therapeutic payload efficacy, Molp2 myeloma cells may act as target cancer cells, which express CD19, but not CD20 on their cell surface. Karpas620 myeloma cells, which express CD20, but not CD19 on their surface may also act as target cancer cells. CRISPR/Cas9 genomic editing can be used to generate Molp2 and Karpas620 myeloma cells that lack expression of CD38 or BCMA or SLAMF7, or a combination of two or all three of these molecules.

To test specificity of the cCAR cells, the above cCAR cells will be co-cultured with Molp2 cells in which CD38 and BCMA have been knocked out with CRISPR/Cas9 genomic editing (Molp2^{CD38 KO/BCMA KO}) in the presence or absence of wildtype Molp2 cells. It is expected that secretion of the CD3-CD19 BiTE therapeutic payload only occurs in the presence of wild-type Molp2 cells, which expresses both surface CD38 and BCMA. It is therefore predicted that killing of the Molp2^{CD38 KO/BCMA KO} myeloma cells only occurs if wild-type Molp2 cells are also present in co-culture. Additional CAR specificities are disclosed herein, e.g., SLAMF7, CD138, CD38, and BCMA.

### Example 6: cCAR Cells Selectively Kill Antigen-Expressing Tumor Cells

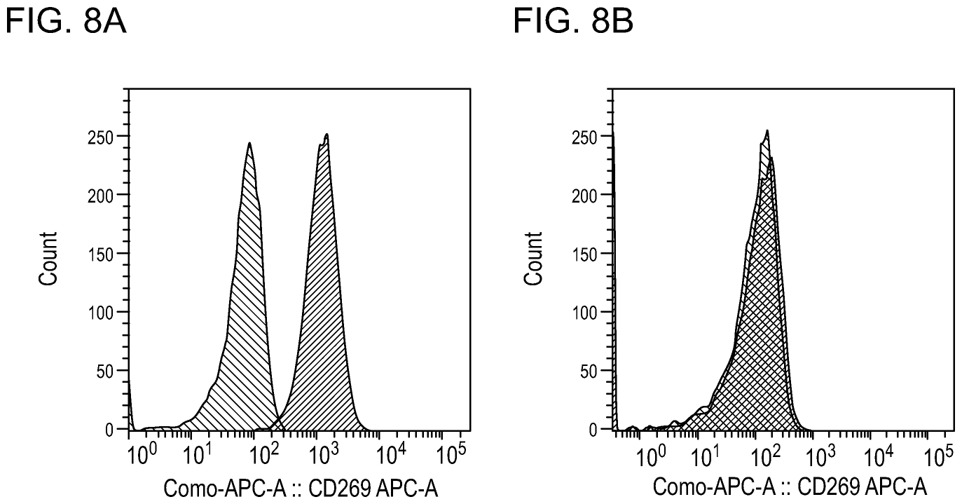

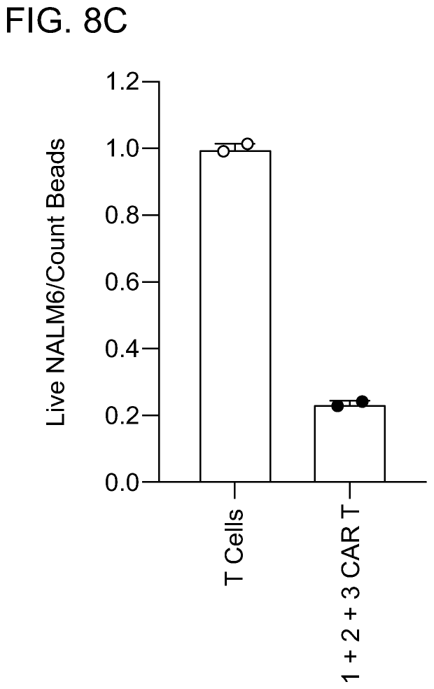

This experiment shows that cCAR cells expressing an anti-BCMA Protease CAR, an anti-BCMA Activation CAR, and a third nucleic acid encoding a CD3/CD19 BiTE selectively killed CD19-positive tumor cells only in the presence of BCMA-positive tumor cells. OPM2 (FIG. 8A) and NALM-6 (FIG. 8B) cancer cells were assessed for BCMA (CD269) expression. Red histogram represents unstained cells while blue represents α-BCMA staining, showing that OPM2 cells (FIG. 8A) were BCMA positive while NALM-6 cells (FIG. 8B) were BCMA negative. cCAR T cells were lentivirally infected with an anti-BCMA Protease CAR, an anti-BCMA Activation CAR, and a third nucleic acid encoding a CD3/CD19 BiTE (labeled 1+2+3 CAR T in FIG. 8C). These cCAR T cells or non-infected control T cells (T cells) were co-cultured with both BCMA-negative/CD19-positive NALM-6 cells and BCMA-positive OPM2 cells at a 2:1 effector to target ratio for 40 hours. CD3/CD19 BiTE expression and killing efficacy is shown as the ratio of live NALM-6 cells to count beads, normalized to killing by non-infected T cells. Therapeutic payload-mediated killing of CD19-positive NALM-6 cells by the CD3/CD19 BiTE was only observed with cCAR T cells in the presence of BCMA-positive OPM2 cells (FIG. 8C).

### Example 7: Efficacy of cCAR Cells Against a Multiple Myeloma In Vivo Model

In this example, the present disclosure will be applied in an embodiment to investigate specificity and efficacy of the cCAR system for targeting of heterogenous tumor cell clusters in vivo. Multiple myeloma will again be used as a model system and will initially focus on using the anti-CD19 and CD20 BiTEs as an example of therapeutic payloads detailed in elsewhere herein. Myeloma primagraft models have been challenging to generate to date, however, intramedullary xenograft NOD-scid-IL2Rg^{null} (NSG) models have been successfully employed to mimic the bone marrow stroma (μ-SCID xenograft model), as described in, for example, Bianchi et al., Blood Cancer Discov. 2 (4): 338-353 (2021). To this end, the bilateral femura of NSG donor mice will be harvested, aspirated, and the endogenous bone marrow will be discarded. Then Molp2 or Karpas620 myeloma cells, respectively, will be injected intramedullary prior to sealing the femural head with Matrigel. The femura will be implanted subcutaneously into NSG recipient mice (2 implants per mouse, 7 mice per group). Mice will then be injected with cCAR cells expressing anti-CD38/anti-BCMA/CD3-CD19 BiTE therapeutic payload or cCAR cells expressing anti-CD38/anti-BCMA/CD3-CD20 BiTE therapeutic payload, respectively. To assess the efficacy of the therapeutic payload, Molp2^{CD38 KO/BCMA KO} or Karpas620^{CD38 KO/BCMA KO} cells will be co-implanted, respectively, with and without co-implantation of wild-type Molp2 or Karpas620 cells. Due to the cytotoxicity of the BiTE therapeutic payload, both wildtype and knock-out myeloma cells are expected to be effectively killed, but only if the wild-type Molp2 or Karpas620 cells are present. Tumor killing will be assessed for tumor burden using luminescence and generating Kaplan-Meier survival curves.

In a second set of experiments, non-cancerous B-cells expressing CD19 or CD20 death from BiTE-mediated killing will be assessed. To this end, NSG recipient mice will be engrafted with normal donor B-cells. Once engraftment has been confirmed by peripheral blood flow cytometry, femoral grafts containing Molp2 or Karpas620 myeloma cells will be implant and co-transfected with the respective CD38/BCMA KO myeloma cells as detailed above. Then cCAR cells expressing anti-CD38/anti-BCMA/CD3-CD19 BiTE therapeutic payload or cCAR cells expressing anti-CD38/anti-BCMA/CD3-CD20 BiTE therapeutic payload will be injected, respectively, and disease burden will be monitored as described above. In addition, the number of non-cancerous B-cells in peripheral blood, non-cancerous bone marrow, and bone marrow myeloma graft will be assessed by flow cytometric staining for CD19, CD20 and CD79a. A decrease in B-cells in the myeloma bone marrow graft but no effect on the number of B-cells in peripheral blood and non-cancerous bone marrow is expected.

These experiments will establish the efficacy of the cCAR-dependent therapeutic payload for i) targeting of heterogenous tumor cell clusters and ii) sparing of normal tissue localized in separate compartments.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A cluster Chimeric Antigen Receptor (cCAR) system, comprising:
at least one of a first nucleic acid, a second nucleic acid, and a third nucleic acid, wherein the first nucleic acid comprises a first promotor operably linked to a nucleic acid encoding a first chimeric antigen receptor (CAR) comprising a first extracellular domain comprising a first antigen binding domain that binds a first tumor associated antigen (TAA), a first transmembrane domain, and a first intracellular domain comprising a first signaling domain, and a protease domain;
the second nucleic acid comprises a second promotor operably linked to a nucleic acid encoding a second CAR comprising a second extracellular domain comprising a second antigen binding domain that binds a second TAA, a second transmembrane domain, and an intracellular domain comprising a second signaling domain, a cleavage site recognized by the protease, and a transcriptional activator; and
the third nucleic acid comprises a transcriptional acceptor that binds the transcriptional activator, a third promoter and a nucleic acid encoding a leader peptide and a therapeutic payload that is operatively linked to the third promoter.

2. The cCAR system of claim 1, wherein the first, the second, and the third nucleic acids are disposed in one vector, optionally a lentiviral vector.

3. The cCAR system of claim 1, wherein two of the first, the second, and the third nucleic acids are disposed in a first vector, and the third of the three nucleic acids is disposed in a second vector.

4. The cCAR system of claim 1, wherein the first promoter, the second promoter, or both the first and the second promoters are an EF-1α, CMV, PGK, RPBSA, AmpR, or CAG promoter.

5. The cCAR system of claim 4, wherein the first and the second promoters are an EF-1α promoter.

6. The cCAR system of claim 1, wherein the first antigen binding domain, the second antigen binding domain, or both the first and the second antigen binding domains bind B-cell maturation antigen (BCMA), CD19, CD20, CD38, CD138, FCRH5, GPRC5D, or SLAMF7.

7. The cCAR system of claim 6, wherein the first and the second antigen binding domains bind BCMA.

8. The cCAR system of claim 7, wherein the first or the second antigen binding domain comprises a VL domain comprising the amino acid sequence
(SEQ ID NO: 10)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWT
FGQGTKLEIK
and a VH domain comprising the amino acid sequence
(SEQ ID NO: 11)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS.

9-16. (canceled)

17. The cCAR system of claim 1, wherein the first or the second transmembrane domain is derived from CD3, CD8α, CD28, or CD137.

18-19. (canceled)

20. The cCAR system of claim 1, wherein the first extracellular domain further comprises a first hinge domain disposed between the first antigen binding domain and the first transmembrane domain, and wherein the second extracellular domain further comprises a second hinge domain disposed between the second antigen binding domain and the second transmembrane domain.

21-29. (canceled)

30. The cCAR system of claim 1, wherein the protease domain is derived from Tobacco Etch Virus protease (TEVp) and the cleavage site comprises a sequence cleavable by TEVp.

31. The cCAR system of claim 30, wherein the cleavage site comprises the amino acid sequence ENLYFQM (SEQ ID NO: 83).

32. The cCAR system of claim 1, wherein the transcriptional activator comprises a Gal4-VP64 fusion protein, and the transcriptional acceptor comprises a Gal4 binding site and wherein the third promoter is a modified CMV promoter.

33. The cCAR system of claim 1, wherein the therapeutic payload comprises an antibody fragment, a cytokine, a soluble cytokine receptor, a chemokine, a soluble chemokine receptor, an RNA or oligopeptide vaccine, or a surface receptor.

34-59. (canceled)

60. A genetically modified immune cell, comprising the cCAR system of claim 1, optionally wherein the immune cell is a T cell (e.g., a CD8+ T cell) or an NK cell.

61-73. (canceled)

74. The cCAR system of claim 1, wherein the first signaling domain, the second signaling domain, or both the first and the second signaling domains comprise a primary signaling domain, a co-stimulatory signaling domain, or both a primary signaling domain and a co-stimulatory signaling domain.

75. The cCAR system of claim 1, wherein the first CAR further comprises a first linker that are N-terminal to the protease domain, and wherein the second CAR further comprises a second linker that are N-terminal to the cleavage site.

76. A method of producing a genetically modified immune cell, comprising introducing the cCAR system of claim 1 into an immune cell.

77. A pharmaceutical composition comprising a therapeutically effective number of the immune cells of claim 60, and a pharmaceutically acceptable carrier.

78. A method of treating cancer, comprising:
administering, to a subject in need thereof, the pharmaceutical composition of claim 77.


SEQUENCE LISTING (127 sequences)

SEQ ID NO: 1 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 4 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 504 aa, synthetic construct
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 6 - protein, 586 aa, synthetic construct
VSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 400 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLT

SEQ ID NO: 8 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 10 - protein, 107 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 11 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 12 - protein, 449 aa, synthetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 211 aa, synthetic construct
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, synthetic construct
XVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 213 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 451 aa, synthetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 218 aa, synthetic construct
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 219 aa, synthetic construct; identical to SEQ ID NO: 15

SEQ ID NO: 22 - protein, 222 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 23 - protein, 211 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 24 - protein, 451 aa, synthetic construct
QVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 213 aa, synthetic construct
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 447 aa, synthetic construct
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 210 aa, synthetic construct
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 28 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 453 aa, synthetic construct
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 216 aa, synthetic construct
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 36 - protein, 107 aa, synthetic construct [sequence omitted]

SEQ ID NO: 37 - protein, 122 aa, synthetic construct [sequence omitted]

SEQ ID NO: 38 - protein, 107 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIK

SEQ ID NO: 39 - protein, 120 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSS

SEQ ID NO: 40 - protein, 107 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 41 - protein, 119 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 42 - protein, 251 aa, synthetic construct
DIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSSSLKSRLTISRDTSKSQVVLTLTNVDPVDTATYYCARMRLPYGMDVWGQGTTVTVSS

SEQ ID NO: 43 - protein, 449 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 214 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 107 aa, synthetic construct; identical to SEQ ID NO: 40

SEQ ID NO: 46 - protein, 119 aa, synthetic construct; identical to SEQ ID NO: 41

SEQ ID NO: 47 - protein, 21 aa, Homo sapiens
LCYLLDGILFIYGVILTALFL

SEQ ID NO: 48 - protein, 22 aa, Homo sapiens
MALIVLGGVAGLLLFIGLGIFF

SEQ ID NO: 49 - protein, 24 aa, Homo sapiens
IYIWAPLAGTCGVLLLSLVITLYC

SEQ ID NO: 50 - protein, 27 aa, Homo sapiens
FWVLVVVGGVLACYSLLVTVAFIIFWV

SEQ ID NO: 51 - protein, 27 aa, Homo sapiens
IISFFLALTSTALLFLLFFLTLRFSVV

SEQ ID NO: 52 - protein, 9 aa, Homo sapiens
QSFGLLDPK

SEQ ID NO: 53 - protein, 23 aa, Homo sapiens
LSEGDKVKMDSRIQVLSRGVNQT

SEQ ID NO: 54 - protein, 49 aa, Homo sapiens
KPTTTPAPRPPTPAPTIASQPLSKRPEACRPAAGGAVHTRGLDFACDIY

SEQ ID NO: 55 - protein, 39 aa, Homo sapiens
IEVMYPPPYLDNERSNGTIIHVKGKHLCPSPLFPGPSKP

SEQ ID NO: 56 - protein, 22 aa, Homo sapiens
EPKSCDKTHTCPPCPAPELLGG

SEQ ID NO: 57 - protein, 18 aa, Homo sapiens
ERKCCVECPPCPAPPAAA

SEQ ID NO: 58 - protein, 19 aa, Homo sapiens
ESKYGPPCPPCPAPEFLGG

SEQ ID NO: 59 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 55 aa, Homo sapiens
KNRKAKAKPVTRGAGAGGRQRGQNKERPPPVPNPDYEPIRKGQRDLYSGLNQRRI

SEQ ID NO: 62 - protein, 41 aa, Homo sapiens
RSKRSRLLHSDYMNMTPRRPGPTRKHYQPYAPPRDFAAYRS

SEQ ID NO: 63 - protein, 48 aa, Homo sapiens
QRRKYRSNKGESPVEPAEPCHYSCPREEEGSTIPIQEDYRKPEPACSP

SEQ ID NO: 64 - protein, 41 aa, Homo sapiens; identical to SEQ ID NO: 62

SEQ ID NO: 65 - protein, 22 aa, Homo sapiens
MIETYNQTSPRSAATGLPISMK

SEQ ID NO: 66 - protein, 25 aa, Homo sapiens
TYCFAPRCRERRRNERLRRESVRPV

SEQ ID NO: 67 - protein, 61 aa, Homo sapiens
KWKKKKRPRNSYKCGTNTMEREESEQTKKREKIHIPERSDEAQRVFKSSKTSSCDKSDTCF

SEQ ID NO: 68 - protein, 42 aa, Homo sapiens
KRGRKKLLYIFKQPFMRPVQTTQEEDGCSCRFPEEEEGGCEL

SEQ ID NO: 69 - protein, 38 aa, Homo sapiens
CWLTKKKYSSSVHDPNGEYMFMRAVNTAKKSRLTDVTL

SEQ ID NO: 70 - protein, 52 aa, Homo sapiens
YFLGRLVPRGRGAAEAATRKQRITETESPYQELQGQRSDVYSDLNTQRPYYK

SEQ ID NO: 71 - protein, 42 aa, Homo sapiens
RLKIQVRKAAITSYEKSDGVYTGLSTRNQETYETLKHEKPPQ

SEQ ID NO: 72 - protein, 41 aa, Homo sapiens; identical to SEQ ID NO: 62

SEQ ID NO: 73 - protein, 39 aa, Homo sapiens
HRWCSNKKNAAVMDQEPAGNRTVNSEDSDEQDHQEVSYA

SEQ ID NO: 74 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 75 - protein, 42 aa, Homo sapiens
ALYLLRRDQRLPPDAHKPPGGGSFRTPIQEEQADAHSTLAKI

SEQ ID NO: 76 - protein, 619 aa, Homo sapiens
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 77 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 78 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 79 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 80 - protein, 234 aa, Homo sapiens
MPCGLSALIMCPKGMVAVVVAVDDGDSQALAGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 81 - protein, 219 aa, Tobacco etch virus
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMV

SEQ ID NO: 82 - protein, 7 aa, Tobacco etch virus
ENLYFQX

SEQ ID NO: 83 - protein, 7 aa, Tobacco etch virus
ENLYFQM

SEQ ID NO: 84 - DNA, 9898 bp, synthetic construct
  source 1..9898: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattcgaattcgccgccaccatggccttaccagtgaccgccttgctcctgccgctggccttgctgctccacgccgccaggccggagcagaagctcatctccgaggaggacctggatatccagatgacccagagcccgtcgagcctttccgcctccgtgggggacagggtcactatcacctgctctgcttcccaggacatctctaactacctgaattggtaccagcagaagcccggcaaggcgcccaagctactcatttactacaccagcaacctgcactctggtgtgcctagccgcttttcaggttccggctccggcaccgacttcaccctgactatttcgagtttgcagccagaggacttcgccacctactattgtcaacagtaccgcaagctgccgtggaccttcggacagggcacaaaactggagatcaagggtggcggtggctcgggcggtggtgggtcgggtggcggcggatctcaagtgcagctggtgcagagcggggcagaagtcaagaagcctggctcctctgtaaaggtgtcatgcaaggcttccggtggcacgttcagcaactattggatgcattgggtccgccaggcccccggacagggcctggagtggatgggggccacctaccgtggccacagcgatacttactacaaccagaaatttaaaggccgcgtgaccatcaccgcggacaagtcgacctccacagcctacatggagctgtctagtttgcgctcggaggacactgctgtttattactgtgcgcggggtgccatttacgacggctacgatgtgctcgacaattggggccagggaacccttgtcaccgtgtcctctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtggcggctccggggagagcctgttcaagggccctcgtgactacaatccaatttcgtcgaccatctgtcacttgacaaacgagtccgatggtcataccacttctctgtacggcatcggtttcggacccttcattataaccaacaagcacttgttccgccgcaacaacggcaccctgcttgtgcagagcctacatggagtttttaaggtcaaaaacacgaccactctgcaacagcacctgattgacggccgggacatgatcatcatccgtatgcccaaggacttccccccgtttcctcagaagctcaaatttcgcgagcctcagagggaggagcgcatctgtctggtcaccacaaatttccagaccaagtctatgtcttccatggtgagtgacacctcatgcactttcccgtcttccgatggtatcttctggaagcactggatccagacgaaagatggacagtgcgggtcccccctggtgtccactcgcgacggcttcatcgtgggcatccactcggcctcaaatttcaccaacacgaacaactatttcacctccgtgccaaagaactttatggaactgctgaccaaccaggaggctcagcagtgggtcagcggctggcgactcaacgcggacagcgtactttggggcgggcacaaggtgttcatggtgtaataacatatgcctaggtctagaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 85 - protein, 10 aa, synthetic construct
EQKLISEEDL

SEQ ID NO: 86 - protein, 211 aa, synthetic construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 87 - protein, 5 aa, synthetic construct
GGGGX

SEQ ID NO: 88 - protein, 6 aa, synthetic construct
GSSGSX

SEQ ID NO: 89 - protein, 4 aa, synthetic construct
GGGC

SEQ ID NO: 90 - protein, 4 aa, synthetic construct
GGGS

SEQ ID NO: 91 - protein, 15 aa, synthetic construct
GGGGSGGGGSGGGGS

SEQ ID NO: 92 - protein, 20 aa, synthetic construct
GGGGSGGGGSGGGGSGGGGS

SEQ ID NO: 93 - protein, 18 aa, synthetic construct
GSTSGSGKPGSGEGSTKG

SEQ ID NO: 94 - protein, 18 aa, synthetic construct
KESGSVSSEQLAQFRSLD

SEQ ID NO: 95 - protein, 14 aa, synthetic construct
EGKSSGSGSESKST

SEQ ID NO: 96 - protein, 12 aa, synthetic construct
GSAGSAAGSGEF

SEQ ID NO: 97 - DNA, 10984 bp, synthetic construct
  source 1..10984: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattcgaattcgccgccaccatggccttaccagtgaccgccttgctcctgccgctggccttgctgctccacgccgccaggccggactacaaggacgacgatgacaaggatatccagatgacccagagcccgtcgagcctttccgcctccgtgggggacagggtcactatcacctgctctgcttcccaggacatctctaactacctgaattggtaccagcagaagcccggcaaggcgcccaagctactcatttactacaccagcaacctgcactctggtgtgcctagccgcttttcaggttccggctccggcaccgacttcaccctgactatttcgagtttgcagccagaggacttcgccacctactattgtcaacagtaccgcaagctgccgtggaccttcggacagggcacaaaactggagatcaagggtggcggtggctcgggcggtggtgggtcgggtggcggcggatctcaagtgcagctggtgcagagcggggcagaagtcaagaagcctggctcctctgtaaaggtgtcatgcaaggcttccggtggcacgttcagcaactattggatgcattgggtccgccaggcccccggacagggcctggagtggatgggggccacctaccgtggccacagcgatacttactacaaccagaaatttaaaggccgcgtgaccatcaccgcggacaagtcgacctccacagcctacatggagctgtctagtttgcgctcggaggacactgctgtttattactgtgcgcggggtgccatttacgacggctacgatgtgctcgacaattggggccagggaacccttgtcaccgtgtcctctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagaacctgtacttccagatgatgaaactgctgagctcgattgagcaggcctgcgacatctgtagactcaagaagctgaagtgctctaaggagaaaccaaaatgtgccaagtgcctgaagaacaactgggaatgtcgctactcccccaagacaaagcgcagccctctgacccgcgcccacttgaccgaggtggagagccgtctggagcgcctggaacagctgttcctgctcatcttcccgagggaggacctggacatgatcctgaaaatggattcgctgcaggacatcaaggctcttctgaccggcctcttcgtgcaggacaacgtgaacaaggacgccgtcaccgaccgcctggcctctgtggagactgacatgcccctgacgctacggcaacatcgtatttctgccacctcctcgtccgaggagagctcaaataagggccagcgccagcttactgttagtgctgcggctggcgggtccggtgggtctggcggttccgacgctctggatgactttgatctggacatgttgggaagtgatgcgctcgatgatttcgacttggacatgcttggctccgacgcacttgatgacttcgacctcgacatgttgggaagcgacgcgctggacgattttgacctggacatgctaggctccggcgcgccggagggcagaggcagcctgctgacctgcggcgacgtggaggagaaccccggccccatgtggctgcagagcctgctgctcttgggcactgtggcctgcagcatctctcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtaataatctagaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 98 - protein, 8 aa, synthetic construct
DYKDDDDK

SEQ ID NO: 99 - DNA, 20 bp, synthetic construct
  source 1..20: mol_type = other DNA
ggagcactgtcctccgaacg

SEQ ID NO: 100 - protein, 18 aa, Homo sapiens
MKWVTFISLLFLFSSAYS

SEQ ID NO: 101 - protein, 19 aa, synthetic construct
MKWVTFISLLFLFSSSSRA

SEQ ID NO: 102 - protein, 21 aa, Homo sapiens
MALPVTALLLPLALLLHAARP

SEQ ID NO: 103 - protein, 16 aa, Homo sapiens
MPLLLLLPLLWAGALA

SEQ ID NO: 104 - protein, 27 aa, Homo sapiens
MGVHECPAWLWLLLSLLSLPLGLPVLG

SEQ ID NO: 105 - protein, 20 aa, Homo sapiens
MYRMQLLSCIALSLALVTNS

SEQ ID NO: 106 - protein, 20 aa, Homo sapiens
METDTLLLWVLLLWVPGSTG

SEQ ID NO: 107 - protein, 20 aa, Homo sapiens
MEAPAQLLFLLLLWLPDTTG

SEQ ID NO: 108 - protein, 20 aa, Homo sapiens
MVLQTQVFISLLLWISGAYG

SEQ ID NO: 109 - protein, 20 aa, synthetic construct
MEAPAQLLFLLLLWLPSSRA

SEQ ID NO: 110 - protein, 23 aa, Homo sapiens
MDAMKRGLCCVLLLCGAVFVSPS

SEQ ID NO: 111 - protein, 17 aa, Homo sapiens
MLLLLLLLGLRLQLSLG

SEQ ID NO: 112 - protein, 16 aa, synthetic construct
MLLLLLLLLLLALALA

SEQ ID NO: 113 - protein, 21 aa, synthetic construct
MWWRLWWLLLLLLLLWPMVWA

SEQ ID NO: 114 - protein, 452 aa, synthetic construct
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 214 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 117 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 118 - protein, 498 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 119 - protein, 493 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIKGGGGSGGGGSGGGGSEVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSSGGGGSEVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSGGGGSGGGGSGGGGSDIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 120 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 121 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 122 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 123 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 124 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 125 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 126 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 127 - DNA, 8508 bp, synthetic construct
  source 1..8508: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgatatcgaattcggagcactgtcctccgaacgtcggagcactgtcctccgaacgtcggagcactgtcctccgaacgtcggagcactgtcctccgaacggagcatgtcctccgaacgtcggagcactgtcctccgaacgactagttaggcgtgtacggtgggaggcctatataagcagagctcgtttagtgaaccgtcagatcgcctggagacgccatccacgctgttttgacctccatagaagacaccgactctagaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctcgagcagcgctgcgatcgcgttaacgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacatatgcctaggtctagaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt